(19) United States
(12) Patent Application Publication
(10) Pub. No.: US 2025/0346668 A1
(43) Pub. Date: Nov. 13, 2025

(54) CD3-TARGETING ANTIBODY AND USE THEREOF

(71) Applicant: GENOR BIOPHARMA CO., LTD, Shanghai (CN)

(72) Inventors: Qian Ding, Shanghai (CN); Xuekun Zhang, Shanghai (CN); Fei Peng, Shanghai (CN); Jian Li, Shanghai (CN); Meng Feng, Shanghai (CN); Linlin Zhang, Shanghai (CN); Zhen Li, Shanghai (CN)

(21) Appl. No.: 18/850,279

(22) PCT Filed: Mar. 25, 2022
(86) PCT No.: PCT/CN2022/083004

§ 371 Date: Sep. 24, 2024

(51) Int. Cl. C07K 16/28

(52) U.S. Cl. CPC C07K 16/2809; C07K 2317/24

(57) ABSTRACT

The present disclosure relates to a CD3-targeting antibody and use thereof. Specifically, the present disclosure provides an antibody or antigen-binding fragment thereof that specifically binds to CD3, a nucleic acid encoding the antibody or antigen-binding fragment thereof, a nucleic acid vector comprising the nucleic acid, a host cell comprising the nucleic acid or nucleic acid vector, a pharmaceutical composition comprising the aforementioned substances, and a preparation method therefor and use thereof.

### CROSS-REFERENCE TO RELATED APPLICATION

This patent application is the U.S. national stage entry, under 35 U.S.C. § 371, of International Application Number PCT/CN2022/083004, filed Mar. 25, 2022, the entire contents of which is hereby incorporated by reference.

### REFERENCE TO A SEQUENCE LISTING

This application is filed with a Computer Readable Form of a Sequence Listing in accord with 37 C.F.R. § 1.821 (c). The text file submitted in the USPTO Patent Center, “023704-0007-US01_sequence_listing_30 Apr. 2025_ST25.txt,” was created on Apr. 30, 2025, contains 92 sequences, has a file size of 80.0 kilobytes (81,920 bytes), and is hereby incorporated by reference in its entirety.

### TECHNICAL FIELD

The present application relates to the field of biomedicine, and in particular to a CD3-targeting antibody or an antigen-binding fragment thereof, and a preparation method and use thereof.

### BACKGROUND ART

The TCR/CD3 complex is the main regulator of T cell function and specificity in humans and other mammals, and plays a very important role in antigen recognition and intracellular signal transduction pathways. Among it, CD3 is a protein complex composed of four different chains (CD38 chain, CD3γ chain, CD3ζ chain and CD38 chain), which contains three dimers, namely CD3ε/γ (or CD3e/g), CD3ε/δ (or CD3e/d) and CD3ζ/ζ (or CD3z/z), and their intracellular regions all contain immunoreceptor tyrosine-based activation motifs (ITAMs) necessary for initiating signal transduction. TCR is a heterodimer of covalently linked α and β chains (“TCRαβ”), which mainly recognizes antigens presented by MHC molecules, but its intracellular region is very short and needs to form a TCR/CD3 complex with CD3 to transduce stimulation signals into a cell (Dong, D., Zheng, L., Lin, J. et al. Nature 573, 546-552 (2019)).

At present, antibody drugs targeting CD3 have been widely used in clinical practice. In 1986, the first monoclonal antibody drug approved by the FDA was Muromonab (also known as OKT3), a monoclonal antibody to CD3, which was used to treat acute rejection after organ transplantation. After that, other CD3 targeting monoclonal antibodies, such as Otelixizumab, Teplizumab, and Visilizumab, were also developed for the treatment of Crohn's disease, ulcerative colitis, and type I diabetes. In recent years, due to the important role of CD3 in the process such as activation and proliferation of T cells, bispecific or multispecific antibodies targeting human CD3 and tumor-associated antigens (TAAs) have also become a research hotspot in anti-tumor therapy.

The difficulty in developing a CD3 targeting antibody (used interchangeably with “CD3 antibody”, “anti-CD3 antibody”, and “antibody that specifically binds to CD3” in this disclosure) is that it is difficult to obtain a good immune response and serum titer, resulting in a small number of positive clones after fusion. Currently commercially available CD3 antibodies include OKT3. After the end of treatment with OKT3, T cell function usually returns to normal within a week, but the side effects of OKT3 at the beginning of use are also considerable, including OKT3 flu-like syndrome, cytokine storm, etc. In addition, OKT3 does not bind to CD3 derived from cynomolgus, which brings inconvenience to the selection of primate cynomolgus in preclinical safety evaluation studies.

The present disclosure provides a new anti-CD3 antibody, which can bind to human and cynomolgus CD3, has low side effects, and provides a new potential treatment for diseases including cancer and autoimmune diseases. The antibody or antigen-binding fragment thereof in the present disclosure has excellent biological activities (e.g., high affinity). It can bind to human CD3e/g (hCD3e/g), human CD3e/d (hCD3e/d) and cynomolgus CD3e/g (cCD3e/g), so that the disease model of primate cynomolgus (cyno) can be selected for pharmacological and toxicological experiments in preclinical safety assessment studies, which greatly facilitates preclinical pharmacology, toxicology and other studies. In addition, the antibody or antigen-binding fragment thereof in the present disclosure can effectively activate the NFAT downstream signaling pathway of human T cell line Jurkat cells, which is conducive to the subsequent biological activity.

### SUMMARY OF THE INVENTION

The present disclosure relates to an antibody or antigen-binding fragment thereof that specifically binds to CD3 and uses thereof.

In a first aspect, the present disclosure relates to an antibody or antigen-binding fragment thereof that specifically binds to CD3, the heavy chain of which comprises CDR1, CDR2 and CDR3, wherein:
(a) the heavy chain CDR1 comprises a sequence that is at least 80% identical to NYYMH (SEQ ID NO: 1), DNYIH (SEQ ID NO: 2), DYTVN (SEQ ID NO: 3) or DYTIH (SEQ ID NO: 4);
(b) the heavy chain CDR2 comprises a sequence that is at least 80% identical to WTYPGNNNIKYNEKFKG (SEQ ID NO: 18), WIYPGSVNIKYNEKFKD (SEQ ID NO: 6), or YINPFNSYTKYNQKFKD (SEQ ID NO: 22); and
(c) the heavy chain CDR3 comprises a sequence that is at least 80% identical to DGYGYYFFDY (SEQ ID NO: 8), DISRYYFDY (SEQ ID NO: 9), SVSTY (SEQ ID NO: 10) or SVSIY (SEQ ID NO: 11).

In some embodiments, the antibody or antigen-binding fragment thereof comprises:
(a) a heavy chain CDR1 comprising a sequence selected from the group consisting of NYYMH (SEQ ID NO: 1), DNYIH (SEQ ID NO: 2), DYTVN (SEQ ID NO: 3), DYTIH (SEQ ID NO: 4), or a sequence having one amino acid addition, substitution and/or deletion compared to any of SEQ ID NOs: 1-4;
(b) a heavy chain CDR2 comprising a sequence selected from the group consisting of: WTYPGX₁X₂X₃IKYNEKFKG (SEQ ID NO: 5), WIYPGSVNIKYNEKFKD (SEQ ID NO: 6) or a sequence that differs from SEQ ID NO: 6 by no more than a total of 3 amino acid additions, substitutions and/or deletions, or YINPFX₄X₅YTKYNQKFKD (SEQ ID NO: 7); wherein X₁, X₂ and X₃ are each independently N or Q, X₄ is N or S, X₅ is S or D;
(c) a heavy chain CDR3 comprising a sequence selected from the group consisting of: DGYGYYFFDY (SEQ ID NO: 8), DISRYYFDY (SEQ ID NO: 9), SVSTY (SEQ ID NO: 10), SVSIY (SEQ ID NO: 11), or a sequence that differs from any of SEQ ID NOs: 8-11 by no more than a total of 2 amino acid additions, substitutions and/or deletions;
(d) a light chain CDR1 comprising a sequence selected from the group consisting of: KSSQSLLNX₆RTRKNYLA (SEQ ID NO: 12) or KSSQSLLDX₇DX₈KTYLN (SEQ ID NO: 13); wherein X₆ is N, Q or S, X₇ is S, A or G, X₈ is G or A;
(e) a light chain CDR2 comprising a sequence selected from the group consisting of: WASTRES (SEQ ID NO: 14) or LVSKLNS (SEQ ID NO: 15); and
(f) a light chain CDR3 comprising a sequence selected from the group consisting of: KQSX₉X₁₀LRT (SEQ ID NO: 16) or WQGTHFPRT (SEQ ID NO: 17); wherein X₉ is Y or F, and X₁₀ is T or I.

In some embodiments, the antibody or antigen-binding fragment thereof comprises:
(1) a heavy chain CDR1 comprising or consisting of the following sequence: NYYMH (SEQ ID NO: 1); a heavy chain CDR2 comprising or consisting of the following sequence: WTYPGX₁X₂X₃IKYNEKFKG (SEQ ID NO: 5), wherein X₁, X₂ and X₃ are each independently N or Q; a heavy chain CDR3 comprising or consisting of the following sequence: DGYGYYFFDY (SEQ ID NO: 8); a light chain CDR1 comprising or consisting of the following sequence: KSSQSLLNX₆RTRKNYLA (SEQ ID NO: 12), wherein X₆ is N or Q; a light chain CDR2 comprising or consisting of the following sequence: WASTRES (SEQ ID NO: 14); and a light chain CDR3 comprising or consisting of the following sequence: KQSYTLRT (SEQ ID NO: 31); or
(2) a heavy chain CDR1 comprising or consisting of the following sequence: DNYIH (SEQ ID NO: 2); a heavy chain CDR2 comprising or consisting of the following sequence: WIYPGSVNIKYNEKFKD (SEQ ID NO: 6); a heavy chain CDR3 comprising or consisting of the following sequence: DISRYYFDY (SEQ ID NO: 9); a light chain CDR1 comprising or consisting of the following sequence: KSSQSLLNSRTRKNYLA (SEQ ID NO: 26); a light chain CDR2 comprising or consisting of the following sequence: WASTRES (SEQ ID NO: 14); and a light chain CDR3 comprising or consisting of the following sequence: KQSFILRT (SEQ ID NO: 32); or
(3) a heavy chain CDR1 comprising or consisting of the following sequence: DYTVN (SEQ ID NO: 3); a heavy chain CDR2 comprising or consisting of the following sequence: YINPFNSYTKYNQKFKD (SEQ ID NO: 22); a heavy chain CDR3 comprising or consisting of the following sequence: SVSTY (SEQ ID NO: 10); a light chain CDR1 comprising or consisting of the following sequence: KSSQSLLDX₇DX₈KTYLN (SEQ ID NO: 13), wherein X₇ is S, and X₈ is G or A; a light chain CDR2 comprising or consisting of the following sequence: LVSKLNS (SEQ ID NO: 15); and a light chain CDR3 comprising or consisting of the following sequence: WQGTHFPRT (SEQ ID NO: 17); or
(4) a heavy chain CDR1 comprising or consisting of the following sequence: DYTIH (SEQ ID NO: 4); a heavy chain CDR2 comprising or consisting of the following sequence: YINPFSDYTKYNQKFKD (SEQ ID NO: 23); a heavy chain CDR3 comprising or consisting of the following sequence: SVSIY (SEQ ID NO: 11); a light chain CDR1 comprising or consisting of the following sequence: KSSQSLLDX₇DX₈KTYLN (SEQ ID NO: 13), wherein X₇ is G or A, and X₈ is G; a light chain CDR2 comprising or consisting of the following sequence: LVSKLNS (SEQ ID NO: 15); and a light chain CDR3 comprising or consisting of the following sequence: WQGTHFPRT (SEQ ID NO: 17).

In some embodiments, the heavy chain CDR2 comprises a sequence selected from the group consisting of WTYPGNNNIKYNEKFKG (SEQ ID NO: 18), WTYPGQNNIKYNEKFKG (SEQ ID NO: 19), WTYPGNQNIKYNEKFKG (SEQ ID NO: 20), WTYPGQNQIKYNEKFKG (SEQ ID NO: 21), WIYPGSVNIKYNEKFKD (SEQ ID NO: 6), YINPFNSYTKYNQKFKD (SEQ ID NO: 22) and YINPFSDYTKYNQKFKD (SEQ ID NO: 23). In some embodiments, the light chain CDR1 comprises a sequence selected from the group consisting of KSSQSLLNNRTRKNYLA (SEQ ID NO: 24), KSSQSLLNQRTRKNYLA (SEQ ID NO: 25), KSSQSLLNSRTRKNYLA (SEQ ID NO: 26), KSSQSLLDSDGKTYLN (SEQ ID NO: 27), KSSQSLLDSDAKTYLN (SEQ ID NO: 28), KSSQSLLDGDGKTYLN (SEQ ID NO: 29) and KSSQSLLDADGKTYLN (SEQ ID NO: 30). In some embodiments, the light chain CDR3 comprises a sequence selected from the group consisting of KQSYTLRT (SEQ ID NO: 31), KQSFILRT (SEQ ID NO: 32) or WQGTHFPRT (SEQ ID NO: 17).

In some embodiments, the antibody or antigen-binding fragment thereof comprises:
(1) a heavy chain CDR1 comprising or consisting of the following sequence: NYYMH (SEQ ID NO: 1); a heavy chain CDR2 comprising or consisting of the following sequence: WTYPGNNNIKYNEKFKG (SEQ ID NO: 18); a heavy chain CDR3 comprising or consisting of the following sequence: DGYGYYFFDY (SEQ ID NO: 8); a light chain CDR1 comprising or consisting of the following sequence: KSSQSLLNNRTRKNYLA (SEQ ID NO: 24); a light chain CDR2 comprising or consisting of the following sequence: WASTRES (SEQ ID NO: 14); and a light chain CDR3 comprising or consisting of the following sequence: KQSYTLRT (SEQ ID NO: 31); or
(2) a heavy chain CDR1 comprising or consisting of the following sequence: NYYMH (SEQ ID NO: 1); a heavy chain CDR2 comprising or consisting of the following sequence: WTYPGNNNIKYNEKFKG (SEQ ID NO: 18); a heavy chain CDR3 comprising or consisting of the following sequence: DGYGYYFFDY (SEQ ID NO: 8); a light chain CDR1 comprising or consisting of the following sequence: KSSQSLLNQRTRKNYLA (SEQ ID NO: 25); a light chain CDR2 comprising or consisting of the following sequence: WASTRES (SEQ ID NO: 14); and a light chain CDR3 comprising or consisting of the following sequence: KQSYTLRT (SEQ ID NO: 31); or
(3) a heavy chain CDR1 comprising or consisting of the following sequence: NYYMH (SEQ ID NO: 1); a heavy chain CDR2 comprising or consisting of the following sequence: WTYPGQNNIKYNEKFKG (SEQ ID NO: 19); a heavy chain CDR3 comprising or consisting of the following sequence: DGYGYYFFDY (SEQ ID NO: 8); a light chain CDR1 comprising or consisting of the following sequence: KSSQSLLNNRTRKNYLA (SEQ ID NO: 24); a light chain CDR2 comprising or consisting of the following sequence: WASTRES (SEQ ID NO: 14); and a light chain CDR3 comprising or consisting of the following sequence: KQSYTLRT (SEQ ID NO: 31); or
(4) a heavy chain CDR1 comprising or consisting of the following sequence: NYYMH (SEQ ID NO: 1); a heavy chain CDR2 comprising or consisting of the following sequence: WTYPGQNNIKYNEKFKG (SEQ ID NO: 19); a heavy chain CDR3 comprising or consisting of the following sequence: DGYGYYFFDY (SEQ ID NO: 8); a light chain CDR1 comprising or consisting of the following sequence: KSSQSLLNQRTRKNYLA (SEQ ID NO: 25); a light chain CDR2 comprising or consisting of the following sequence: WASTRES (SEQ ID NO: 14); and a light chain CDR3 comprising or consisting of the following sequence: KQSYTLRT (SEQ ID NO: 31); or
(5) a heavy chain CDR1 comprising or consisting of the following sequence: NYYMH (SEQ ID NO: 1); a heavy chain CDR2 comprising or consisting of the following sequence: WTYPGNQNIKYNEKFKG (SEQ ID NO: 20); a heavy chain CDR3 comprising or consisting of the following sequence: DGYGYYFFDY (SEQ ID NO: 8); a light chain CDR1 comprising or consisting of the following sequence: KSSQSLLNNRTRKNYLA (SEQ ID NO: 24); a light chain CDR2 comprising or consisting of the following sequence: WASTRES (SEQ ID NO: 14); and a light chain CDR3 comprising or consisting of the following sequence: KQSYTLRT (SEQ ID NO: 31); or
(6) a heavy chain CDR1 comprising or consisting of the following sequence: NYYMH (SEQ ID NO: 1); a heavy chain CDR2 comprising or consisting of the following sequence: WTYPGNQNIKYNEKFKG (SEQ ID NO: 20); a heavy chain CDR3 comprising or consisting of the following sequence: DGYGYYFFDY (SEQ ID NO: 8); a light chain CDR1 comprising or consisting of the following sequence: KSSQSLLNQRTRKNYLA (SEQ ID NO: 25); a light chain CDR2 comprising or consisting of the following sequence: WASTRES (SEQ ID NO: 14); and a light chain CDR3 comprising or consisting of the following sequence: KQSYTLRT (SEQ ID NO: 31); or
(7) a heavy chain CDR1 comprising or consisting of the following sequence: NYYMH (SEQ ID NO: 1); a heavy chain CDR2 comprising or consisting of the following sequence: WTYPGQNQIKYNEKFKG (SEQ ID NO: 21); a heavy chain CDR3 comprising or consisting of the following sequence: DGYGYYFFDY (SEQ ID NO: 8); a light chain CDR1 comprising or consisting of the following sequence: KSSQSLLNNRTRKNYLA (SEQ ID NO: 24); a light chain CDR2 comprising or consisting of the following sequence: WASTRES (SEQ ID NO: 14); and a light chain CDR3 comprising or consisting of the following sequence: KQSYTLRT (SEQ ID NO: 31); or
(8) a heavy chain CDR1 comprising or consisting of the following sequence: NYYMH (SEQ ID NO: 1); a heavy chain CDR2 comprising or consisting of the following sequence: WTYPGQNQIKYNEKFKG (SEQ ID NO: 21); a heavy chain CDR3 comprising or consisting of the following sequence: DGYGYYFFDY (SEQ ID NO: 8); a light chain CDR1 comprising or consisting of the following sequence: KSSQSLLNQRTRKNYLA (SEQ ID NO: 25); a light chain CDR2 comprising or consisting of the following sequence: WASTRES (SEQ ID NO: 14); and a light chain CDR3 comprising or consisting of the following sequence: KQSYTLRT (SEQ ID NO: 31); or
(9) a heavy chain CDR1 comprising or consisting of the following sequence: DYTVN (SEQ ID NO: 3); a heavy chain CDR2 comprising or consisting of the following sequence: YINPFNSYTKYNQKFKD (SEQ ID NO: 22); a heavy chain CDR3 comprising or consisting of the following sequence: SVSTY (SEQ ID NO: 10); a light chain CDR1 comprising or consisting of the following sequence: KSSQSLLDSDGKTYLN (SEQ ID NO: 27); a light chain CDR2 comprising or consisting of the following sequence: LVSKLNS (SEQ ID NO: 15); and a light chain CDR3 comprising or consisting of the following sequence: WQGTHFPRT (SEQ ID NO: 17); or
(10) a heavy chain CDR1 comprising or consisting of the following sequence: DYTVN (SEQ ID NO: 3); a heavy chain CDR2 comprising or consisting of the following sequence: YINPFNSYTKYNQKFKD (SEQ ID NO: 22); a heavy chain CDR3 comprising or consisting of the following sequence: SVSTY (SEQ ID NO: 10); a light chain CDR1 comprising or consisting of the following sequence: KSSQSLLDSDAKTYLN (SEQ ID NO: 28); a light chain CDR2 comprising or consisting of the following sequence: LVSKLNS (SEQ ID NO: 15); and a light chain CDR3 comprising or consisting of the following sequence: WQGTHFPRT (SEQ ID NO: 17); or
(11) a heavy chain CDR1 comprising or consisting of the following sequence: DYTIH (SEQ ID NO: 4); a heavy chain CDR2 comprising or consisting of the following sequence: YINPFSDYTKYNQKFKD (SEQ ID NO: 23); a heavy chain CDR3 comprising or consisting of the following sequence: SVSIY (SEQ ID NO: 11); a light chain CDR1 comprising or consisting of the following sequence: KSSQSLLDADGKTYLN (SEQ ID NO: 30); a light chain CDR2 comprising or consisting of the following sequence: LVSKLNS (SEQ ID NO: 15); and a light chain CDR3 comprising or consisting of the following sequence: WQGTHFPRT (SEQ ID NO: 17); or
(12) a heavy chain CDR1 comprising or consisting of the following sequence: DYTIH (SEQ ID NO: 4); a heavy chain CDR2 comprising or consisting of the following sequence: YINPFSDYTKYNQKFKD (SEQ ID NO: 23); a heavy chain CDR3 comprising or consisting of the following sequence: SVSIY (SEQ ID NO: 11); a light chain CDR1 comprising or consisting of the following sequence: KSSQSLLDGDGKTYLN (SEQ ID NO: 29); a light chain CDR2 comprising or consisting of the following sequence: LVSKLNS (SEQ ID NO: 15); and a light chain CDR3 comprising or consisting of the following sequence: WQGTHFPRT (SEQ ID NO: 17).

In some embodiments, the antibody or antigen-binding fragment thereof comprises a heavy chain variable region comprising the following sequences:

(SEQ ID NO: 33)
QVQLVQSGAEVKKPGASVKVSCKASGYSFTNYYMHWVRQATGQGLEWMG
WTYPGNNNIKYNEKFKGRVTMTRNTSISTAYMELSSLRSEDTAVYYCARDGY
GYYFFDYWGQGTLVTVSS,
(SEQ ID NO: 34)
QVQLVQSGAEVKKPGASVKVSCKASGYSFTNYYMHWVRQATGQGLEWMG
WTYPGQNNIKYNEKFKGRVTMTRNTSSSTAYMELSSLRSEDTAVYYCARDGY
GYYFFDYWGQGTLVTVSS,
(SEQ ID NO: 35)
QVQLVQSGAEVKKPGASVKVSCKASGYSFTNYYMHWVRQATGQGLEWMG
WTYPGNQNIKYNEKFKGKVTMTRNTSISTAYMELSSLRSEDSAVYYCARDGY
GYYFFDYWGQGTIVTVSS,
(SEQ ID NO: 36)
QVQLVQSGAEVKKPGASVKVSCKASGYSFTNYYMHWVRQATGQGLEWMG
WTYPGQNQIKYNEKFKGKVTMTRNTSSSTAYMELSSLRSEDSAVYYCARDGY
GYYFFDYWGQGILVTVSS,
(SEQ ID NO: 37)
QVQLVQSGAEVKKPGASVKVSCKASGYSFTNYYMHWVRQAPGQRLEWMG
WTYPGNNNIKYNEKFKGRVTITRDTSASTAYMELSSLRSEDTAVYYCARDGY
GYYFFDYWGQGTLVTVSS,
(SEQ ID NO: 38)
QVQLVQSGAEVKKPGASVKVSCKASGYSFTNYYMHWVRQAPGQRLEWIGW
TYPGNNNIKYNEKFKGRVTITADTSASTAYMELSSLRSEDTAVYYCARDGYG
YYFFDYWGQGTLVTVSS,
(SEQ ID NO: 39)
QVQLVQSGAEVKKPGASVKVSCKASGYSFTNYYMHWVRQAPGQRLEWIGW
TYPGNNNIKYNEKFKGRATITADTSASTAYMELSSLRSEDTAVYYCARDGYG
YYFFDYWGQGTLVTVSS,
(SEQ ID NO: 40)
QVQLVQSGAEVKKPGASVKVSCKASGYSFTNYYMHWVKQAPGQRLEWIGW
TYPGNNNIKYNEKFKGRATITADTSASTAYMELSSLRSEDTAVYYCARDGYG
YYFFDYWGQGTLVTVSS,
(SEQ ID NO: 41)
QVQLVQSGAEVKKPGASVKVSCKASGYTFTDNYIHWVRQATGQGLEWMGW
IYPGSVNIKYNEKFKDRVTMTRNTSISTAYMELSSLRSEDTAVYYCARDISR
YYFDYWGQGTLVTVSS,
(SEQ ID NO: 42)
QVQLVQSGAEVKKPGASVKVSCKASGSTFTDNYIHWVRQATGQGLEWMGW
IYPGSVNIKYNEKFKDKVTLTRNTSISTAYMELSSLRSEDSAVYYCARDISR
YYFDYWGQGTLVTVSS,
(SEQ ID NO: 43)
QVQLVQSGAEVVKPGASVKVSCKASGYTFTDNYIHWVRQRTGQGLEWMGW
IYPGSVNIKYNEKFKDRVTMTADTSISTAYMQLSSLSSEDTAVYYCARDISR
YYFDYWGQGTLVTVSS,
(SEQ ID NO: 44)
QVQLVQSGAELVKPGASVKVSCKASGSTFTDNYIHWVRQRTGQGLEWMGWI
YPGSVNIKYNEKFKDRVTLTADTSITTAYMQLSSLSSEDTAIYYCARDISRY
YFDYWGQGTLVTVSS,
(SEQ ID NO: 45)
QVQLVQSGAEVKKPGASVKVSCKASGSTFTDNYIHWVRQAPGQRLEWMGW
IYPGSVNIKYNEKFKDRVTITRDTSASTAYMELSSLRSEDTAVYYCARDISR
YYFDYWGQGTTVTVSS,
(SEQ ID NO: 46)
QVQLVQSGAEVKKPGASVKVSCKASGSTFTDNYIHWVRQAPGQRLEWIGWI
YPGSVNIKYNEKFKDRVTITADTSASTAYMELSSLRSEDTAVYYCARDISRY
YFDYWGQGTTVTVSS,
(SEQ ID NO: 47)
QVQLVQSGAEVKKPGASVKVSCKASGSTFTDNYIHWVRQAPGQRLEWIGWI
YPGSVNIKYNEKFKDRATITADTSASTAYMELSSLRSEDTAVYYCARDISRY
YFDYWGQGTTVTVSS,
(SEQ ID NO: 48)
QVQLVQSGAEVKKPGASVKVSCKASGSTFTDNYIHWVKQAPGQRLEWIGWI
YPGSVNIKYNEKFKDRATITADTSATTAYMELSSLRSEDTAVYYCARDISRY
YFDYWGQGTTVTVSS,
(SEQ ID NO: 49)
QVQLVQSGAEVKKPGSSVKVSCKASGYTFTDYTVNWVRQAPGQGLEWMGY
INPFNSYTKYNQKFKDRVTITADESTSTAYMELSSLRSEDTAVYYCANSVST
YWGQGTLVTVSS,
(SEQ ID NO: 50)
QVQLVQSGAEVAKPGSSVKVSCKASGYTFTDYTVNWVRQRPGQGLEWMGYI
NPFNSYTKYNQKFKDRVTITADESTSTAYMELSSLRSEDTAVYYCANSVSTY
WGQGTLVTVSS,
(SEQ ID NO: 51)
QVQLVQSGAEVAKPGSSVKVSCKASGYTFTDYTVNWVRQRPGQGLEWMGYI
NPFNSYTKYNQKFKDRVTITADESTSTAYMELSSLTSEDTAVYYCANSVSTY
WGQGTLVTVSS,
(SEQ ID NO: 52)
QVQLVQSGAEVAKPGSSVKVSCKTSGYTFTDYTVNWVRQRPGQGLEWMGYI
NPFNSYTKYNQKFKDRVTLTADESTSTAYMELSSLTSEDTAVYYCANSVSTY
WGQGTLVTVSS,
(SEQ ID NO: 53)
QVQLVQSGAEVAKPGSSVKVSCKTSGYTFTDYTVNWVRQRPGQGLEWMGYI
NPFNSYTKYNQKFKDRVTLTADKSTSTAYMQLSSLTSEDTAVYYCANSVSTY
WGQGTLVTVSS,
(SEQ ID NO: 54)
QVQLVQSGAEVKKPGASVKVSCKASGYTFTDYTVNWVRQAPGQRLEWMGY
INPFNSYTKYNQKFKDRVTITRDTSASTAYMELSSLRSEDTAVYYCARSVST
YWGQGTTVTVSS,
(SEQ ID NO: 55)
QVQLVQSGAEVKKPGASVKVSCKASGYTFTDYTVNWVRQAPGQRLEWIGYI
NPFNSYTKYNQKFKDRVTITADTSASTAYMELSSLRSEDTAVYYCANSVSTY
WGQGTTVTVSS,
(SEQ ID NO: 56)
QVQLVQSGAEVKKPGASVKVSCKASGYTFTDYTVNWVRQAPGQRLEWIGYI
NPFNSYTKYNQKFKDRATITADTSASTAYMELSSLRSEDTAVYFCANSVSTY
WGQGTTVTVSS,
(SEQ ID NO: 57)
QVQLVQSGAEVKKPGASVKVSCKASGYTFTDYTVNWVKQAPGQRLEWIGYI
NPFNSYTKYNQKFKDRATITADTSASTAYMELSSLRSEDTAVYFCANSVSTY
WGQGTTVTVSS,

or a sequence that is at least 85% identical to the foregoing sequences.

In some embodiments, the antibody or antigen-binding fragment thereof comprises a light chain variable region comprising the following sequence:

(SEQ ID NO: 58)
DIQMTQSPSSLSASVGDRVTITCKSSQSLLNNRTRKNYLAWYQQKPGKAPKLL
IYWASTRESGVPSRFSGSGSGTDFTLTISSLQPEDFATYYCKQSYTLRTFGGGTK
VEIK,
(SEQ ID NO: 59)
DIVMTQSPSSLSASVGDRVTITCKSSQSLLNQRTRKNYLAWYQQKPGKAPKLL
IYWASTRESGVPSRFSGSGSGTDFTLTISSLQPEDFATYYCKQSYTLRTFGGGTK
VEIK,
(SEQ ID NO: 60)
DIQMTQSPSSLSASVGDRVTMTCKSSQSLLNNRTRKNYLAWYQQKPGKAPKL
LIYWASTRESGVPSRFSGSGSGTDFTLTISSLQPEDFATYYCKQSYTLRTFGGGT
KVEIK,
(SEQ ID NO: 61)
DIVMTQSPSSLSASVGDRVTMTCKSSQSLLNQRTRKNYLAWYQQKPGKAPKL
LIYWASTRESGVPSRFTGSGSGTDFTLTISSLQPEDFATYYCKQSYTLRTFGGGT
KVEIK,
(SEQ ID NO: 62)
DIVMTQSPDSLAVSLGERATINCKSSQSLLNNRTRKNYLAWYQQKPGQPPKLL
IYWASTRESGVPDRFSGSGSGTDFTLTISSLQAEDVAVYYCKQSYTLRTFGGGT
KLEIK,
(SEQ ID NO: 63)
DIVMTQSPDSLAVSLGERATINCKSSQSLLNNRTRKNYLAWYQQKPGQPPKLL
IYWASTRESGVPDRFTGSGSGTDFTLTISSLQAEDVAVYYCKQSYTLRTFGGGT
KLEIK,
(SEQ ID NO: 64)
DIVMTQSPDSLAVSLGERATMSCKSSQSLLNNRTRKNYLAWYQQKPGQPPKL
LIYWASTRESGVPDRFSGSGSGTDFTLTISSLQAEDVAVYYCKQSYTLRTFGGG
TKLEIK,
(SEQ ID NO: 65)
DIVMTQSPDSLAVSLGERATMSCKSSQSLLNNRTRKNYLAWYQQKPGQPPKL
LIYWASTRESGVPDRFTGSGSGTDFTLTISSLQAEDVAVYYCKQSYTLRTFGGG
TKLEIK,
(SEQ ID NO: 66)
DIQMTQSPSSLSASVGDRVTITCKSSQSLLNSRTRKNYLAWYQQKPGKAPKLL
IYWASTRESGVPSRFSGSGSGTDFTLTISSLQPEDFATYYCKQSFILRTFGGGTK
VEIK,
(SEQ ID NO: 67)
DIQMTQSPSSLSASVGDRVTITCKSSQSLLNSRTRKNYLAWYQQKPGKSPKLLI
YWASTRESGVPDRFTGSGSGTDFTLTISSLQPEDFATYYCKQSFILRTFGGGTK
VAIK,
(SEQ ID NO: 68)
DIQMSQSPSSLSASVGDRVTITCKSSQSLLNSRTRKNYLAWYQQKPGQSPKLLI
YWASTRESGVPDRFTGSGSGTDFTLTISSLQAEDFATYYCKQSFILRTFGGGTK
VAIK,
(SEQ ID NO: 69)
DIVMSQSPSSLSASVGDRVTITCKSSQSLLNSRTRKNYLAWYQQKPGQSPKLLI
YWASTRESGVPDRFTGSGSGTDFTLTISSVQPEDFATYYCKQSFILRTFGGGTK
LAIK,
(SEQ ID NO: 70)
DIVMTQSPDSLAVSLGERATINCKSSQSLLNSRTRKNYLAWYQQKPGQPPKLLI
YWASTRESGVPDRFSGSGSGTDFTLTISSLQAEDVAVYYCKQSFILRTFGGGTK
LEIK,
(SEQ ID NO: 71)
DIVMTQSPDSLAVSLGERATINCKSSQSLLNSRTRKNYLAWYQQKPGQPPKLLI
YWASTRESGVPDRFTGSGSGTDFTLTISSLQAEDVAVYYCKQSFILRTFGGGTK
LEIK,
(SEQ ID NO: 72)
DIVMTQSPDSLAVSLGERATMNCKSSQSLLNSRTRKNYLAWYQQKPGQPPKL
LIYWASTRESGVPDRFSGSGSGTDFTLTISSLQAEDVAVYYCKQSFILRTFGGGT
KLEIK,
(SEQ ID NO: 73)
DIVMTQSPDSLAVSLGERATMNCKSSQSLLNSRTRKNYLAWYQQKPGQPPKL
LIYWASTRESGVPDRFTGSGSGTDFTLTISSLQAEDVAVYYCKQSFILRTFGGGT
KLEIK,
(SEQ ID NO: 74)
DVVMTQSPLSLPVTLGQPASISCKSSQSLLDSDGKTYLNWFQQRPGQSPRRLI
YLVSKLNSGVPDRFSGSGSGTDFTLKISRVEAEDVGVYYCWQGTHFPRTFGG
GTKVEIK,
(SEQ ID NO: 75)
DVVMTQSPLSLPVTLGQPASISCKSSQSLLDSDGKTYLNWLQQRPGQSPRRLI
YLVSKLNSGVPDRFSGSGSGTDFTLKISRVEAEDVGVYYCWQGTHFPRTFGG
GTKVEIK,
(SEQ ID NO: 76)
DVVMTQSPLSLPVTLGQPASISCKSSQSLLDSDGKTYLNWLQQRPGQSPRRLI
QLVSKLNSGVPDRFSGSGSGTDFTLKISRVEAEDVGVYYCWQGTHFPRTFGG
GTKVEIK,
(SEQ ID NO: 77)
DVVMTQSPLSLPVTLGQPASISCKSSQSLLDSDGKTYLNWFQQRPGQSPRRLI
YLVSKLNSGVPDRFSGSGSGTDFTLKISRVEAEDVGVYYCWQGTHFPRTFGG
GTKLEIK,
(SEQ ID NO: 78)
DVVMTQSPLSLPVTLGQPASISCKSSQSLLDSDGKTYLNWFQQRPGQSPRRLI
QLVSKLNSGVPDRFSGSGSGTDFTLKISRVEAEDVGVFYCWQGTHFPRTFGGG
TKLEIK,
(SEQ ID NO: 79)
DVVMTQSPLSLPVTLGQPASISCKSSQSLLDSDGKTYLNWLQQRPGQSPRRLI
QLVSKLNSGVPDRFSGSGSGTDFTLKISRVEAEDVGVFYCWQGTHFPRTFGGG
TKLEIK,
(SEQ ID NO: 80)
DVVMTQSPLSLPVTLGQPASISCKSSQSLLDSDGKTYLNWLLQRPGQSPRRLI
QLVSKLNSGVPDRFSGSGSGTDFTLKISRVEAEDVGVFYCWQGTHFPRTFGGG
TKLEIK,
(SEQ ID NO: 81)
DVVMTQTPLTLSVTIGQPASISCKSSQSLLDSDAKTYLNWLLQRPGQSPKRLIQ
LVSKLNSGVPDRFTGSGSGTDFTLKISRVEAEDLGIFYCWQGTHFPRTFGGGT
KLEIK,
(SEQ ID NO: 82)
DVVMTQTPLTLSVTIGQPASISCKSSQSLLDADGKTYLNWLLQRPGQSPKRLIQ
LVSKLNSGVPDRFTGSGSGTDFTLKISRVEAEDLGIYYCWQGTHFPRTFGGGT
KLEIK

or a sequence that is at least 85% identical to the aforementioned sequences.

In some embodiments, the antibody or antigen-binding fragment thereof comprises the following set of heavy chain variable regions and light chain variable regions:
(1) a heavy chain variable region of an amino acid sequence selected from SEQ ID NO: 33, SEQ ID NO: 34, SEQ ID NO: 35 or SEQ ID NO: 36; and a light chain variable region selected from SEQ ID NO: 58, SEQ ID NO: 59, SEQ ID NO: 60 or SEQ ID NO: 61; or
(2) a heavy chain variable region of an amino acid sequence selected from SEQ ID NO: 37, SEQ ID NO: 38, SEQ ID NO: 39 or SEQ ID NO: 40; and a light chain variable region of an amino acid sequence selected from SEQ ID NO: 62, SEQ ID NO: 63, SEQ ID NO: 64 or SEQ ID NO: 65; or
(3) a heavy chain variable region of an amino acid sequence selected from SEQ ID NO: 41, SEQ ID NO: 42, SEQ ID NO: 43 or SEQ ID NO: 44; and a light chain variable region of an amino acid sequence selected from SEQ ID NO: 66, SEQ ID NO: 67, SEQ ID NO: 68 or SEQ ID NO: 69; or
(4) a heavy chain variable region of an amino acid sequence selected from SEQ ID NO: 45, SEQ ID NO: 46, SEQ ID NO: 47 or SEQ ID NO: 48; and a light chain variable region of an amino acid sequence selected from SEQ ID NO: 70, SEQ ID NO: 71, SEQ ID NO: 72 or SEQ ID NO: 73; or
(5) a heavy chain variable region of an amino acid sequence selected from SEQ ID NO: 49, SEQ ID NO: 50, SEQ ID NO: 51, SEQ ID NO: 52 or SEQ ID NO: 53; and a light chain variable region of an amino acid sequence selected from SEQ ID NO: 74, SEQ ID NO: 75 or SEQ ID NO: 76; or
(6) a heavy chain variable region of an amino acid sequence selected from SEQ ID NO: 54, SEQ ID NO: 55, SEQ ID NO: 56 or SEQ ID NO: 57; and a light chain variable region of an amino acid sequence selected from SEQ ID NO: 77, SEQ ID NO: 78, SEQ ID NO: 79 or SEQ ID NO: 80.

In some embodiments, the antibody or antigen-binding fragment thereof comprises the following set of the heavy chain variable region and the light chain variable region of any one of (1) to (18):
(1) a heavy chain variable region having an amino acid sequence of SEQ ID NO: 38, and a light chain variable region having an amino acid sequence of SEQ ID NO: 62; or
(2) a heavy chain variable region having an amino acid sequence of SEQ ID NO: 38 and a light chain variable region having an amino acid sequence of SEQ ID NO: 63; or
(3) a heavy chain variable region having an amino acid sequence of SEQ ID NO: 38 and a light chain variable region having an amino acid sequence of SEQ ID NO: 64; or
(4) a heavy chain variable region having an amino acid sequence of SEQ ID NO: 39 and a light chain variable region having an amino acid sequence of SEQ ID NO: 63; or
(5) a heavy chain variable region having an amino acid sequence of SEQ ID NO: 39, and a light chain variable region having an amino acid sequence of SEQ ID NO: 64; or
(6) a heavy chain variable region having an amino acid sequence of SEQ ID NO: 39 and a light chain variable region having an amino acid sequence of SEQ ID NO: 65; or
(7) a heavy chain variable region having an amino acid sequence of SEQ ID NO: 46, and a light chain variable region having an amino acid sequence of SEQ ID NO: 71;
or
(8) a heavy chain variable region having an amino acid sequence of SEQ ID NO: 46, and a light chain variable region having an amino acid sequence of SEQ ID NO: 72; or
(9) a heavy chain variable region having an amino acid sequence of SEQ ID NO: 47 and a light chain variable region having an amino acid sequence of SEQ ID NO: 70; or
(10) a heavy chain variable region having an amino acid sequence of SEQ ID NO: 47 and a light chain variable region having an amino acid sequence of SEQ ID NO: 72; or
(11) a heavy chain variable region having an amino acid sequence of SEQ ID NO: 48 and a light chain variable region having an amino acid sequence of SEQ ID NO: 70; or
(12) a heavy chain variable region having an amino acid sequence of SEQ ID NO: 48 and a light chain variable region having an amino acid sequence of SEQ ID NO: 71; or
(13) a heavy chain variable region having an amino acid sequence of SEQ ID NO: 50 and a light chain variable region having an amino acid sequence of SEQ ID NO: 76; or
(14) a heavy chain variable region having an amino acid sequence of SEQ ID NO: 55 and a light chain variable region having an amino acid sequence of SEQ ID NO: 79; or
(15) a heavy chain variable region having an amino acid sequence of SEQ ID NO: 55, and a light chain variable region having an amino acid sequence of SEQ ID NO: 80; or
(16) a heavy chain variable region having an amino acid sequence of SEQ ID NO: 56, and a light chain variable region having an amino acid sequence of SEQ ID NO: 79; or
(17) a heavy chain variable region having an amino acid sequence of SEQ ID NO: 56, and a light chain variable region having an amino acid sequence of SEQ ID NO: 80; or
(18) a heavy chain variable region having an amino acid sequence of SEQ ID NO: 57, and a light chain variable region having an amino acid sequence of SEQ ID NO: 79.

In some embodiments, the antibody or antigen-binding fragment thereof is a human antibody, a humanized antibody, a chimeric antibody, a monoclonal antibody, a polyclonal antibody, a recombinant antibody, a single-chain antibody, a diabody, a three-chain antibody, a four-chain antibody, a Fab fragment, a F(ab′)₂ fragment, a scFv fragment, a Fv fragment, a Fab′ fragment, a domain antibody. In some embodiments, the antibody or antigen-binding fragment thereof is an IgA antibody, an IgD antibody, an IgE antibody, an IgM antibody, an IgG1 antibody, an IgG2 antibody, an IgG3 antibody, or an IgG4 antibody.

In some embodiments, the antibody or antigen-binding fragment thereof binds to human CD3 and/or cynomolgus monkey CD3.

In some embodiments, the antibody or antigen-binding fragment thereof binds to CD3e/d and/or CD3e/g.

In some embodiments, the antibody or antigen-binding fragment thereof (i) significantly activates total human T cells, upregulates CD25 expression and/or induces the release of IFN-γ; and/or (ii) binds to human CD3 with a K_{D} value of less than 4×10⁻⁸ M.

In a second aspect, the present disclosure relates to an antibody or an antigen-binding fragment thereof, which competes for or cross-blocks the binding to CD3, especially CD3e/d or CD3e/g, with the antibody or antigen-binding fragment as described in the first aspect.

In a third aspect, the present disclosure relates to an isolated nucleic acid encoding the antibody or antigen-binding fragment thereof as described in the first aspect or the second aspect.

In a fourth aspect, the present disclosure relates to a nucleic acid vector comprising the isolated nucleic acid as described in the third aspect.

In a fifth aspect, the present disclosure relates to a host cell comprising the isolated nucleic acid as described in the third aspect or the nucleic acid vector as described in the fourth aspect.

In a sixth aspect, the present disclosure relates to a pharmaceutical composition comprising the antibody or antigen-binding fragment thereof as described in the first aspect or the second aspect, the isolated nucleic acid as described in the third aspect or the nucleic acid vector as described in the fourth aspect, and a pharmaceutically acceptable carrier.

In a seventh aspect, the present disclosure relates to a method for producing the antibody or antigen-binding fragment thereof as described in the first aspect or the second aspect, comprising incubating the host cell as described in the fifth aspect under conditions that allow it to express the antibody or antigen-binding fragment thereof.

In an eighth aspect, the present disclosure relates to the use of the antibody or antigen-binding fragment thereof as described in any one of the first aspect or the second aspect, the isolated nucleic acid as described in the third aspect, the nucleic acid vector as described in the fourth aspect, or the pharmaceutical composition as described in the sixth aspect in the preparation of a medicament for suppressing an immune response or activating T cells.

In a ninth aspect, the present disclosure relates to the use of the antibody or antigen-binding fragment thereof as described in any one of the first aspect or the second aspect, the isolated nucleic acid as described in the third aspect, the nucleic acid vector as described in the fourth aspect, or the pharmaceutical composition as described in the sixth aspect in the preparation of a medicament for treating cancer or an autoimmune disease.

In a tenth aspect, the present disclosure relates to the antibody or antigen-binding fragment thereof as described in any one of the first aspect or the second aspect, the isolated nucleic acid as described in the third aspect, the nucleic acid vector as described in the fourth aspect, or the pharmaceutical composition as described in the sixth aspect, for use in suppressing an immune response or activating T cells, or for use in treating cancer or an autoimmune disease.

In an eleventh aspect, the present disclosure relates to a method for suppressing an immune response or activating T cells, or treating cancer or an autoimmune disease, comprising administering to an individual in need thereof a suppressing effective amount, an activating effective amount, or a therapeutically effective amount of the antibody or antigen-binding fragment thereof as described in any one of the first aspect or the second aspect, the isolated nucleic acid as described in the third aspect, the nucleic acid vector as described in the fourth aspect, or the pharmaceutical composition as described in the sixth aspect.

In some embodiments, the cancer described above is selected from the group consisting of melanoma (e.g., metastatic malignant melanoma), renal cancer, prostate cancer, breast cancer, colorectal cancer, lung cancer, nasopharyngeal cancer, oral cancer, liver cancer, bone cancer, pancreatic cancer, skin cancer, head and neck cancer, uterine cancer, ovarian cancer, rectal cancer, stomach cancer, testicular cancer, fallopian tube cancer, endometrial cancer, cervical cancer, vaginal cancer, vulvar cancer, Hodgkin's lymphoma, non-Hodgkin's lymphoma, esophageal cancer, small intestine cancer, large intestine cancer, endocrine system cancer, thyroid cancer, parathyroid cancer, adrenal cancer, soft tissue sarcoma, urethral cancer, penile cancer, chronic or acute leukemia (including acute myeloid leukemia, chronic myeloid leukemia, acute lymphoblastic leukemia, chronic lymphocytic leukemia), bladder cancer, renal pelvis cancer, central nervous system tumor, glioma, spinal axis tumor, brain stem glioma, pituitary adenoma, Kaposi's sarcoma, epidermoid carcinoma, squamous cell carcinoma, T-cell lymphoma, and combinations thereof.

In some embodiments, the autoimmune disease described above is selected from the group consisting of rheumatoid arthritis, multiple sclerosis, Sjögren's syndrome, insulin-dependent diabetes mellitus, autoimmune thyroiditis, reactive arthritis, ankylosing spondylitis, scleroderma, polymyositis, dermatomyositis, psoriasis, Wegener's granulomatosis, Crohn's disease, ulcerative colitis, lupus such as systemic lupus erythematosus, atherosclerosis, chronic obstructive pulmonary disease, cirrhosis, renal transplant fibrosis, renal transplant nephropathy, pulmonary fibrosis, and combinations thereof.

These and other aspects are described in greater detail in the present disclosure. Each of the aspects provided can encompass various embodiments provided herein. It is therefore anticipated that each of the embodiments involving one element or combinations of elements can be included in each aspect described, and all such combinations of the above aspects and embodiments are expressly considered.

### BRIEF DESCRIPTION OF THE DRAWINGS

FIG. 1 is a graph showing the results of serum titers against immunogens in mouse sera as detected by ELISA.

FIG. 2 is a graph showing the results of the binding of CD3 antibodies to human CD3e/g as detected by ELISA.

FIG. 3 is a graph showing the results of the binding of CD3 antibodies to cynomolgus monkey CD3e/g as detected by ELISA.

FIG. 4 is a graph showing the results of the binding of CD3 antibodies to human CD3e/d as detected by ELISA.

FIG. 5 (a)-(e) are graphs showing the results of the binding of CD3 antibodies to Jurkat cells as detected by FACS.

FIG. 6 (a)-(h) are graphs showing the effect of CD3 antibodies on CD25 expression as detected by FACS.

FIG. 7 (a)-(f) are graphs showing the effect of CD3 antibodies on IFN-γ secretion as detected by FACS.

### DETAILED DESCRIPTION OF THE INVENTION

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present application are generally performed according to conventional methods well known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001), Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well known and commonly used in the art. Standard techniques can be used for chemical syntheses, chemical analyses, pharmaceutical preparation, formulation, and delivery, and treatment of patients.

It should be understood that the instant disclosure is not limited to the particular methodology, protocols, and reagents, etc., described herein and as such can vary. The terminology used herein is for the purpose of describing particular embodiments only, and is not intended to limit the scope of the present disclosure.

Other than in the operating examples, or where otherwise indicated, all numbers expressing quantities of ingredients or reaction conditions used herein should be understood as modified in all instances by the term “about”. The term “about” when used in connection with percentages can mean±5%, e.g., 1%, 2%, 3%, or 4%.

### Definition

The terms used herein are only for the purpose of describing specific embodiments and are not intended to limit the present invention. Unless the context clearly indicates otherwise, the open-ended expressions “include” and “comprise” are interpreted as also including unmentioned structural components or method steps, but it should be noted that the open-ended expressions also cover situations only consisting of the described components and method steps (i.e., covering situations of the closed-ended expression “consist of”).

As used throughout, a range is used as a shorthand for describing each and all numerical values within the range. Any numerical value, especially integer value within the range can be selected as the endpoint of the range. For example, the range “at least 80% identical” is used to describe all numerical values within the range, such as 80%, 81%, 82%, 83%, 84%, 85%, 86%, 87%, 88%, 89%, 90%, 91%, 92%, 93%, 94%, 95%, 96%, 97%, 98%, 99% and 100%, and includes all subranges, such as at least 85%, at least 90%, at least 95%, etc.

As used herein, an “antibody” refers to a monomer as well as a multimer. A whole antibody (including a multimer) or an antibody fragment carrying an antigen-binding fragment of the antibody can be used. The antibody can be a human antibody, a humanized antibody, a chimeric antibody, a monoclonal antibody, a polyclonal antibody or a recombinant antibody. An antigen-binding fragment can be produced by recombinant DNA technology or by enzymatic or chemical cleavage of a whole antibody.

A “Fab fragment” is a monovalent fragment having the V_{L}, V_{H}, C_{L} and C_{H} domains; a “F(ab′) 2 fragment” is a bivalent fragment having two Fab fragments linked by a disulfide bridge at the hinge region.

A single-chain antibody (scFv) is an antibody in which a V_{L} and a V_{H} region are joined via a linker (e.g., a synthetic sequence of amino acid residues) to form a continuous protein chain wherein the linker is long enough to allow the protein chain to fold back on itself and form a monovalent antigen binding site (see, e.g., Bird et al., Science 242:423-26 (1988) and Huston et al., 1988, Proc. Natl. Acad. Sci. USA 85:5879-83 (1988)). Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48 (1993), and Poljak et al., Structure 2:1121-23 (1994)). If the two polypeptide chains of a diabody are identical, then a diabody resulting from their pairing will have two identical antigen binding sites. Polypeptide chains having different sequences can be used to make a diabody with two different antigen binding sites. Similarly, tribodies (or “three-chain antibodies”) and tetrabodies (or “four-chain antibodies”) are antibodies comprising three and four polypeptide chains, respectively, and forming three and four antigen binding sites, respectively, which can be the same or different.

Complementarity determining regions (CDRs) and framework regions (FR) of a given antibody can be identified using the system described by Kabat et al. in Sequences of Proteins of Immunological Interest, 5th Ed., US Dept. of Health and Human Services, PHS, NIH, NIH Publication no. 91-3242, 1991. As desired, the CDRs can also be redefined according an alternative nomenclature scheme, such as that of Chothia (see Chothia & Lesk, 1987, J. Mol. Biol. 196:901-917; Chothia et al., 1989, Nature 342:878-883 or Honegger & Pluckthun, 2001, J. Mol. Biol. 309:657-670. One or more CDRs can be incorporated into a molecule either covalently or noncovalently to make it an antigen binding protein, such as an antibody. An antigen binding protein can incorporate the CDR(s) as part of a larger polypeptide chain, can covalently link the CDR(s) to another polypeptide chain, or can incorporate the CDR(s) noncovalently. The CDRs permit the antigen binding protein to specifically bind to a particular antigen of interest.

In the art, CDRs of an antibody can be defined by a variety of methods, and the methods and techniques for identifying the variable region in an antibody molecule and the CDRs in the amino acid sequence of the variable region of the antibody are also well known in the art, and can be used to identify the CDRs in the amino acid sequences of the variable regions of specific antibodies disclosed herein. In the present application, the amino acid sequences of the recited CDRs are all shown according to the Kabat definition rule (sequences in the claims of the present application are also shown according to the Kabat definition rule). It should be understood by those skilled in the art that, unless otherwise specified, the terms “CDRs” and “complementarity determining regions” of a given antibody or its regions (e.g., variable regions) should be understood to cover the complementary determining regions defined by any of the above-mentioned known schemes described in the present disclosure. Although the scope of protection claimed in the present disclosure is based on the sequences shown in the Kabat definition rule, the amino acid sequences defined according to other CDR definition rules should also fall within the scope of protection of the present invention.

An “antibody or antigen-binding fragment thereof” described herein may also include an antibody heavy chain constant region and/or an antibody light chain constant region. For example, the antibody heavy chain constant region is a mouse antibody heavy chain constant region or a human antibody heavy chain constant region; the antibody light chain constant region is a mouse light chain antibody constant region or a human antibody light chain constant region. More preferably, the antibody heavy chain constant region is a human antibody heavy chain constant region, such as a human IgG1, IgG2a, IgG2b, IgG2c, IgG3 or IgG4 antibody heavy chain constant region; the antibody light chain constant region is a human antibody light chain k or A chain constant region.

The term “humanized antibody” includes antibodies having variable and constant regions of human germline immunoglobulin sequences wherein CDR sequences derived from another germline have been grafted onto human framework sequences.

The term “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′, 3′-dideoxyribose, and internucleotide linkage modifications such as phosphorothioate, phosphorodithioate, phosphoroselenoate, phosphorodiselenoate, phosphoroanilothioate, and phosphoroamidate.

The term “vector” means any molecule or entity (e.g., nucleic acid, plasmid, bacteriophage or virus) used to transfer protein coding information into a host cell.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, as long as the gene of interest is present. The host cell can be a prokaryote (for example, Escherichia coli), or can be a eukaryote (for example, a unicellular eukaryote (for example, yeast or other fungi), a vegetable cell (for example, tobacco or tomato plant cell), an animal cell (for example, a human cell, a monkey cell, a hamster cell, a rat cell, a mouse cell, or an insect cell)) or a hybridoma.

The term “identity” refers to the relationship between the sequences of two or more polypeptide molecules or two or more nucleic acid molecules, as determined by alignment and comparison of sequences. Generally, identity refers to the number or percentage of identical positions shared by two amino acid or nucleic acid sequences, taking into account the number of gaps that need to be introduced to achieve optimal alignment of the two sequences and the length of each gap. When an amino acid sequence is described as being at least 85% or at least 90% or at least 95% identical to another amino acid sequence, the difference in the amino acid sequence may be conservative substitutions (including where all substitutions are conservative substitutions).

As used herein, “conservative substitution” is considered in the art to replace an amino acid with another amino acid having similar properties. Exemplary conservative substitutions are well known in the art (see, e.g., WO 97/09433, p. 10, published on Mar. 13, 1997; Lehninger, Biochemistry, 2nd ed.; Worth Publishers, Inc. NY: NY (1975), p. 71-77; Lewin, Genes IV, Oxford University Press, NY and Cell Press, Cambridge, MA (1990), p. 8).

Naturally-occurring amino acids can be divided into classes based on common side chain properties:
1) hydrophobic: norleucine (Nle), Met, Ala, Val, Leu, Ile;
2) neutral hydrophilic: Cys, Ser, Thr, Asn, Gln;
3) acidic: Asp, Glu;
4) basic: His, Lys, Arg;
5) residues that influence chain orientation: Gly, Pro; and
6) aromatic: Trp, Tyr, Phe.

Conservative substitutions can involve exchange of a member of one of these classes with another member of the same class. Conservative amino acid substitutions can encompass non-naturally occurring amino acid residues, which are typically incorporated by chemical peptide synthesis rather than by synthesis in biological systems. These include peptidomimetics and other reversed or inverted forms of amino acid moieties. Non-conservative substitutions can involve the exchange of a member of one of the above classes for a member from another class. Such substituted residues can be introduced into regions of the antibody that are homologous with human antibodies, or into the non-homologous regions of the molecule.

An “effective amount” is generally an amount sufficient to reduce the severity and/or frequency of symptoms, eliminate symptoms and/or underlying cause, prevent the occurrence of symptoms and/or their underlying cause, and/or improve or remediate the damage that results from or is associated with a disease (e.g., cancer or an autoimmune disease). In some embodiments, the effective amount is a therapeutically effective amount or a prophylactically effective amount. A “therapeutically effective amount” is an amount sufficient to remedy a disease state (e.g., cancer or an autoimmune disease) or symptoms, particularly a state or symptoms associated with the disease state, or otherwise prevent, hinder, retard or reverse the progression of the disease state or any other undesirable symptom associated with the disease in any way whatsoever. The therapeutically effective amount of the antibody or antigen-binding protein thereof of the disclosure, the nucleic acid encoding the antibody or antigen-binding fragment, the nucleic acid vector comprising the nucleic acid, the host cell comprising the nucleic acid or nucleic acid vector, or the pharmaceutical composition comprising the aforementioned substances to be employed will depend, for example, upon the therapeutic context and objectives. One skilled in the art will appreciate that the appropriate dosage levels for treatment will vary depending, in part, upon the molecule delivered, the indication for which the antibody or the antigen binding protein thereof is being used, the route of administration, and the size (body weight, body surface or organ size) and/or condition (the age and general health) of the patient. In certain embodiments, the clinician can titer the dosage and modify the route of administration to obtain the optimal therapeutic effect.

The terms “subject”, “patient” and “individual” are used interchangeably herein and refer to warm-blooded animals, such as mammals. The terms include, but are not limited to, livestock, rodents (such as rats and mice), primates and humans. Preferably, the terms refer to humans.

As used herein, the term “EC₅₀” refers to the concentration for half of the maximal effect, i.e., the concentration that induces 50% of the maximal effect.

The terms “CD3 antibody”, “anti-CD3 antibody”, “antibody that specifically binds to CD3” are used interchangeably and refer to an antibody that can specifically bind to human or other mammalian CD3. In some embodiments, the antibody can specifically bind to the CD3 antigen with a K_{D} value of less than 1×10⁻⁴, less than 1×10⁻⁵, less than 1×10⁻⁶, less than 1×10⁻⁷, less than 1×10⁻⁸, less than 1×10⁻⁹, less than 1×10⁻¹⁰ or less than 1×10⁻¹¹ M. The term “CD3e/g antibody” refers to an antibody that can specifically bind to human or other mammalian CD3e/g subunit. In some embodiments, the CD3e/g antigen has a sequence as set forth in SEQ ID NO: 83 and/or SEQ ID NO: 84. In some embodiments, the antibody can specifically bind to the CD3e/g antigen with a K_{D} value of less than 1×10⁻⁴, less than 1×10⁻⁵, less than 1×10⁻⁶, less than 1×10⁻⁷, less than 1×10⁻⁸, less than 1×10⁻⁹, less than 1×10⁻¹⁰ or less than 1×10⁻¹¹ M. The term “CD3e/d antibody” refers to an antibody that can specifically bind to human or other mammalian CD3e/d subunit. In some embodiments, the antibody can specifically bind to the CD3e/d antigen with a K_{D} value of less than 1×10⁻⁴, less than 1×10⁻⁵, less than 1×10⁻⁶, less than 1×10⁻⁷, less than 1×10⁻⁸, less than 1×10⁻⁹, less than 1×10⁻¹⁰ or less than 1×10⁻¹¹ M. In some preferred embodiments, the antibody can specifically bind to the CD3e/g antigen and/or CD3e/d antigen with a K_{D} value of less than 1×10⁻⁶, less than 1×10⁻⁷, less than 1×10⁻⁸, less than 1×10⁻⁹, less than 1×10⁻¹⁰ or less than 1×10⁻¹¹ M.

The term “K_{D}” is the equilibrium dissociation constant, which is a common parameter for characterizing antibody affinity. Its calculation formula is K_{D}=[antibody][antigen]/[antibody-antigen], which represents the degree of dissociation of the antibody-antigen complex at equilibrium. The larger the K_{D} is, the more dissociation there is, which means the weaker the affinity between the antibody and the antigen is. The smaller the K_{D} is, the less dissociation there is, which means the stronger the affinity between the antibody and the antigen is. In some preferred embodiments, the above-mentioned antibody binds to human CD3 with a K_{D} value of less than 4×10⁻⁸ M.

As used herein, in an expression similar to “significantly activates total T cells, upregulates CD25 expression and/or induces the release of IFN-γ”, “significantly” can mean that at least about 50%, at least about 60%, at least about 70%, at least about 80%, at least about 85%, at least about 90% or at least about 95% of the cells are activated; or the expression of CD25 is upregulated by at least about 50%, at least about 60%, at least about 70%, at least about 80%, at least about 85%, at least about 90% or at least about 95%; or the release amount of IFN-γ as induced is at least about 5000 μg/mL, about 6000 μg/mL, about 7000 μg/mL, about 8000 μg/mL, about 9000 μg/mL, about 10000 μg/mL, about 11000 μg/mL, about 12000 μg/mL, about 13000 μg/mL, about 14000 μg/mL, about 15000 μg/mL, about 16000 μg/mL, about 17000 μg/mL, about 18000 μg/mL, about 19000 μg/mL or about 20000 μg/mL. In some embodiments, “significantly” can refer to activating total T cells, upregulating CD25 expression, and/or inducing the release of IFN-γ to an amount, which is at least about 1.5 times, at least about 2 times, at least about 2.5 times, at least about 3 times, at least about 3.5 times, at least about 4 times or more higher than that of the negative control, i.e., isotype control or blank control. In some preferred embodiments, “significantly” can refer to activating total T cells, upregulating CD25 expression, and/or inducing the release of IFN-γ to an amount, which is at least about 3 times higher than that of the negative control, i.e., isotype control or blank control.

The term “isotype control” refers to an antibody that has similar properties to the test antibody but lacks its specific target. In flow cytometry experiments, an isotype control serves as a negative control that can distinguish signals from a specific antibody from nonspecific background staining, eliminating interfering factors to ensure the accuracy of experimental results.

### Antibody

The present disclosure relates to an antibody or antigen-binding fragment thereof that specifically binds to CD3 (e.g., CD3e, in particular CD3e/d and/or CD3e/g). In some embodiments, the CD3 is human CD3 and/or cynomolgus monkey CD3.

In general, the antibody or antigen-binding fragment thereof of the present disclosure generally comprises one or more CDRs (e.g., 1, 2, 3, 4, 5, or 6 CDRs) as described herein.

In some embodiments, the heavy chain CDR1 comprises a sequence that has at least 80% identity (e.g., at least 85%, 90%, 95%, 96%, 97%, 98%, 99% or 100% identity) to any of SEQ ID NOs: 1-4, or a sequence that differs from any of SEQ ID NOs: 1-4 by no more than a total of 3 (e.g., 1, 2, and 3) amino acid additions, substitutions and/or deletions. In some embodiments, the heavy chain CDR2 comprises a sequence that has at least 80% identity (e.g., at least 85%, 90%, 95%, 96%, 97%, 98%, 99% or 100% identity) to any of SEQ ID NOs: 5-7 and 18-23, or a sequence that differs from any of SEQ ID NOs: 5-7 and 18-23 by no more than a total of 3 (e.g., 1, 2, and 3) amino acid additions, substitutions and/or deletions. In some embodiments, the heavy chain CDR3 comprises a sequence that has at least 80% identity (e.g., at least 85%, 90%, 95%, 96%, 97%, 98%, 99% or 100% identity) to any of SEQ ID NOs: 8-11, or a sequence that differs from any of SEQ ID NOs: 8-11 by no more than a total of 3 (e.g., 1, 2, and 3) amino acid additions, substitutions and/or deletions.

In some embodiments, the light chain CDR1 comprises a sequence that has at least 80% identity (e.g., at least 85%, 90%, 95%, 96%, 97%, 98%, 99% or 100% identity) to any of SEQ ID NOs: 12-13 and 24-30, or a sequence that differs from any of SEQ ID NOs: 12-13 and 24-30 by no more than a total of 3 (e.g., 1, 2, and 3) amino acid additions, substitutions and/or deletions. In some embodiments, the light chain CDR2 comprises a sequence that has at least 80% identity (e.g., at least 85%, 90%, 95%, 96%, 97%, 98%, 99% or 100% identity) to any of SEQ ID NOs: 14-15, or a sequence that differs from any of SEQ ID NOs: 14-15 by no more than a total of 3 (e.g., 1, 2, and 3) amino acid additions, substitutions and/or deletions. In some embodiments, the light chain CDR3 comprises a sequence that has at least 80% identity (e.g., at least 85%, 90%, 95%, 96%, 97%, 98%, 99% or 100% identity) to any of SEQ ID NOs: 16-17 and 31-32, or a sequence that differs from any of SEQ ID NOs: 16-17 and 31-32 by no more than a total of 3 (e.g., 1, 2, and 3) amino acid additions, substitutions and/or deletions.

In some embodiments, the substitutions are conservative substitutions.

In some embodiments, the antibody or antigen-binding fragment thereof in the present disclosure comprises a heavy chain variable region and/or a light chain variable region. In some embodiments, the heavy chain variable region comprises an amino acid sequence selected from SEQ ID NOs: 33-57 or a sequence having at least 85% identity (e.g., at least 90%, 95%, 96%, 97%, 98%, 99% or 100% identity) to any one of the aforementioned sequences. In some embodiments, the light chain variable region comprises an amino acid sequence selected from SEQ ID NOs: 58-82 or a sequence having at least 85% identity (e.g., at least 90%, 95%, 96%, 97%, 98%, 99% or 100% identity) to any one of the aforementioned sequences.

In some preferred embodiments, the antibody or antigen-binding fragment thereof has a heavy chain variable region as set forth in SEQ ID NO: 33 and a light chain variable region as set forth in SEQ ID NO: 58; in some preferred embodiments, the antibody or antigen-binding fragment thereof has a heavy chain variable region as set forth in SEQ ID NO: 33 and a light chain variable region as set forth in SEQ ID NO: 59; in some preferred embodiments, the antibody or antigen-binding fragment thereof has a heavy chain variable region as set forth in SEQ ID NO: 33 and a light chain variable region as set forth in SEQ ID NO: 60; in some preferred embodiments, the antibody or antigen-binding fragment thereof has a heavy chain variable region as set forth in SEQ ID NO: 33 and a light chain variable region as set forth in SEQ ID NO: 61. In some preferred embodiments, the antibody or antigen-binding fragment thereof has a heavy chain variable region as set forth in SEQ ID NO: 34 and a light chain variable region as set forth in SEQ ID NO: 58; in some preferred embodiments, the antibody or antigen-binding fragment thereof has a heavy chain variable region as set forth in SEQ ID NO: 34 and a light chain variable region as set forth in SEQ ID NO: 59; in some preferred embodiments, the antibody or antigen-binding fragment thereof has a heavy chain variable region as set forth in SEQ ID NO: 34 and a light chain variable region as set forth in SEQ ID NO: 60; in some preferred embodiments, the antibody or antigen-binding fragment thereof has a heavy chain variable region as set forth in SEQ ID NO: 34 and a light chain variable region as set forth in SEQ ID NO: 61. In some preferred embodiments, the antibody or antigen-binding fragment thereof has a heavy chain variable region as set forth in SEQ ID NO: 35 and a light chain variable region as set forth in SEQ ID NO: 58; in some preferred embodiments, the antibody or antigen-binding fragment thereof has a heavy chain variable region as set forth in SEQ ID NO: 35 and a light chain variable region as set forth in SEQ ID NO: 59; in some preferred embodiments, the antibody or antigen-binding fragment thereof has a heavy chain variable region as set forth in SEQ ID NO: 35 and a light chain variable region as set forth in SEQ ID NO: 60; in some preferred embodiments, the antibody or antigen-binding fragment thereof has a heavy chain variable region as set forth in SEQ ID NO: 35 and a light chain variable region as set forth in SEQ ID NO: 61. In some preferred embodiments, the antibody or antigen-binding fragment thereof has a heavy chain variable region as set forth in SEQ ID NO: 36 and a light chain variable region as set forth in SEQ ID NO: 58; in some preferred embodiments, the antibody or antigen-binding fragment thereof has a heavy chain variable region as set forth in SEQ ID NO: 36 and a light chain variable region as set forth in SEQ ID NO: 59; in some preferred embodiments, the antibody or antigen-binding fragment thereof has a heavy chain variable region as set forth in SEQ ID NO: 36 and a light chain variable region as set forth in SEQ ID NO: 60; in some preferred embodiments, the antibody or antigen-binding fragment thereof has a heavy chain variable region as set forth in SEQ ID NO: 36 and a light chain variable region as set forth in SEQ ID NO: 61. In some preferred embodiments, the antibody or antigen-binding fragment thereof has a heavy chain variable region as set forth in SEQ ID NO: 37 and a light chain variable region as set forth in SEQ ID NO: 62; in some preferred embodiments, the antibody or antigen-binding fragment thereof has a heavy chain variable region as set forth in SEQ ID NO: 37 and a light chain variable region as set forth in SEQ ID NO: 63; in some preferred embodiments, the antibody or antigen-binding fragment thereof has a heavy chain variable region as set forth in SEQ ID NO: 37 and a light chain variable region as set forth in SEQ ID NO: 64; in some preferred embodiments, the antibody or antigen-binding fragment thereof has a heavy chain variable region as set forth in SEQ ID NO: 37 and a light chain variable region as set forth in SEQ ID NO: 65. In some preferred embodiments, the antibody or antigen-binding fragment thereof has a heavy chain variable region as set forth in SEQ ID NO: 38 and a light chain variable region as set forth in SEQ ID NO: 62; in some preferred embodiments, the antibody or antigen-binding fragment thereof has a heavy chain variable region as set forth in SEQ ID NO: 38 and a light chain variable region as set forth in SEQ ID NO: 63; in some preferred embodiments, the antibody or antigen-binding fragment thereof has a heavy chain variable region as set forth in SEQ ID NO: 38 and a light chain variable region as set forth in SEQ ID NO: 64; in some preferred embodiments, the antibody or antigen-binding fragment thereof has a heavy chain variable region as set forth in SEQ ID NO: 38 and a light chain variable region as set forth in SEQ ID NO: 65. In some preferred embodiments, the antibody or antigen-binding fragment thereof has a heavy chain variable region as set forth in SEQ ID NO: 39 and a light chain variable region as set forth in SEQ ID NO: 62; in some preferred embodiments, the antibody or antigen-binding fragment thereof has a heavy chain variable region as set forth in SEQ ID NO: 39 and a light chain variable region as set forth in SEQ ID NO: 63; in some preferred embodiments, the antibody or antigen-binding fragment thereof has a heavy chain variable region as set forth in SEQ ID NO: 39 and a light chain variable region as set forth in SEQ ID NO: 64; in some preferred embodiments, the antibody or antigen-binding fragment thereof has a heavy chain variable region as set forth in SEQ ID NO: 39 and a light chain variable region as set forth in SEQ ID NO: 65. In some preferred embodiments, the antibody or antigen-binding fragment thereof has a heavy chain variable region as set forth in SEQ ID NO: 40 and a light chain variable region as set forth in SEQ ID NO: 62; in some preferred embodiments, the antibody or antigen-binding fragment thereof has a heavy chain variable region as set forth in SEQ ID NO: 40 and a light chain variable region as set forth in SEQ ID NO: 63; in some preferred embodiments, the antibody or antigen-binding fragment thereof has a heavy chain variable region as set forth in SEQ ID NO: 40 and a light chain variable region as set forth in SEQ ID NO: 64; in some preferred embodiments, the antibody or antigen-binding fragment thereof has a heavy chain variable region as set forth in SEQ ID NO: 40 and a light chain variable region as set forth in SEQ ID NO: 65.

In some preferred embodiments, the antibody or antigen-binding fragment thereof has a heavy chain variable region as set forth in SEQ ID NO: 41 and a light chain variable region as set forth in SEQ ID NO: 66; in some preferred embodiments, the antibody or antigen-binding fragment thereof has a heavy chain variable region as set forth in SEQ ID NO: 41 and a light chain variable region as set forth in SEQ ID NO: 67; in some preferred embodiments, the antibody or antigen-binding fragment thereof has a heavy chain variable region as set forth in SEQ ID NO: 41 and a light chain variable region as set forth in SEQ ID NO: 68; in some preferred embodiments, the antibody or antigen-binding fragment thereof has a heavy chain variable region as set forth in SEQ ID NO: 41 and a light chain variable region as set forth in SEQ ID NO: 69. In some preferred embodiments, the antibody or antigen-binding fragment thereof has a heavy chain variable region as set forth in SEQ ID NO: 42 and a light chain variable region as set forth in SEQ ID NO: 66; in some preferred embodiments, the antibody or antigen-binding fragment thereof has a heavy chain variable region as set forth in SEQ ID NO: 42 and a light chain variable region as set forth in SEQ ID NO: 67; in some preferred embodiments, the antibody or antigen-binding fragment thereof has a heavy chain variable region as set forth in SEQ ID NO: 42 and a light chain variable region as set forth in SEQ ID NO: 68; in some preferred embodiments, the antibody or antigen-binding fragment thereof has a heavy chain variable region as set forth in SEQ ID NO: 42 and a light chain variable region as set forth in SEQ ID NO: 69. In some preferred embodiments, the antibody or antigen-binding fragment thereof has a heavy chain variable region as set forth in SEQ ID NO: 43 and a light chain variable region as set forth in SEQ ID NO: 66; in some preferred embodiments, the antibody or antigen-binding fragment thereof has a heavy chain variable region as set forth in SEQ ID NO: 43 and a light chain variable region as set forth in SEQ ID NO: 67; in some preferred embodiments, the antibody or antigen-binding fragment thereof has a heavy chain variable region as set forth in SEQ ID NO: 43 and a light chain variable region as set forth in SEQ ID NO: 68; in some preferred embodiments, the antibody or antigen-binding fragment thereof has a heavy chain variable region as set forth in SEQ ID NO: 43 and a light chain variable region as set forth in SEQ ID NO: 69. In some preferred embodiments, the antibody or antigen-binding fragment thereof has a heavy chain variable region as set forth in SEQ ID NO: 44 and a light chain variable region as set forth in SEQ ID NO: 66; in some preferred embodiments, the antibody or antigen-binding fragment thereof has a heavy chain variable region as set forth in SEQ ID NO: 44 and a light chain variable region as set forth in SEQ ID NO: 67; in some preferred embodiments, the antibody or antigen-binding fragment thereof has a heavy chain variable region as set forth in SEQ ID NO: 44 and a light chain variable region as set forth in SEQ ID NO: 68; in some preferred embodiments, the antibody or antigen-binding fragment thereof has a heavy chain variable region as set forth in SEQ ID NO: 44 and a light chain variable region as set forth in SEQ ID NO: 69.

In some preferred embodiments, the antibody or antigen-binding fragment thereof has a heavy chain variable region as set forth in SEQ ID NO: 45 and a light chain variable region as set forth in SEQ ID NO: 70; in some preferred embodiments, the antibody or antigen-binding fragment thereof has a heavy chain variable region as set forth in SEQ ID NO: 45 and a light chain variable region as set forth in SEQ ID NO: 71; in some preferred embodiments, the antibody or antigen-binding fragment thereof has a heavy chain variable region as set forth in SEQ ID NO: 45 and a light chain variable region as set forth in SEQ ID NO: 72; in some preferred embodiments, the antibody or antigen-binding fragment thereof has a heavy chain variable region as set forth in SEQ ID NO: 45 and a light chain variable region as set forth in SEQ ID NO: 73. In some preferred embodiments, the antibody or antigen-binding fragment thereof has a heavy chain variable region as set forth in SEQ ID NO: 46 and a light chain variable region as set forth in SEQ ID NO: 70; in some preferred embodiments, the antibody or antigen-binding fragment thereof has a heavy chain variable region as set forth in SEQ ID NO: 46 and a light chain variable region as set forth in SEQ ID NO: 71; in some preferred embodiments, the antibody or antigen-binding fragment thereof has a heavy chain variable region as set forth in SEQ ID NO: 46 and a light chain variable region as set forth in SEQ ID NO: 72; in some preferred embodiments, the antibody or antigen-binding fragment thereof has a heavy chain variable region as set forth in SEQ ID NO: 46 and a light chain variable region as set forth in SEQ ID NO: 73. In some preferred embodiments, the antibody or antigen-binding fragment thereof has a heavy chain variable region as set forth in SEQ ID NO: 47 and a light chain variable region as set forth in SEQ ID NO: 70; in some preferred embodiments, the antibody or antigen-binding fragment thereof has a heavy chain variable region as set forth in SEQ ID NO: 47 and a light chain variable region as set forth in SEQ ID NO: 71; in some preferred embodiments, the antibody or antigen-binding fragment thereof has a heavy chain variable region as set forth in SEQ ID NO: 47 and a light chain variable region as set forth in SEQ ID NO: 72; in some preferred embodiments, the antibody or antigen-binding fragment thereof has a heavy chain variable region as set forth in SEQ ID NO: 47 and a light chain variable region as set forth in SEQ ID NO: 73. In some preferred embodiments, the antibody or antigen-binding fragment thereof has a heavy chain variable region as set forth in SEQ ID NO: 48 and a light chain variable region as set forth in SEQ ID NO: 70; in some preferred embodiments, the antibody or antigen-binding fragment thereof has a heavy chain variable region as set forth in SEQ ID NO: 48 and a light chain variable region as set forth in SEQ ID NO: 71; in some preferred embodiments, the antibody or antigen-binding fragment thereof has a heavy chain variable region as set forth in SEQ ID NO: 48 and a light chain variable region as set forth in SEQ ID NO: 72; in some preferred embodiments, the antibody or antigen-binding fragment thereof has a heavy chain variable region as set forth in SEQ ID NO: 48 and a light chain variable region as set forth in SEQ ID NO: 73.

In some preferred embodiments, the antibody or antigen-binding fragment thereof has a heavy chain variable region as set forth in SEQ ID NO: 49 and a light chain variable region as set forth in SEQ ID NO: 74; in some preferred embodiments, the antibody or antigen-binding fragment thereof has a heavy chain variable region as set forth in SEQ ID NO: 49 and a light chain variable region as set forth in SEQ ID NO: 75; in some preferred embodiments, the antibody or antigen-binding fragment thereof has a heavy chain variable region as set forth in SEQ ID NO: 49 and a light chain variable region as set forth in SEQ ID NO: 76. In some preferred embodiments, the antibody or antigen-binding fragment thereof has a heavy chain variable region as set forth in SEQ ID NO: 50 and a light chain variable region as set forth in SEQ ID NO: 74; in some preferred embodiments, the antibody or antigen-binding fragment thereof has a heavy chain variable region as set forth in SEQ ID NO: 50 and a light chain variable region as set forth in SEQ ID NO: 75; in some preferred embodiments, the antibody or antigen-binding fragment thereof has a heavy chain variable region as set forth in SEQ ID NO: 50 and a light chain variable region as set forth in SEQ ID NO: 76. In some preferred embodiments, the antibody or antigen-binding fragment thereof has a heavy chain variable region as set forth in SEQ ID NO: 51 and a light chain variable region as set forth in SEQ ID NO: 74; in some preferred embodiments, the antibody or antigen-binding fragment thereof has a heavy chain variable region as set forth in SEQ ID NO: 51 and a light chain variable region as set forth in SEQ ID NO: 75; in some preferred embodiments, the antibody or antigen-binding fragment thereof has a heavy chain variable region as set forth in SEQ ID NO: 51 and a light chain variable region as set forth in SEQ ID NO: 76. In some preferred embodiments, the antibody or antigen-binding fragment thereof has a heavy chain variable region as set forth in SEQ ID NO: 52 and a light chain variable region as set forth in SEQ ID NO: 74; in some preferred embodiments, the antibody or antigen-binding fragment thereof has a heavy chain variable region as set forth in SEQ ID NO: 52 and a light chain variable region as set forth in SEQ ID NO: 75; in some preferred embodiments, the antibody or antigen-binding fragment thereof has a heavy chain variable region as set forth in SEQ ID NO: 52 and a light chain variable region as set forth in SEQ ID NO: 76. In some preferred embodiments, the antibody or antigen-binding fragment thereof has a heavy chain variable region as set forth in SEQ ID NO: 53 and a light chain variable region as set forth in SEQ ID NO: 74; in some preferred embodiments, the antibody or antigen-binding fragment thereof has a heavy chain variable region as set forth in SEQ ID NO: 53 and a light chain variable region as set forth in SEQ ID NO: 75; in some preferred embodiments, the antibody or antigen-binding fragment thereof has a heavy chain variable region as set forth in SEQ ID NO: 53 and a light chain variable region as set forth in SEQ ID NO: 76.

In some preferred embodiments, the antibody or antigen-binding fragment thereof has a heavy chain variable region as set forth in SEQ ID NO: 54 and a light chain variable region as set forth in SEQ ID NO: 77; in some preferred embodiments, the antibody or antigen-binding fragment thereof has a heavy chain variable region as set forth in SEQ ID NO: 54 and a light chain variable region as set forth in SEQ ID NO: 78; in some preferred embodiments, the antibody or antigen-binding fragment thereof has a heavy chain variable region as set forth in SEQ ID NO: 54 and a light chain variable region as set forth in SEQ ID NO: 79; in some preferred embodiments, the antibody or antigen-binding fragment thereof has a heavy chain variable region as set forth in SEQ ID NO: 54 and a light chain variable region as set forth in SEQ ID NO: 80. In some preferred embodiments, the antibody or antigen-binding fragment thereof has a heavy chain variable region as set forth in SEQ ID NO: 55 and a light chain variable region as set forth in SEQ ID NO: 77; in some preferred embodiments, the antibody or antigen-binding fragment thereof has a heavy chain variable region as set forth in SEQ ID NO: 55 and a light chain variable region as set forth in SEQ ID NO: 78; in some preferred embodiments, the antibody or antigen-binding fragment thereof has a heavy chain variable region as set forth in SEQ ID NO: 55 and a light chain variable region as set forth in SEQ ID NO: 79; in some preferred embodiments, the antibody or antigen-binding fragment thereof has a heavy chain variable region as set forth in SEQ ID NO: 55 and a light chain variable region as set forth in SEQ ID NO: 80. In some preferred embodiments, the antibody or antigen-binding fragment thereof has a heavy chain variable region as set forth in SEQ ID NO: 56 and a light chain variable region as set forth in SEQ ID NO: 77; in some preferred embodiments, the antibody or antigen-binding fragment thereof has a heavy chain variable region as set forth in SEQ ID NO: 56 and a light chain variable region as set forth in SEQ ID NO: 78; in some preferred embodiments, the antibody or antigen-binding fragment thereof has a heavy chain variable region as set forth in SEQ ID NO: 56 and a light chain variable region as set forth in SEQ ID NO: 79; in some preferred embodiments, the antibody or antigen-binding fragment thereof has a heavy chain variable region as set forth in SEQ ID NO: 56 and a light chain variable region as set forth in SEQ ID NO: 80. In some preferred embodiments, the antibody or antigen-binding fragment thereof has a heavy chain variable region as set forth in SEQ ID NO: 57 and a light chain variable region as set forth in SEQ ID NO: 77; in some preferred embodiments, the antibody or antigen-binding fragment thereof has a heavy chain variable region as set forth in SEQ ID NO: 57 and a light chain variable region as set forth in SEQ ID NO: 78; in some preferred embodiments, the antibody or antigen-binding fragment thereof has a heavy chain variable region as set forth in SEQ ID NO: 57 and a light chain variable region as set forth in SEQ ID NO: 79; in some preferred embodiments, the antibody or antigen-binding fragment thereof has a heavy chain variable region as set forth in SEQ ID NO: 57 and a light chain variable region as set forth in SEQ ID NO: 80.

In some preferred embodiments, the antibody or antigen-binding fragment thereof has a heavy chain variable region as set forth in SEQ ID NO: 85 and a light chain variable region as set forth in SEQ ID NO: 82. In some preferred embodiments, the antibody or antigen-binding fragment thereof has a heavy chain variable region as set forth in SEQ ID NO: 89 and a light chain variable region as shown in SEQ ID NO: 81.

In some embodiments, the antibodies or antigen-binding fragments thereof in the present disclosure (i) significantly activate total human T cells, upregulate CD25 expression and/or induce the release of IFN-γ; and/or (ii) bind to human CD3 (e.g. CD3e, in particular CD3e/d and/or CD3e/g) with a K_{D} value of less than 4×10⁻⁸ (e.g., less than 1×10⁻⁸, less than 1×10⁻⁹, less than 1×10⁻¹⁰ or less than 1×10⁻¹¹) M.

In addition, antibodies which compete for or cross-block the binding of the antibodies or antigen-binding fragments thereof disclosed herein to CD3, or which themselves are cross-blocked from binding to CD3 by the antibodies disclosed herein, may also be used in the present invention. In some cases, these competing, cross-blocking, or cross-blocked antibodies have an epitope which crosses and/or overlaps with the epitopes of the antibodies or antigen-binding fragments thereof disclosed herein. In some cases, these competing, cross-blocking, or cross-blocked antibodies have the same epitopes as the epitopes of the antibodies or antigen-binding fragments thereof disclosed herein.

Competing, cross-blocking, and cross-blocked antibodies can be identified using any suitable method known in the art, including competition ELISAs or BIACORE® assays where binding of the competing or cross-blocking antibody to human CD3 prevents the binding of an antibody disclosed herein or vice versa.

In some embodiments, the competing or cross-blocked antibodies are competed with or cross-blocked to greater than 80%, greater than 85%, greater than 90%, or greater than 95%. In some embodiments, the competing, cross-blocking, or cross-blocked antibodies are chimeric, fully human, or are humanized.

In addition, the antibodies or antigen-binding fragments thereof disclosed herein can also be used to construct a chimeric antigen receptor or a genetically modified cell, wherein the chimeric antigen receptor comprises the antibodies or antigen-binding fragments thereof disclosed herein as described above; the genetically modified cell is preferably an eukaryotic cell, more preferably an isolated human cell, and even more preferably an immune cell such as a T cell, or a NK cell such as a NK92 cell line.

The term “chimeric antigen receptor” or “CAR” refers to a polypeptide comprising an extracellular domain (extracellular binding domain) capable of binding an antigen, a hinge domain, a transmembrane domain (transmembrane region), and a peptide which enables the transmission of cytoplasmic signal to domain (i.e., an intracellular signaling domain). The hinge domain may be considered as a portion which provides flexibility to the extracellular antigen binding region. The intracellular signaling domain refers to a protein that transmits information into the cell to regulate cell activity by generating a second messenger via a certain signaling pathway, or a protein that acts as an effector corresponding to such messenger to generate signals that can promote immune effector function of CAR cells (e.g., CART cells). The intracellular signaling domain comprises a signaling domain and may also comprise a co-stimulatory intracellular domain derived from a co-stimulatory molecule.

The term “immune cell” refers to a cell that can trigger immune response, and “immune cell” and other grammatical forms thereof can refer to an immune cell of any origin. “Immune cell” includes, for example, white blood cells (leukocytes) derived from hematopoietic stem cells (HSCs) produced in bone marrow, lymphocytes (T cells, B cells, and natural killer (NK) cells), and cells of bone marrow origin (neutrophils, eosinophils, basophils, monocytes, macrophages, and dendritic cells). The term “immune cell” can also be human or non-human. For example, the immune cells can be derived from blood, such as autologous T cells, allogeneic T cells, autologous NK cells and allogeneic NK cells, or from cell lines, such as NK cell lines prepared using EBV virus infection, NK cells and NK92 cell lines induced and differentiated from embryonic stem cells and iPSCs, etc.

The antibodies or antigen-binding fragments thereof disclosed herein can also be used to construct an antibody-drug conjugate comprising a cytotoxic agent and an antibody or antigen-binding fragment thereof in the present disclosure as described above. In some embodiments, the antibodies or antigen-binding fragments described herein can be conjugated to a therapeutic agent, and the antibody-drug conjugate comprising the antibody or antigen-binding fragment thereof can be covalently or non-covalently bound to the therapeutic agent. In some embodiments, the therapeutic agent is a cytotoxic agent or a cell growth inhibitor (e.g., cytochalasin B, gramicidin D, ethidium bromide, emetine, mitomycin, etoposide, tenotoposide, vincristine, vinblastine, colchicine, doxorubicin, daunorubicin, dihydroxyanthraquinone, maytansinoid alkaloids (such as DM-1 and DM-4), diketones, serine, mitomycin D, 1-dehydrotestosterone, glucocorticoids, procaine, tetracaine, lidocaine, propranolol, puromycin, epirubicin and cyclophosphamide and analogs thereof).

### Pharmaceutical Composition

The present disclosure provides pharmaceutical compositions comprising antibodies or antigen-binding fragments thereof in the present disclosure. These pharmaceutical compositions comprise a therapeutically effective amount of the antibody or antigen-binding fragment thereof and one or more additional components, such as a physiologically acceptable carrier, excipient or diluent. In some embodiments, these additional components may include buffer(s), carbohydrate(s), polyol(s), amino acid(s), chelating agent(s), stabilizer(s), and/or preservative(s), etc.

In addition, the pharmaceutical composition may include a therapeutically effective amount of the antibody or antigen-binding fragment thereof and one or more additional therapeutic agents as active ingredients. For combination therapy, the antibody or antigen-binding fragment thereof as described above, the antibody-drug conjugate as described above, etc. and/or additional therapeutic or diagnostic agents can each be used as a single agent, and administered over any time range suitable for performing the intended treatment or diagnosis. Therefore, these single agents can be administered substantially simultaneously (i.e., either as a single formulation or within minutes or hours) or sequentially in succession.

In some embodiments, the additional therapeutic agents can comprise one or more inhibitors selected from the group consisting of a B-Raf inhibitor, an EGFR inhibitor, a MEK inhibitor, an ERK inhibitor, a K-Ras inhibitor, a c-Met inhibitor, an anaplastic lymphoma kinase (ALK) inhibitor, a phosphatidylinositol 3-kinase (PI3K) inhibitor, an Akt inhibitor, a mTOR inhibitor, a dual PI3K/mTOR inhibitor, a Bruton's tyrosine kinase (BTK) inhibitor, and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1 (IDO1) (e.g., epacadostat).

In some embodiments, the additional therapeutic agents can comprise one or more inhibitors selected from the group consisting of an HER3 inhibitor, an LSD 1 inhibitor, a MDM2 inhibitor, a BCL2 inhibitor, a CHK1 inhibitor, an inhibitor of activated hedgehog signaling pathway, and an agent that selectively degrades the estrogen receptor.

In some embodiments, the additional therapeutic agents can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agents can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a therapeutic agent targeting CXCL9, a therapeutic agent targeting CXCL10, a therapeutic agent targeting CCL5, an LFA-1 agonist, an ICAM1 agonist, and a selectin agonist.

The pharmaceutical composition can be administered according to known methods. The administration route is, for example, oral, through injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the composition can be administered by bolus injection or continuously by infusion, or by implantation device

### Preparation Method

The antibodies or antigen-binding fragments thereof in the present disclosure can be prepared by methods known in the art for antibody preparation (e.g., by chemical synthesis or by recombinant expression technology).

Therapeutic antibodies such as monoclonal antibodies can be developed by a variety of technologies and approaches, including hybridoma technology, phage display technology, single lymphocyte gene cloning technology, etc. The preparation of monoclonal antibodies by hybridoma technology is still the mainstream method for preparing therapeutic monoclonal antibodies.

The traditional hybridoma preparation technology was established by Kohler and Milstein 40 years ago (Kohler and Milstein 1975, Nature 256:495), and has now been widely used in the preparation and production of many related monoclonal antibodies in scientific research, diagnosis, and treatment. Although its basic method is still in use today, there have been changes, improvements and innovations in many aspects, including the use of different strains of animals such as transgenic animals, the introduction of electrofusion technology, and the application of high-efficiency screening technology equipment such as ClonePix equipment, making the application of hybridoma technology more diverse and more efficient. Monoclonal antibodies prepared from conventional animals such as mice can be cloned by conventional molecular biology methods to clone the antibody heavy chain variable region and light chain variable region genes, and the variable region genes can be grafted to human antibody constant region genes to form human-mouse chimeric antibodies (U.S. Pat. No. 4,816,567 to Cabilly et al.), to greatly reduce the immunogenicity when used in humans. Furthermore, the CDR domains of the variable regions of the mouse antibodies can be grafted onto the framework of the human antibodies, thereby reducing the composition of the mouse antibodies to less than 5%, greatly increasing the safety of the antibodies used in human body. Antibodies obtained through this approach are called humanized antibodies and are the main products in the antibody drug market at present (U.S. Pat. No. 5,225,539 to Winter, and U.S. Pat. Nos. 5,530,101, 5,585,089, 5,693,762 and 6,180,370 to Queen et al.).

The recombinant expression of an antibody requires the construction of an expression vector comprising a nucleic acid encoding the antibody. The preparation method of the nucleic acid is a conventional preparation method in the art, for example, it may include the following steps: obtaining a nucleic acid molecule encoding the antibody as described above by gene cloning technology, or obtaining a nucleic acid molecule encoding the antibody as described above by artificial full sequence synthesis.

Once the nucleic acid encoding the antibody has been obtained, a vector for producing the antibody can be produced by recombinant DNA technology. The vector can be obtained by conventional methods in the art, for example, be constructed by linking the nucleic acid molecule described in the present application to a suitable expression vector. The expression vector is any conventional vector in the art, as long as it can carry the aforementioned nucleic acid molecule. The vector includes a eukaryotic cell expression vector and/or a prokaryotic cell expression vector.

The expression vector is transferred to a host cell by conventional techniques, and the transfected cells are then cultured by conventional techniques to produce the antibodies of the present disclosure. The host cell is any conventional host cell in the art, as long as the above-mentioned recombinant expression vector can be stably replicated and the nucleic acid carried can be effectively expressed. The host cell can be a prokaryotic cell (e.g., an E. coli cell) and/or a eukaryotic cell (e.g., a HEK293 cell or a CHO cell).

### Use of the Present Invention

The antibodies or antigen-binding fragments thereof, isolated nucleic acids, nucleic acid vectors, host cells, antibody-drug conjugates and pharmaceutical compositions disclosed herein can be used to treat a variety of conditions, including, for example, various forms of cancer, infection, autoimmune or inflammatory conditions and/or fibrotic conditions. The antibodies or antigen-binding fragments thereof, isolated nucleic acids, nucleic acid vectors, host cells, antibody-drug conjugates and pharmaceutical compositions disclosed herein can also be used to suppress immune responses or activate T cells,

In some embodiments, the use of the antibodies or antigen-binding fragments thereof, isolated nucleic acids, nucleic acid vectors, host cells, antibody-drug conjugates and pharmaceutical compositions disclosed herein in the preparation of medicaments or kits for suppressing immune responses or activating T cells is provided. In some embodiments, the use of the antibodies or antigen-binding fragments thereof, isolated nucleic acids, nucleic acid vectors, host cells, antibody-drug conjugates and pharmaceutical compositions disclosed herein in the preparation of medicaments or kits for preventing, treating or alleviating diseases is provided. In some embodiments, the disease is cancer or an autoimmune disease.

In some embodiments, the cancer includes but is not limited to: melanoma, renal cancer, prostate cancer, breast cancer, colorectal cancer, lung cancer, nasopharyngeal cancer, oral cancer, liver cancer, bone cancer, pancreatic cancer, skin cancer, head and neck cancer, uterine cancer, ovarian cancer, rectal cancer, stomach cancer, testicular cancer, fallopian tube cancer, endometrial cancer, cervical cancer, vaginal cancer, vulvar cancer, Hodgkin's lymphoma, non-Hodgkin's lymphoma, esophageal cancer, small intestine cancer, large intestine cancer, endocrine system cancer, thyroid cancer, parathyroid cancer, adrenal cancer, soft tissue sarcoma, urethral cancer, penile cancer, chronic or acute leukemia, bladder cancer, renal pelvis cancer, central nervous system tumor, glioma, spinal axis tumor, brain stem glioma, pituitary adenoma, Kaposi's sarcoma, epidermoid carcinoma, squamous cell carcinoma, or T-cell lymphoma.

In some embodiments, the autoimmune disease includes, but is not limited to, rheumatoid arthritis, multiple sclerosis, Sjögren's syndrome, insulin-dependent diabetes mellitus, autoimmune thyroiditis, reactive arthritis, ankylosing spondylitis, scleroderma, polymyositis, dermatomyositis, psoriasis, Wegener's granulomatosis, Crohn's disease, ulcerative colitis, lupus such as systemic lupus erythematosus, atherosclerosis, chronic obstructive pulmonary disease, cirrhosis, renal transplant fibrosis, renal transplant nephropathy, or pulmonary fibrosis.

The present invention is further illustrated below by way of examples, but is not limited to the scope of the examples. If the experimental methods are not specified with specific conditions in the following examples, conventional methods and conditions are used, or selected according to the product specifications.

### EXAMPLE

### Example 1. Preparation of CD3 Antibodies

### (i). Preparation of Immunogens

CD3e/g is a soluble ligand, and we designed the products of CD3e/g proteins and human constant regions—CD3e/g recombinant proteins as immunogens. The specific sequences of CD3e/g proteins are shown in Table 1.

TABLE 1
Immunogen sequences

| No. | sequence |
|---|---|
| Human | MQSGTHWRVLGLCLLSVGVWGQDGNEEMGGITQTPYK |
| CD3e/g | VSISGTTVILTCPQYPGSEILWQHNDKNIGGDEDDKN |
| protein | IGSDEDHLSLKEFSELEQSGYYVCYPRGSKPEDANFY |
| | LYLRARVCENCMEMDVMSVATIVIVDICITGGLLLLV |
| | YYWSKNRKAKAKPVTRGAGAGGRQRGQNKERPPPVPN |
| | PDYEPIRKGQRDLYSGLNQRRIMEQGKGLAVLILAII |
| | LLQGTLAQSIKGNHLVKVYDYQEDGSVLLTCDAEAKN |
| | ITWFKDGKMIGFLTEDKKKWNLGSNAKDPRGMYQCKG |
| | SQNKSKPLQVYYRMCQNCIELNAATISGFLFAEIVSI |
| | FVLAVGVYFIAGQDGVRQSRASDKQTLLPNDQLYQPL |
| | KDREDDQYSHLQGNQLRRN |
| | (SEQ ID NO: 83) |
| Cynomolgus | DGNEEMGSITQTPYQVSISGTTVILTCSQHLGSEAQW |
| monkey | QHNGKNKEDSGDRLFLPEFSEMEQSGYYVCYPRGSNP |
| CD3e/g | EDASHHLYLKARVCENCMEMDGSSGSSDKTHTCPPCP |
| protein | APELLGGPSVFLFPPKPKDTLMISRTPEVTCVVVDVS |
| | HEDPEVKFNWYVDGVEVHNAKTKPREEQYNSTYRVVS |
| | VLTVLHQDWLNGKEYKCKVSNKALPAPIEKTISKAKG |
| | QPREPQVYTLPPCRDELTKNQVSLWCLVKGFYPSDIA |
| | VEWESNGQPENNYKTTPPVLDSDGSFFLYSKLTVDKS |
| | RWQQGNVFSCSVMHEALHNHYTQKSLSLSPGKQSFEE |
| | NRKLNVYNQEDGSVLLTCHVKNTNITWFKEGKMIDIL |
| | TAHKNKWNLGSNTKDPRGVYQCKGSKDKSKTLQVYYR |
| | MCQNGSSGSSDKTHTCPPCPAPELLGGPSVFLFPPKP |
| | KDTLMISRTPEVTCVVVDVSHEDPEVKFNWYVDGVEV |
| | HNAKTKPREEQYNSTYRVVSVLTVLHQDWLNGKEYKC |
| | KVSNKALPAPIEKTISKAKGQPREPQVCTLPPSRDEL |
| | TKNQVSLSCAVKGFYPSDIAVEWESNGQPENNYKTTP |
| | PVLDSDGSFFLVSKLTVDKSRWQQGNVFSCSVMHEAL |
| | HNHYTQKSLSLSPGK |
| | (SEQ ID NO: 84) |

### (ii). Preparation of Hybridoma Cells and Antibody Screening

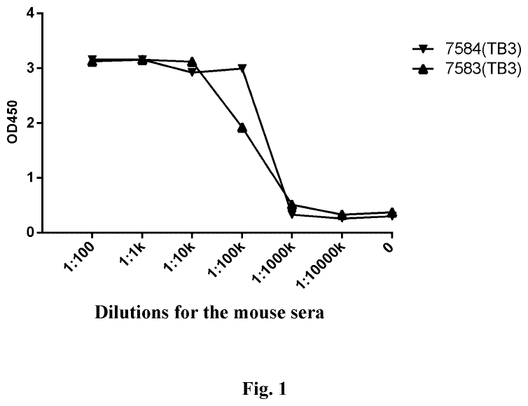

The above-mentioned immunogens were used to immunize 6-8 week old SJL mice (purchased from Shanghai SLAC Co, Ltd.), and the mice were raised under SPF conditions. During the first immunization, the immunogens were emulsified with Freund's complete adjuvant and then injected into the tail vein end with 0.25 ml, that is, each mouse was injected with 50 micrograms of immunogen. During the booster immunization, the immunogens were emulsified with Freund's incomplete adjuvant and then injected into the tail vein end with 0.25 ml, that is, each mouse was injected with 50 micrograms of immunogen. There was a 2-week interval between the first immunization and the first booster immunization, and then a 3-week interval between each booster immunization. Blood was collected 1 week after each booster immunization, and the antibody titers and specificities for the immunogens in the serum were detected by ELISA. The results are shown in FIG. 1 and Table 2. As can be seen from FIG. 1 and Table 2, the mouse sera immunized with CD3e/g recombinant proteins have different degrees of binding to the immunogens, showing antigen-antibody reaction, with the highest dilution being around one thousand. The blank control was 1% (w/w) BSA, the batch referred to the mouse serum on the seventh day after the second booster immunization, and the data in the table are OD₄₅₀ nm values.

TABLE 2
Antibody titers of mouse sera after immunization with
CD3e/g recombinant proteins as detected by ELISA

| OD_{450 nm} | Serum dilution | | | | | | |
|---|---|---|---|---|---|---|---|
| Batch | 1:10² | 1:10³ | 1:10⁴ | 1:10⁵ | 1:10⁶ | 1:10⁷ | Blank control |
| 7583(TB3) | 3.13 | 3.16 | 3.12 | 1.92 | 0.51 | 0.33 | 0.37 |
| 7584(TB3) | 3.16 | 3.16 | 2.92 | 3.00 | 0.33 | 0.26 | 0.30 |

Before the immunization step was completed, each selected mouse was intraperitoneally injected with 100 micrograms of CD3e/g recombinant protein for the last immunization. The mice were killed 3 days later and spleen cells were collected. NH₄OH was added to a final concentration of 1% (w/w) to lyse the red blood cells mixed in the spleen cells to obtain a spleen cell suspension. The cells were washed three times by centrifugation at 1000 rpm with DMEM basal medium, and then mixed with mouse myeloma cells SP2/0 (purchased from ATCC #CRL-1581) at a ratio of 5:1 in terms of the number of live cells, and cell fusion was performed using a high-efficiency electrofusion method (see METHODS IN ENZYMOLOGY, VOL. 220). The cells after fusion were diluted into DMEM medium containing 20% fetal bovine serum and 1×HAT, wherein the percentage was a mass percentage. Then, 1×10⁵/200 microliter per well were added to a 96-well cell culture plate and placed in a 5% CO₂ and 37° C. incubator, wherein the percentage was a volume percentage. After 14 days, the supernatant of the cell fusion plate was screened with an ELISA plate coated with the antigen CD3e/g, and the positive clones with an OD value greater than 2 in the ELISA results were expanded to a 24-well plate and amplificatorily cultured in DMEM (purchased from Invitrogen, catalog number 11995-065) containing 10% (w/w) HT fetal bovine serum at 37° C. and 5% (v/v) CO₂. After 3 days of culture, the culture fluid of the amplification culture in the 24-well plate was centrifuged, the supernatant was collected, the antibody subtype analysis of the supernatant was performed, and the binding activity to the antigen was determined by ELISA.

According to the results of 24-well plate screening, hybridoma cells with OD_{450 nm}>2 in ELISA detection were selected as qualified positive clones. The qualified hybridoma cells were selected to be subcloned in 96-well plates by limiting dilution method and cultured in DMEM medium (purchased from Invitrogen) containing 10% (w/w) FBS at 37° C. and 5% (v/v) CO₂. 10 days after subcloning, ELISA was used for preliminary screening, and a single positive monoclonal clone was selected to be expanded to a 24-well plate for further culture. 3 days later, ELISA was used to determine positive for antigen binding and the CD3e/g receptor ligand binding experiment was used to evaluate biological activity (the evaluation standard was OD_{450 nm} value>2 in the ELISA experiment).

Based on the test results of samples from the 24-well plate, clones were selected and amplificatorily cultured in DMEM medium (purchased from Invitrogen) containing 10% (w/w) FBS at 37° C. and 5% (v/v) CO₂. The hybridoma cells of the present invention were obtained by freezing in liquid nitrogen and could be used for subsequent antibody production and purification.

### Example 2. Production and Purification of CD3 Antibodies

The hybridoma cells obtained in Example 1 were inoculated into T-75 cell culture flasks and acclimatized and subcultured for 3 generations using production medium (Hybridoma serum free medium, purchased from Invitrogen). When the cells are in good growth state, they were inoculated into cell culture roller bottles. 500 ml of production medium was added to each 2-liter culture roller bottle, and the inoculation cell density was 1.0×10⁵/ml. The culture roller bottles were placed on a rotary machine at a speed of 3 rpm in a 37° C. incubator. After continuous rotation culture for 14 days, the cell culture fluids were collected, filtered to remove cells, and filtered with a 0.45 μm filter membrane until the culture supernatants were clarified. The clarified culture supernatants could be purified immediately or frozen at −30° C.

The antibodies in the clarified hybridoma cell culture supernatants (300 mL) were purified using a 2 mL protein A column (purchased from GE Healthcare). The protein A column was first equilibrated with an equilibration buffer (PBS phosphate buffer, pH 7.2), and then the clarified culture supernatants were loaded onto the protein A column, with the flow rate controlled at 3 mL/min. After loading, the protein A column was washed with the equilibration buffer at a volume 4 times the volume of the protein A column bed. The CD3e/g antibodies bound to the protein A column were eluted with an eluent (0.1 M glycine hydrochloride buffer, pH 2.5), and the elution was monitored with a UV detector (A280 UV absorption peak). The eluted antibodies were collected, added with 10% 1.0 M Tris-HCl buffer to neutralize the pH, wherein the percentage was a volume percentage, and then immediately dialyzed with PBS phosphate buffer overnight. The fluid exchange was performed once the next day and the dialysis continued for 3 hours. The dialyzed CD3e/g antibodies were collected, aseptically filtered using a 0.22 μm filter, and aseptically stored to obtain the purified CD3e/g antibodies.

### Example 3. Antigen Binding Activity of CD3 Antibodies

Enzyme-linked immunosorbent assay (ELISA) was used to detect antigen-antibody binding sites.

The purified CD3e/g antibodies obtained in Example 2 were subjected to binding reaction with CD3e/g (sequences shown in Table 1) and CD3e/d proteins (purchased from Sinobiological, CAT: CT038-H2508H-B).

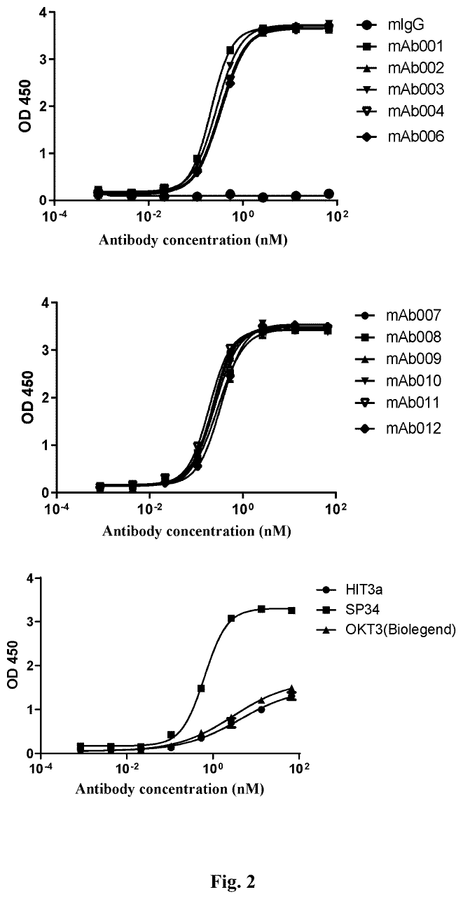

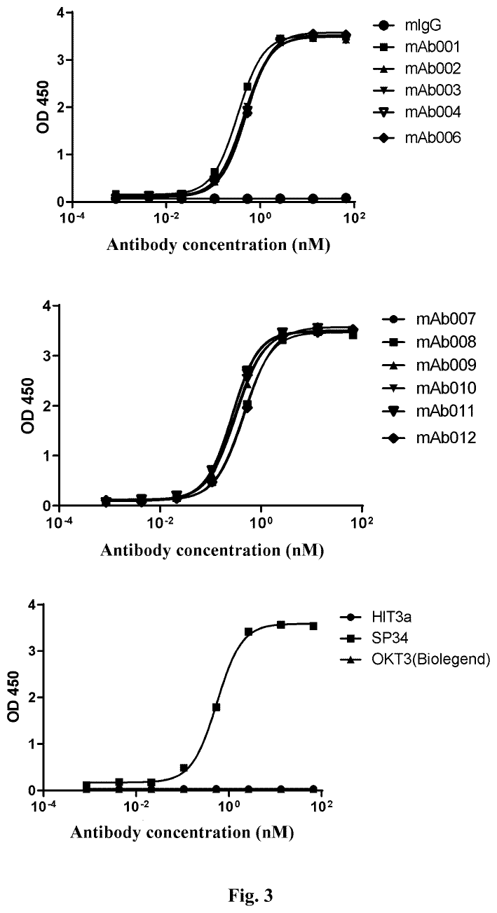

Human CD3e/g protein, cynomolgus monkey CD3e/g protein (sequences shown in Table 1) or human CD3e/d protein was diluted with PBS to a final concentration of 5.0 μg/mL, and then added with 100 μl per well to a 96-well ELISA plate. The plate was sealed with plastic film and incubated overnight at 4° C. The plate was washed twice with plate washing solution [PBS+0.01% (v/v) Tween 20] on the next day, and was added with blocking solution [PBS+0.01% (v/v) Tween 20+1% (w/w) BSA] to block at room temperature for 2 hours. The blocking solution was poured off, and 100 μl per well of the purified CD3e/g antibody obtained in Example 2 was added. After incubation at 37° C. for 2 hours, the plate was washed 3 times with plate washing solution [PBS+0.01% (v/v) Tween20]. After adding HRP (horseradish peroxidase) labeled secondary antibody (purchased from Sigma), the plate was incubated at 37° C. for 2 hours, washed 3 times with plate washing solution [PBS+0.01% (v/v) Tween 20]. After adding 100 μl per well of TMB substrate and incubating at room temperature for 30 minutes, 100 μl per well of stop solution (1.0N HCl) was added. The A450 nm value was read using an ELISA plate reader (SpectraMax 384plus, purchased from Molecular Device). FIG. 2 and Table 3 show the binding activities of CD3e/g antibodies to hCD3e/g as detected by ELISA, and FIG. 3 and Table 4 show the binding activities of CD3e/g antibodies to cyno CD3e/g as detected by ELISA, wherein the IgG control is mouse IgG, and the data in the tables are OD_{450 nm} values. Among them, HIT3a was purchased from BD pharmigen with a catalog number 555336; SP34 was purchased from BD pharmigen with a catalog number 551916. OKT3 was purchased from Biolegend.

As can be seen from FIGS. 2 and 3, and Tables 3 and 4, the antibodies of the present disclosure can bind to human CD3e/g (hCD3e/g), and the binding affinity of the antibodies of the present disclosure to human CD3e/g is better than that of OKT3. Also, the antibodies of the present disclosure can bind to cynomolgus monkey CD3 (cyno CD3e/g), while OKT3 has no binding affinity or no significant binding to cynomolgus monkey CD3e/g.

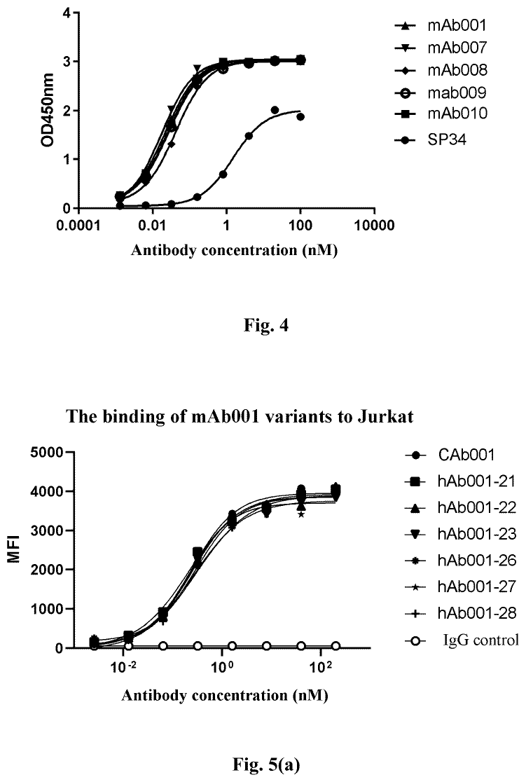

As can be seen from FIG. 4 and Table 5, the antibodies of the present disclosure can also bind to human CD3e/d (hCD3e/d), and the binding affinity of the antibodies of the present disclosure to human CD3e/d is better than that of SP34.

TABLE 3
Binding of CD3e/g antibodies to hCD3e/g as detected by ELISA

| | | | | | | | | | EC₅₀ |
|---|---|---|---|---|---|---|---|---|---|
| OD_{450 nm} | hCD3e/g concentration (nM) | | | | | | | | (nM) |
| Antibody No. | 66.7 | 13.3 | 2.67 | 0.53 | 0.11 | 0.02 | 0.0043 | 0.000853 | |
| mIgG | 0.15 | 0.1 | 0.07 | 0.14 | 0.09 | 0.08 | 0.1012 | 0.17735 | |
| OKT3 | 1.53 | 1.21 | 0.80 | 0.47 | 0.17 | 0.09 | 0.08 | 0.07 | 2.95 |
| HIT3a | 1.22 | 0.94 | 0.59 | 0.31 | 0.12 | 0.08 | 0.07 | 0.07 | 4.2 |
| SP34 | 3.23 | 3.29 | 3.10 | 1.45 | 0.38 | 0.15 | 0.10 | 0.18 | 0.64 |
| mAb001 | 3.67 | 3.67 | 3.64 | 3.2 | 0.89 | 0.24 | 0.1199 | 0.20665 | 0.21 |
| mAb002 | 3.63 | 3.64 | 3.57 | 2.57 | 0.65 | 0.19 | 0.1172 | 0.1243 | 0.32 |
| mAb003 | 3.76 | 3.69 | 3.66 | 2.86 | 0.77 | 0.21 | 0.1535 | 0.23055 | 0.28 |
| mAb004 | 3.65 | 3.62 | 3.59 | 2.57 | 0.59 | 0.25 | 0.1414 | 0.1013 | 0.33 |
| mAb006 | 3.73 | 3.69 | 3.58 | 2.49 | 0.62 | 0.26 | 0.1399 | 0.11275 | 0.35 |
| mAb007 | 3.41 | 3.47 | 3.39 | 2.96 | 0.76 | 0.27 | 0.1108 | 0.123 | 0.22 |
| mAb008 | 3.42 | 3.43 | 3.39 | 2.51 | 0.72 | 0.29 | 0.1491 | 0.1236 | 0.29 |
| mAb009 | 3.42 | 3.52 | 3.49 | 2.87 | 0.84 | 0.28 | 0.1075 | 0.10445 | 0.23 |
| mAb010 | 3.38 | 3.47 | 3.56 | 2.74 | 0.8 | 0.29 | 0.1298 | 0.14335 | 0.25 |
| mAb011 | 3.45 | 3.42 | 3.36 | 3.03 | 0.97 | 0.31 | 0.1591 | 0.1098 | 0.19 |
| mAb012 | 3.5 | 3.53 | 3.51 | 2.46 | 0.56 | 0.21 | 0.1682 | 0.12305 | 0.34 |

TABLE 4
Binding of CD3e/g antibodies to cyno CD3e/g as detected by ELISA

| | | | | | | | | | EC₅₀ |
|---|---|---|---|---|---|---|---|---|---|
| OD_{450 nm} | cyno CD3e/g concentration (nM) | | | | | | | | (nM) |
| Antibody No. | 66.7 | 13.3 | 2.67 | 0.53 | 0.11 | 0.02 | 0.0043 | 0.000853 | |
| mIgG | 0.08 | 0.07 | 0.07 | 0.06 | 0.07 | 0.07 | 0.1008 | 0.0698 | |
| OKT3 | 0.26 | 0.25 | 0.03 | 0.04 | 0.04 | 0.04 | 0.04 | 0.04 | — |
| HIT3a | 0.04 | 0.04 | 0.04 | 0.04 | 0.04 | 0.04 | 0.04 | 0.04 | — |
| SP34 | 3.55 | 3.57 | 3.41 | 1.83 | 0.50 | 0.19 | 0.20 | 0.12 | 0.55 |
| mAb001 | 3.49 | 3.5 | 3.45 | 2.43 | 0.64 | 0.17 | 0.1519 | 0.1637 | 0.33 |
| mAb002 | 3.43 | 3.47 | 3.37 | 1.89 | 0.43 | 0.14 | 0.0919 | 0.0647 | 0.49 |
| mAb003 | 3.45 | 3.48 | 3.39 | 2.01 | 0.5 | 0.16 | 0.1045 | 0.0895 | 0.44 |
| mAb004 | 3.45 | 3.48 | 3.38 | 1.92 | 0.46 | 0.14 | 0.1259 | 0.06555 | 0.48 |
| mAb006 | 3.53 | 3.55 | 3.43 | 1.88 | 0.45 | 0.13 | 0.1151 | 0.14165 | 0.51 |
| mAb007 | 3.49 | 3.47 | 3.45 | 2.7 | 0.73 | 0.22 | 0.1282 | 0.0902 | 0.26 |
| mAb008 | 3.42 | 3.48 | 3.32 | 2.03 | 0.48 | 0.16 | 0.1106 | 0.08935 | 0.44 |
| mAb009 | 3.46 | 3.54 | 3.41 | 2.44 | 0.63 | 0.19 | 0.1259 | 0.07545 | 0.32 |
| mAb010 | 3.46 | 3.5 | 3.43 | 2.5 | 0.67 | 0.18 | 0.0981 | 0.06915 | 0.30 |
| mAb011 | 3.45 | 3.47 | 3.46 | 2.65 | 0.7 | 0.2 | 0.1212 | 0.0668 | 0.27 |
| mAb012 | 3.53 | 3.56 | 3.4 | 1.96 | 0.47 | 0.14 | 0.074 | 0.10875 | 0.48 |

TABLE 5
Binding of CD3e/g antibodies to hCD3e/d as detected by ELISA

| | | | | | | | | | Eₘₐₓ | EC₅₀ |
|---|---|---|---|---|---|---|---|---|---|---|
| OD450 nm | hCD3e/d concentration (nM) | | | | | | | | (OD) | (nM) |
| Antibody No. | 100 | 20 | 4 | 0.8 | 0.16 | 0.032 | 0.0064 | 0.00128 | | |
| mAb001 | 3.02 | 3.05 | 2.99 | 2.96 | 2.72 | 1.83 | 0.72 | 0.27 | 3.050 | 0.022 |
| mAb007 | 3.04 | 3.01 | 2.99 | 2.96 | 2.86 | 2.02 | 0.71 | 0.26 | 3.040 | 0.017 |
| mAb008 | 3.05 | 3.00 | 2.96 | 2.88 | 2.48 | 1.31 | 0.53 | 0.16 | 3.052 | 0.041 |
| mAb009 | 3.03 | 3.02 | 2.97 | 2.85 | 2.64 | 1.67 | 0.66 | 0.24 | 3.034 | 0.027 |
| mAb010 | 3.05 | 3.02 | 3.00 | 3.00 | 2.65 | 1.71 | 0.69 | 0.25 | 3.050 | 0.026 |
| SP34 | 1.87 | 2.01 | 1.49 | 0.70 | 0.23 | 0.09 | 0.06 | 0.05 | 2.012 | 1.506 |

### Example 4. Humanization Engineering of Antibodies

The heavy chain and light chain variable region (V_{H} and V_{L}) sequences of mouse anti-human CD3 antibodies were used to retrieve the corresponding human antibody heavy and light chain variable region sequences in the IMGT database. Using MOE software, by aligning the gene sequences of the mouse antibody heavy chain and light chain variable regions and the gene sequences of the retrieved human antibody heavy and light chain variable regions, the heavy chain and light chain variable region germline genes with high homology to the mouse antibodies were selected as templates, and the CDRs of the mouse antibodies were grafted into the corresponding human templates to form variable region sequences in the order of FRI-CDR1-FR2-CDR2-FR3-CDR3-FR4. According to the structural analysis of the selected human antibody heavy chain variable regions and light chain variable regions against the mouse antibodies to maintain the affinity of the original mouse antibodies, the key amino acids in the framework sequences were back-mutated to the corresponding amino acids of the mouse antibodies, and the humanized anti-CD3 monoclonal antibodies were obtained. The amino acid residues in the CDR regions were determined and annotated by the Kabat numbering system.

The light and heavy chain variable regions of the above-mentioned mouse antibodies were linked to the light and heavy chain constant regions of the human antibodies to form chimeric antibodies. The chimeric antibody corresponding to the mAb001 antibody was named CAb001, and the same applies to other antibodies.

### 3.1 Humanization of Mouse Anti-mAb001

3.1.1 mAb001 Humanization Scheme 1
(1) Selection of Frameworks for Humanization of mAb001

For the mouse antibody mAb001, the light chain template for humanization was IGKV1-39 IGKV1-39*01 X59315 V-KAPPA, and the heavy chain template for humanization was IGHV1-8 IGHV1-8*01 M99637 VH. After humanization, the humanized antibody hAb001 was obtained. The sequences of the humanized variable regions are as follows:

>hAb001 VH1-CDR grafted
(SEQ ID NO: 33)
QVQLVQSGAEVKKPGASVKVSCKASGYSFTNYYMHWVRQATGQGLEWMGW
TYPGNNNIKYNEKFKGRVTMTRNTSISTAYMELSSLRSEDTAVYYCARDG
YGYYFFDYWGQGTLVTVSS
>hAb001 VL1-CDR grafted
(SEQ ID NO: 58)
DIQMTQSPSSLSASVGDRVTITCKSSQSLLNNRTRKNYLAWYQQKPGKAP
KLLIYWASTRESGVPSRFSGSGSGTDFTLTISSLQPEDFATYYCKQSYTL
RTFGGGTKVEIK

Note: The order is FR1-CDR1-FR2-CDR2-FR3-CDR3-FR4; the underlined sequences are the CDR sequences, and the ununderlined sequences are the FR sequences.

(2) the Back Mutation Design for hAb001 is as Follows:

| | | Donor | | | Donor | |
|---|---|---|---|---|---|---|
| | | Framework | SEQ | | Framework | SEQ |
| Antibody | Light Chain | Residue and | ID | Heavy | Residue and | ID |
| Name | Name | Back Mutation | NO. | Chain Name | Back Mutation | NO. |
| hAb001 | mAb001VL_hum1 | grafted | 58 | mAb001VH_hum1 | grafted | 33 |
| | mAb001VL_hum2 | Q3V, N32Q, | 59 | mAb001VH_hum2 | N55Q, I76S | 34 |
| | mAb001VL_hum3 | I21M | 60 | mAb001VH_hum3 | N56Q, R67K, | 35 |
| | | | | | T91S, L114I | |
| | mAb001VL_hum4 | Q3V, I21M, | 61 | mAb001VH_hum4 | N55Q, N57Q, R67K, | 36 |
| | | N32Q, S69T | | | I76S, T91S, T113I | |

Note:
For example, Q3V means that the Q at position 3 was mutated back to V, numbering according to the natural order of the amino acid sequence. The “grafted” means CDRs of the mouse antibody were engrafted into the human germline FR region sequences.

(3) the Sequence Combinations for Humanization for hAb001 are as Follows:

| | mAb001VL_hum1 | mAb001VL_hum2 | mAb001VL_hum3 | mAb001VL_hum4 |
|---|---|---|---|---|
| mAb001VH_hum1 | hAb001-1 | hAb001-2 | hAb001-3 | hAb001-4 |
| mAb001VH_hum2 | hAb001-5 | hAb001-6 | hAb001-7 | hAb001-8 |
| mAb001VH_hum3 | hAb001-9 | hAb001-10 | hAb001-11 | hAb001-12 |
| mAb001VH_hum4 | hAb001-13 | hAb001-14 | hAb001-15 | hAb001-16 |

Note:
This table represents the sequences obtained by combining each mutations. For example, hAb001-6 means that there are four back mutations from light chain mAb001VL_hum2 and heavy chain mAb001VH_hum2 on the humanized antibody hAb001-6. The same applies to other sequences.

(4) the Specific Sequences for Humanization for hAb001 are as Follows:

>mAb001VL_hum1
(SEQ ID NO: 58)
DIQMTQSPSSLSASVGDRVTITCKSSQSLLNNRTRKNYLAWYQQKPGKAP
KLLIYWASTRESGVPSRFSGSGSGTDFTLTISSLQPEDFATYYCKQSYTL
RTFGGGTKVEIK
>mAb001VL_hum2
(SEQ ID NO: 59)
DIVMTQSPSSLSASVGDRVTITCKSSQSLLNQRTRKNYLAWYQQKPGKAP
KLLIYWASTRESGVPSRFSGSGSGTDFTLTISSLQPEDFATYYCKQSYTL
RTFGGGTKVEIK
>mAb001VL_hum3
(SEQ ID NO: 60)
DIQMTQSPSSLSASVGDRVTMTCKSSQSLLNNRTRKNYLAWYQQKPGKAP
KLLIYWASTRESGVPSRFSGSGSGTDFTLTISSLQPEDFATYYCKQSYTL
RTFGGGTKVEIK
>mAb001VL_hum4
(SEQ ID NO: 61)
DIVMTQSPSSLSASVGDRVTMTCKSSQSLLNQRTRKNYLAWYQQKPGKAP
KLLIYWASTRESGVPSRFTGSGSGTDFTLTISSLQPEDFATYYCKQSYTL
RTFGGGTKVEIK
>mAb001VH_hum1
(SEQ ID NO: 33)
QVQLVQSGAEVKKPGASVKVSCKASGYSFTNYYMHWVRQATGQGLEWMGW
TYPGNNNIKYNEKFKGRVTMTRNTSISTAYMELSSLRSEDTAVYYCARDG
YGYYFFDYWGQGTLVTVSS
>mAb001VH_hum2
(SEQ ID NO: 34)
QVQLVQSGAEVKKPGASVKVSCKASGYSFTNYYMHWVRQATGQGLEWMGW
TYPGQNNIKYNEKFKGRVTMTRNTSSSTAYMELSSLRSEDTAVYYCARDG
YGYYFFDYWGQGTLVTVSS
>mAb001VH_hum3
(SEQ ID NO: 35)
QVQLVQSGAEVKKPGASVKVSCKASGYSFTNYYMHWVRQATGQGLEWMGW
TYPGNQNIKYNEKFKGKVTMTRNTSISTAYMELSSLRSEDSAVYYCARDG
YGYYFFDYWGQGTIVTVSS
>mAb001VH_hum4
(SEQ ID NO: 36)
QVQLVQSGAEVKKPGASVKVSCKASGYSFTNYYMHWVRQATGQGLEWMGW
TYPGQNQIKYNEKFKGKVTMTRNTSSSTAYMELSSLRSEDSAVYYCARDG
YGYYFFDYWGQGILVTVSS

3.1.2 mAb001 Humanization Scheme 2
(1) Selection of Frameworks for Humanization of mAb001

For the mouse antibody mAb001, the light chain template for humanization was IGKV4-1 IGKV4-1*01 Z0023 V-KAPPA, and the heavy chain template for humanization was IGHV1-3 IGHV1-3*01 X62109 VH. After humanization, the humanized antibody hAb001 was obtained. The sequences of the humanized variable regions are as follows:

>hAb001 VH2-CDR grafted
(SEQ ID NO: 37)
QVQLVQSGAEVKKPGASVKVSCKASGYSFTNYYMHWVRQAPGQRLEWMGW
TYPGNNNIKYNEKFKGRVTITRDTSASTAYMELSSLRSEDTAVYYCARDG
YGYYFFDYWGQGTLVTVSS
>hAb001 VL2-CDR grafted
(SEQ ID NO: 62)
DIVMTQSPDSLAVSLGERATINCKSSQSLLNNRTRKNYLAWYQQKPGQPP
KLLIYWASTRESGVPDRFSGSGSGTDFTLTISSLQAEDVAVYYCKQSYTL
RTFGGGTKLEIK

Note: The order is FR1-CDR1-FR2-CDR2-FR3-CDR3-FR4; the underlined sequences are the CDR sequences, and the ununderlined sequences are the FR sequences.

(2) the Back Mutation Design for hAb001 is as Follows:

| | | Donor | | | Donor | |
|---|---|---|---|---|---|---|
| | | Framework | SEQ | | Framework | SEQ |
| Antibody | Light Chain | Residue and | ID | Heavy | Residue and | ID |
| Name | Name | Back Mutation | NO. | Chain Name | Back Mutation | NO. |
| hAb001 | mAb001VL_hum5 | grafted | 62 | mAb001VH_hum5 | grafted | 37 |
| | mAb001VL_hum6 | S69T | 63 | mAb001VH_hum6 | M48I, R72A | 38 |
| | mAb001VL_hum7 | I21M, N22S | 64 | mAb001VH_hum7 | M48I, V68A, | 39 |
| | | | | | R72A | |
| | mAb001VL_hum8 | S69T, I21M, | 65 | mAb001VH_hum8 | R38K, M48I, | 40 |
| | | N22S | | | V68A, R72A | |

Note:
For example, S69T means that the S at position 69 was mutated back to T, numbering according to the natural order of the amino acid sequence. The “grafted” means CDRs of the mouse antibody were engrafted into the human germline FR region sequences.

(3) The sequence combinations for humanization for hAb001 are as follows:

| | mAb001VL_hum5 | mAb001VL_hum6 | mAb001VL_hum7 | mAb001VL_hum8 |
|---|---|---|---|---|
| mAb001VH_hum5 | hAb001-17 | hAb001-18 | hAb001-19 | hAb001-20 |
| mAb001VH_hum6 | hAb001-21 | hAb001-22 | hAb001-23 | hAb001-24 |
| mAb001VH_hum7 | hAb001-25 | hAb001-26 | hAb001-27 | hAb001-28 |
| mAb001VH_hum8 | hAb001-29 | hAb001-30 | hAb001-31 | hAb001-32 |

Note:
This table represents the sequences obtained by combining each mutation. For example, hAb001-22 means that there are three back mutations from light chain mAb001VL_hum6 and heavy chain mAb001VH_hum6 on the humanized antibody hAb001-22. The same applies to other sequences.

(4) the Specific Sequences for Humanization for hAb001 are as Follows:

>mAb001VL_hum5
(SEQ ID NO: 62)
DIVMTQSPDSLAVSLGERATINCKSSQSLLNNRTRKNYLAWYQQKPGQPP
KLLIYWASTRESGVPDRFSGSGSGTDFTLTISSLQAEDVAVYYCKQSYTL
RTFGGGTKLEIK
>mAb001VL_hum6
(SEQ ID NO: 63)
DIVMTQSPDSLAVSLGERATINCKSSQSLLNNRTRKNYLAWYQQKPGQPP
KLLIYWASTRESGVPDRFTGSGSGTDFTLTISSLQAEDVAVYYCKQSYTL
RTFGGGTKLEIK
>mAb001VL_hum7
(SEQ ID NO: 64)
DIVMTQSPDSLAVSLGERATMSCKSSQSLLNNRTRKNYLAWYQQKPGQPP
KLLIYWASTRESGVPDRFSGSGSGTDFTLTISSLQAEDVAVYYCKQSYTL
RTFGGGTKLEIK
>mAb001VL_hum8
(SEQ ID NO: 65)
DIVMTQSPDSLAVSLGERATMSCKSSQSLLNNRTRKNYLAWYQQKPGQPP
KLLIYWASTRESGVPDRFTGSGSGTDFTLTISSLQAEDVAVYYCKQSYTL
RTFGGGTKLEIK
>mAb001VH_hum5
(SEQ ID NO: 37)
QVQLVQSGAEVKKPGASVKVSCKASGYSFTNYYMHWVRQAPGQRLEWMGW
TYPGNNNIKYNEKFKGRVTITRDTSASTAYMELSSLRSEDTAVYYCARDG
YGYYFFDYWGQGTLVTVSS
>mAb001VH_hum6
(SEQ ID NO: 38)
QVQLVQSGAEVKKPGASVKVSCKASGYSFTNYYMHWVRQAPGQRLEWIGW
TYPGNNNIKYNEKFKGRVTITADTSASTAYMELSSLRSEDTAVYYCARDG
YGYYFFDYWGQGTLVTVSS
>mAb001VH_hum7
(SEQ ID NO: 39)
QVQLVQSGAEVKKPGASVKVSCKASGYSFTNYYMHWVRQAPGQRLEWIGW
TYPGNNNIKYNEKFKGRATITADTSASTAYMELSSLRSEDTAVYYCARDG
YGYYFFDYWGQGTLVTVSS
>mAb001VH_hum8
(SEQ ID NO: 40)
QVQLVQSGAEVKKPGASVKVSCKASGYSFTNYYMHWVKQAPGQRLEWIGW
TYPGNNNIKYNEKFKGRATITADTSASTAYMELSSLRSEDTAVYYCARDG
YGYYFFDYWGQGTLVTVSS

### 3.2 Humanization of Mouse Anti-mAb007

### 3.2.1 Humanization Scheme 1 for Mouse Anti-mAb007

(1) Selection of Frameworks for Humanization of mAb007

For the mouse antibody mAb007, the light chain template for humanization was IGKV1-27 IGKV1-27*01 X63398 V-KAPPA F, and the heavy chain template for humanization was IGHV1-8 IGHV1-8*01 M99637 V_{H} F. After humanization, the humanized antibody hAb007 was obtained. The sequences of the humanized variable regions are as follows:

>hAb007 VH1-CDR grafted
(SEQ ID NO: 41)
QVQLVQSGAEVKKPGASVKVSCKASGYTFTDNYIHWVRQATGQGLEWMGW
IYPGSVNIKYNEKFKDRVTMTRNTSISTAYMELSSLRSEDTAVYYCARDI
SRYYFDYWGQGTLVTVSS
>hAb007 VL1-CDR grafted
(SEQ ID NO: 66)
DIQMTQSPSSLSASVGDRVTITCKSSQSLLNSRTRKNYLAWYQQKPGKAP
KLLIYWASTRESGVPSRFSGSGSGTDFTLTISSLQPEDFATYYCKQSFIL
RTFGGGTKVEIK

Note: The order is FR1-CDR1-FR2-CDR2-FR3-CDR3-FR4; the underlined sequences are the CDR sequences, and the ununderlined sequences are the FR sequences.
(2) the Back Mutation Design for hAb007 is as Follows:

| | | Donor | | | Donor | |
|---|---|---|---|---|---|---|
| | | Framework | SEQ | | Framework | SEQ |
| Antibody | Light Chain | Residue and | ID | Heavy | Residue and | ID |
| Name | Name | Back Mutation | NO. | Chain Name | Back Mutation | NO. |
| hAb007 | mAb007VL_hum1 | grafted | 66 | mAb007VH_hum1 | grafted | 41 |
| | mAb007VL_hum2 | A49S, S66D, | 67 | mAb007VH_hum2 | Y27S, R67K, | 42 |
| | | S69T, E110A | | | M70L, T91S | |
| | mAb007VL_hum3 | T5S, K48Q, A49S, | 68 | mAb007VH_hum3 | K12V, A40R, R72A, | 43 |
| | | S66D, S69T, P86A, | | | N73D, E82Q, R87S | |
| | | E110A | | | | |
| | mAb007VL_hum4 | Q3V, T5S, K48Q, | 69 | mAb007VH_hum4 | V11L, K12V, Y27S, A40R, | 44 |
| | | A49S, S66D, S69T, | | | M70L, R72A, N73D, S77T, | |
| | | L84V, V109L, E110A | | | E82Q, R87S, V93I | |

Note:
For example, A49S means that the A at position 49 was mutated back to S, numbering according to the natural order of the amino acid sequence. The “grafted” means CDRs of the mouse antibody were engrafted into the human germline FR region sequences.

(3) the Sequence Combinations for Humanization for hAb007 are as Follows:

| | mAb007VL_hum1 | mAb007VL_hum2 | mAb007VL_hum3 | mAb007VL_hum4 |
|---|---|---|---|---|
| mAb007VH_hum1 | hAb007-1 | hAb007-2 | hAb007-3 | hAb007-4 |
| mAb007VH_hum2 | hAb007-5 | hAb007-6 | hAb007-7 | hAb007-8 |
| mAb007VH_hum3 | hAb007-9 | hAb007-10 | hAb007-11 | hAb007-12 |
| mAb007VH_hum4 | hAb007-13 | hAb007-14 | hAb007-15 | hAb007-16 |

Note:
This table represents the sequences obtained by combining each mutation. For example, hAb007-5 means that there are four back mutations from light chain mAb007VL_hum1 and heavy chain mAb007VH_hum2 on the humanized antibody hAb007-5. The same applies to other sequences.

(4) the Specific Sequences for Humanization for hAb007 are as Follows:

>mAb007VL_hum1
(SEQ ID NO: 66)
DIQMTQSPSSLSASVGDRVTITCKSSQSLLNSRTRKNYLAWYQQKPGKAP
KLLIYWASTRESGVPSRFSGSGSGTDFTLTISSLQPEDFATYYCKQSFIL
RTFGGGTKVEIK
>mAb007VL_hum2
(SEQ ID NO: 67)
DIQMTQSPSSLSASVGDRVTITCKSSQSLLNSRTRKNYLAWYQQKPGKSP
KLLIYWASTRESGVPDRFTGSGSGTDFTLTISSLQPEDFATYYCKQSFIL
RTFGGGTKVAIK
>mAb007VL_hum3
(SEQ ID NO: 68)
DIQMSQSPSSLSASVGDRVTITCKSSQSLLNSRTRKNYLAWYQQKPGQSP
KLLIYWASTRESGVPDRFTGSGSGTDFTLTISSLQAEDFATYYCKQSFIL
RTFGGGTKVAIK
>mAb007VL hum4
(SEQ ID NO: 69)
DIVMSQSPSSLSASVGDRVTITCKSSQSLLNSRTRKNYLAWYQQKPGQSP
KLLIYWASTRESGVPDRFTGSGSGTDFTLTISSVQPEDFATYYCKQSFIL
RTFGGGTKLAIK
>mAb007VH hum1
(SEQ ID NO: 41)
QVQLVQSGAEVKKPGASVKVSCKASGYTFTDNYIHWVRQATGQGLEWMGW
IYPGSVNIKYNEKFKDRVTMTRNTSISTAYMELSSLRSEDTAVYYCARDI
SRYYFDYWGQGTLVTVSS
>mAb007VH hum2
(SEQ ID NO: 42)
QVQLVQSGAEVKKPGASVKVSCKASGSTFTDNYIHWVRQATGQGLEWMGW
IYPGSVNIKYNEKFKDKVTLTRNTSISTAYMELSSLRSEDSAVYYCARDI
SRYYFDYWGQGTLVTVSS
>mAb007VH hum3
(SEQ ID NO: 43)
QVQLVQSGAEVVKPGASVKVSCKASGYTFTDNYIHWVRQRTGQGLEWMGW
IYPGSVNIKYNEKFKDRVTMTADTSISTAYMQLSSLSSEDTAVYYCARDI
SRYYFDYWGQGTLVTVSS
>mAb007VH hum4
(SEQ ID NO: 44)
QVQLVQSGAELVKPGASVKVSCKASGSTFTDNYIHWVRQRTGQGLEWMGW
IYPGSVNIKYNEKFKDRVTLTADTSITTAYMQLSSLSSEDTAIYYCARDI
SRYYFDYWGQGTLVTVSS

### 3.2.2 Humanization Scheme 2 for Mouse Anti-mAb007

(1) Selection of Frameworks for Humanization of mAb007

For the mouse antibody mAb007, the light chain template for humanization was IGKV4-1 IGKV4-1*01 Z0023 V-KAPPA, and the heavy chain template for humanization was IGHV1-3 IGHV1-3*01 X62109 VH. After humanization, the humanized antibody hAb007 was obtained. The sequences of the humanized variable regions are as follows:

>hAb007 VH2-CDR grafted
(SEQ ID NO: 45)
QVQLVQSGAEVKKPGASVKVSCKASGSTFTDNYIHWVRQAPGQRLEWMGW
IYPGSVNIKYNEKFKDRVTITRDTSASTAYMELSSLRSEDTAVYYCARDI
SRYYFDYWGQGTTVTVSS
>hAb007 VL2-CDR grafted
(SEQ ID NO: 70)
DIVMTQSPDSLAVSLGERATINCKSSQSLLNSRTRKNYLAWYQQKPGQPP
KLLIYWASTRESGVPDRFSGSGSGTDFTLTISSLQAEDVAVYYCKQSFIL
RTFGGGTKLEIK

Note: The order is FR1-CDR1-FR2-CDR2-FR3-CDR3-FR4; the underlined sequences are the CDR sequences, and the ununderlined sequences are the FR sequences.

(2) the Back Mutation Design for hAb007 is as Follows:

| | | Donor | | | Donor | |
|---|---|---|---|---|---|---|
| | | Framework | SEQ | | Framework | SEQ |
| Antibody | Light Chain | Residue and | ID | Heavy | Residue and | ID |
| Name | Name | Back Mutation | NO. | Chain Name | Back Mutation | NO. |
| hAb007 | mAb007VL_hum5 | grafted | 70 | mAb007VH_hum5 | grafted | 45 |
| | mAb007VL_hum6 | S69T | 71 | mAb007VH_hum6 | M48I, R72A | 46 |
| | mAb007VL_hum7 | I21M | 72 | mAb007VH_hum7 | M48I, V68A, | 47 |
| | | | | | R72A | |
| | mAb007VL_hum8 | S69T, I21M | 73 | mAb007VH_hum8 | R38K, M48I, | 48 |
| | | | | | V68A, R72A, S77T | |

Note:
For example, S69T means that the S at position 69 was mutated back to T, numbering according to the natural order of the amino acid sequence. The “grafted” means CDRs of the mouse antibody were engrafted into the human germline FR region sequences.

(3) the Sequence Combinations for Humanization for hAb007 are as Follows:

| | mAb007VL_hum5 | mAb007VL_hum6 | mAb007VL_hum7 | mAb007VL_hum8 |
|---|---|---|---|---|
| mAb007VH_hum5 | hAb007-17 | hAb007-18 | hAb007-19 | hAb007-20 |
| mAb007VH_hum6 | hAb007-21 | hAb007-22 | hAb007-23 | hAb007-24 |
| mAb007VH_hum7 | hAb007-25 | hAb007-26 | hAb007-27 | hAb007-28 |
| mAb007VH_hum8 | hAb007-29 | hAb007-30 | hAb007-31 | hAb007-32 |

Note:
This table represents the sequences obtained by combining each mutation. For example, hAb007-22 means that there are three back mutations from light chain mAb007VL_hum6 and heavy chain mAb007VH_hum6 on the humanized antibody hAb007-22. The same applies to other sequences.

(4) the Specific Sequences for Humanization for hAb007 are as Follows:

>mAb007VL_hum5
(SEQ ID NO: 70)
DIVMTQSPDSLAVSLGERATINCKSSQSLLNSRTRKNYLAWYQQKPGQPP
KLLIYWASTRESGVPDRFSGSGSGTDFTLTISSLQAEDVAVYYCKQSFIL
RTFGGGTKLEIK
>mAb007VL_hum6
(SEQ ID NO: 71)
DIVMTQSPDSLAVSLGERATINCKSSQSLLNSRTRKNYLAWYQQKPGQPP
KLLIYWASTRESGVPDRFTGSGSGTDFTLTISSLQAEDVAVYYCKQSFIL
RTFGGGTKLEIK
>mAb007VL_hum7
(SEQ ID NO: 72)
DIVMTQSPDSLAVSLGERATMNCKSSQSLLNSRTRKNYLAWYQQKPGQPP
KLLIYWASTRESGVPDRFSGSGSGTDFTLTISSLQAEDVAVYYCKQSFIL
RTFGGGTKLEIK
>mAb007VL_hum8
(SEQ ID NO: 73)
DIVMTQSPDSLAVSLGERATMNCKSSQSLLNSRTRKNYLAWYQQKPGQPP
KLLIYWASTRESGVPDRFTGSGSGTDFTLTISSLQAEDVAVYYCKQSFIL
RTFGGGTKLEIK
>mAb007VH_hum5
(SEQ ID NO: 45)
QVQLVQSGAEVKKPGASVKVSCKASGSTFTDNYIHWVRQAPGQRLEWMGW
IYPGSVNIKYNEKFKDRVTITRDTSASTAYMELSSLRSEDTAVYYCARDI
SRYYFDYWGQGTTVTVSS
>mAb007VH_hum6
(SEQ ID NO: 46)
QVQLVQSGAEVKKPGASVKVSCKASGSTFTDNYIHWVRQAPGQRLEWIGW
IYPGSVNIKYNEKFKDRVTITADTSASTAYMELSSLRSEDTAVYYCARDI
SRYYFDYWGQGTTVTVSS
>mAb007VH_hum7
(SEQ ID NO: 47)
QVQLVQSGAEVKKPGASVKVSCKASGSTFTDNYIHWVRQAPGQRLEWIGW
IYPGSVNIKYNEKFKDRATITADTSASTAYMELSSLRSEDTAVYYCARDI
SRYYFDYWGQGTTVTVSS
>mAb007VH_hum8
(SEQ ID NO: 48)
QVQLVQSGAEVKKPGASVKVSCKASGSTFTDNYIHWVKQAPGQRLEWIGW
IYPGSVNIKYNEKFKDRATITADTSATTAYMELSSLRSEDTAVYYCARDI
SRYYFDYWGQGTTVTVSS

### 3.3 Humanization of Mouse Anti-mAb008

### 3.3.1 Humanization Scheme 1 for Mouse Anti-mAb008

(1) Selection of Frameworks for Humanization of mAb008

For the mouse antibody mAb008, the light chain template for humanization was IIGKV2-30 IGKV2-30*01 X63403 V-KAPPA F, and the heavy chain template for humanization was IGHV1-8 IGHV1-8*01 M99637 VH F. After humanization, the humanized antibody hAb008 was obtained. The sequences of the humanized variable regions are as follows:

>hAb008 VH-CDR grafted
(SEQ ID NO: 49)
QVQLVQSGAEVKKPGSSVKVSCKASGYTFTDYTVNWVRQAPGQGLEWMGY
INPFNSYTKYNQKFKDRVTITADESTSTAYMELSSLRSEDTAVYYCANSV
STYWGQGTLVTVSS
>hAb008 VL-CDR grafted
(SEQ ID NO: 74)
DVVMTQSPLSLPVTLGQPASISCKSSQSLLDSDGKTYLNWFQQRPGQSPR
RLIYLVSKLNSGVPDRFSGSGSGTDFTLKISRVEAEDVGVYYCWQGTHFP
RTFGGGTKVEIK

Note: The order is FR1-CDR1-FR2-CDR2-FR3-CDR3-FR4; the underlined sequences are the CDR sequences, and the ununderlined sequences are the FR sequences.

(2) the Back Mutation Design for hAb008 is as Follows:

| | | Donor | | | Donor | |
|---|---|---|---|---|---|---|
| | | Framework | SEQ | | Framework | SEQ |
| Antibody | Light Chain | Residue and | ID | Heavy | Residue and | ID |
| Name | Name | Back Mutation | NO. | Chain Name | Back Mutation | NO. |
| hAb008 | mAb008VL_hum1 | grafted | 74 | mAb008VH_hum1 | grafted | 49 |
| | mAb008VL_hum2 | F41L, | 75 | mAb008VH_hum2 | K12A, A40R | 50 |
| | mAb008VL_hum3 | F41L, V54Q | 76 | mAb008VH_hum3 | K12A, A40R, | 51 |
| | | | | | R87T | |
| | | | | mAb008VH_hum4 | K12A, A24T, A40R, | 52 |
| | | | | | I70L, R87T | |
| | | | | mAb008VH_hum5 | K12A, A24T, A40R, | 53 |
| | | | | | I70L, E74K, E82Q, R87T | |

Note:
For example, F41L means that the F at position 41 was mutated back to L, numbering according to the natural order of the amino acid sequence. The “grafted” means CDRs of the mouse antibody were engrafted into the human germline FR region sequences.

(3) The Sequence Combinations for Humanization for hAb008 are as Follows:

| | mAb008VL_hum1 | mAb008VL_hum2 | mAb008VL_hum3 |
|---|---|---|---|
| mAb008VH_hum1 | hAb008-1 | hAb008-2 | hAb008-3 |
| mAb008VH_hum2 | hAb008-4 | hAb008-5 | hAb008-6 |
| mAb008VH_hum3 | hAb008-7 | hAb008-8 | hAb008-9 |
| mAb008VH_hum4 | hAb008-10 | hAb008-11 | hAb008-12 |
| mAb008VH_hum5 | hAb008-13 | hAb008-14 | hAb008-15 |

Note:
This table represents the sequences obtained by combining each mutation. For example, hAb008-5 means that there are three back mutations from light chain mAb008VL_hum2 and heavy chain mAb007VH_hum2 on the humanized antibody hAb008-5. The same applies to other sequences.

(4) the Specific Sequences for Humanization for hAb008 are as Follows:

>mAb008VL_hum1
(SEQ ID NO: 74)
DVVMTQSPLSLPVTLGQPASISCKSSQSLLDSDGKTYLNWFQQRPGQSPR
RLIYLVSKLNSGVPDRFSGSGSGTDFTLKISRVEAEDVGVYYCWQGTHFP
RTFGGGTKVEIK
>mAb008VL_hum2
(SEQ ID NO: 75)
DVVMTQSPLSLPVTLGQPASISCKSSQSLLDSDGKTYLNWLQQRPGQSPR
RLIYLVSKLNSGVPDRFSGSGSGTDFTLKISRVEAEDVGVYYCWQGTHFP
RTFGGGTKVEIK
>mAb008VL_hum3
(SEQ ID NO: 76)
DVVMTQSPLSLPVTLGQPASISCKSSQSLLDSDGKTYLNWLQQRPGQSPR
RLIQLVSKLNSGVPDRFSGSGSGTDFTLKISRVEAEDVGVYYCWQGTHFP
RTFGGGTKVEIK
>mAb008VH_hum1
(SEQ ID NO: 49)
QVQLVQSGAEVKKPGSSVKVSCKASGYTFTDYTVNWVRQAPGQGLEWMGY
INPFNSYTKYNQKFKDRVTITADESTSTAYMELSSLRSEDTAVYYCANSV
STYWGQGTLVTVSS
>mAb008VH_hum2
(SEQ ID NO: 50)
QVQLVQSGAEVAKPGSSVKVSCKASGYTFTDYTVNWVRQRPGQGLEWMGY
INPFNSYTKYNQKFKDRVTITADESTSTAYMELSSLRSEDTAVYYCANSV
STYWGQGTLVTVSS
>mAb008VH_hum3
(SEQ ID NO: 51)
QVQLVQSGAEVAKPGSSVKVSCKASGYTFTDYTVNWVRQRPGQGLEWMGY
INPFNSYTKYNQKFKDRVTITADESTSTAYMELSSLTSEDTAVYYCANSV
STYWGQGTLVTVSS
>mAb008VH_hum4
(SEQ ID NO: 52)
QVQLVQSGAEVAKPGSSVKVSCKTSGYTFTDYTVNWVRQRPGQGLEWMGY
INPFNSYTKYNQKFKDRVTLTADESTSTAYMELSSLTSEDTAVYYCANSV
STYWGQGTLVTVSS
>mAb008VH_hum5
(SEQ ID NO: 53)
QVQLVQSGAEVAKPGSSVKVSCKTSGYTFTDYTVNWVRQRPGQGLEWMGY
INPFNSYTKYNQKFKDRVTLTADKSTSTAYMQLSSLTSEDTAVYYCANSV
STYWGQGTLVTVSS

### 3.3.2 Humanization Scheme 2 for Hybridoma Mouse Anti-mAb008

(1) Selection of Frameworks for Humanization of mAb008

For the mouse antibody mAb008, the light chain template for humanization was IIGKV2-30 IGKV2-30*01 X63403 V-KAPPA F, and the heavy chain template for humanization was IGHV1-3 IGHV1-3*01 X62109 VH. After humanization, the humanized antibody hAb008 was obtained. The sequences of the humanized variable regions are as follows:

>hAb008 VH2-CDR grafted
(SEQ ID NO: 54)
QVQLVQSGAEVKKPGASVKVSCKASGYTFTDYTVNWVRQAPGQRLEWMGY
INPFNSYTKYNQKFKDRVTITRDTSASTAYMELSSLRSEDTAVYYCARSV
STYWGQGTTVTVSS
>hAb008 VL2-CDR grafted
(SEQ ID NO: 77)
DVVMTQSPLSLPVTLGQPASISCKSSQSLLDSDGKTYLNWFQQRPGQSPR
RLIYLVSKLNSGVPDRFSGSGSGTDFTLKISRVEAEDVGVYYCWQGTHFP
RTFGGGTKLEIK

Note: The order is FR1-CDR1-FR2-CDR2-FR3-CDR3-FR4; the underlined sequences are the CDR sequences, and the ununderlined sequences are the FR sequences.

(2) the Back Mutation Design for hAb008 is as Follows:

| | | Donor | | | Donor | |
|---|---|---|---|---|---|---|
| | | Framework | SEQ | | Framework | SEQ |
| Antibody | Light Chain | Residue and | ID | Heavy | Residue and | ID |
| Name | Name | Back Mutation | NO. | Chain Name | Back Mutation | NO. |
| hAb008 | mAb008VL_hum4 | grafted | 77 | mAb008VH_hum6 | grafted | 54 |
| | mAb008VL_hum5 | Y54Q, Y91F | 78 | mAb008VH_hum7 | M48I, R72A, | 55 |
| | | | | | R98N | |
| | mAb008VL_hum6 | F41L, Y54Q, | 79 | mAb008VH_hum8 | M48I, V68A, | 56 |
| | | Y91F | | | R72A, Y95F, R98N | |
| | mAb008VL_hum7 | F41L, Q42L, | 80 | mAb008VH_hum9 | R38K, M48I, V68A, | 57 |
| | | Y54Q, Y91F | | | R72A, Y95F, R98N | |

Note:
For example, Y54Q means that the Y at position 54 was mutated back to Q, numbering according to the natural order of the amino acid sequence. The “grafted” means CDRs of the mouse antibody were engrafted into the human germline FR region sequences.

(3) The Sequence Combinations for Humanization for hAb008 are as Follows:

| | mAb008VL_hum4 | mAb008VL_hum5 | mAb008VL_hum6 | mAb008VL_hum7 |
|---|---|---|---|---|
| mAb008VH_hum6 | hAb008-16 | hAb008-17 | hAb008-18 | hAb008-19 |
| mAb008VH_hum7 | hAb008-20 | hAb008-21 | hAb008-22 | hAb008-23 |
| mAb008VH_hum8 | hAb008-24 | hAb008-25 | hAb008-26 | hAb008-27 |
| mAb008VH_hum9 | hAb008-28 | hAb008-29 | hAb008-30 | hAb008-31 |

Note:
This table represents the sequences obtained by combining each mutation. For example, hAb008-22 means that there are six back mutations from light chain mAb008VL_hum6 and heavy chain mAb007VH_hum7 on the humanized antibody hAb008-22. The same applies to other sequences.

(4) the Specific Sequences for Humanization for hAb008 are as Follows:

>mAb008VL_hum4
(SEQ ID NO: 77)
DVVMTQSPLSLPVTLGQPASISCKSSQSLLDSDGKTYLNWFQQRPGQSPR
RLIYLVSKLNSGVPDRFSGSGSGTDFTLKISRVEAEDVGVYYCWQGTHFP
RTFGGGTKLEIK
>mAb008VL_hum5
(SEQ ID NO: 78)
DVVMTQSPLSLPVTLGQPASISCKSSQSLLDSDGKTYLNWFQQRPGQSPR
RLIQLVSKLNSGVPDRFSGSGSGTDFTLKISRVEAEDVGVFYCWQGTHFP
RTFGGGTKLEIK
>mAb008VL_hum6
(SEQ ID NO: 79)
DVVMTQSPLSLPVTLGQPASISCKSSQSLLDSDGKTYLNWLQQRPGQSPR
RLIQLVSKLNSGVPDRFSGSGSGTDFTLKISRVEAEDVGVFYCWQGTHFP
RTFGGGTKLEIK
>mAb008VL_hum7
(SEQ ID NO: 80)
DVVMTQSPLSLPVTLGQPASISCKSSQSLLDSDGKTYLNWLLQRPGQSPR
RLIQLVSKLNSGVPDRFSGSGSGTDFTLKISRVEAEDVGVFYCWQGTHFP
RTFGGGTKLEIK
>mAb008VH_hum6
(SEQ ID NO: 54)
QVQLVQSGAEVKKPGASVKVSCKASGYTFTDYTVNWVRQAPGQRLEWMGY
INPFNSYTKYNQKFKDRVTITRDTSASTAYMELSSLRSEDTAVYYCARSV
STYWGQGTTVTVSS
>mAb008VH_hum7
(SEQ ID NO: 55)
QVQLVQSGAEVKKPGASVKVSCKASGYTFTDYTVNWVRQAPGQRLEWIGY
INPFNSYTKYNQKFKDRVTITADTSASTAYMELSSLRSEDTAVYYCANSV
STYWGQGTTVTVSS
>mAb008VH_hum8
(SEQ ID NO: 56)
QVQLVQSGAEVKKPGASVKVSCKASGYTFTDYTVNWVRQAPGQRLEWIGY
INPFNSYTKYNQKFKDRATITADTSASTAYMELSSLRSEDTAVYFCANSV
STYWGQGTTVTVSS
>mAb008VH_hum9
(SEQ ID NO: 57)
QVQLVQSGAEVKKPGASVKVSCKASGYTFTDYTVNWVKQAPGQRLEWIGY
INPFNSYTKYNQKFKDRATITADTSASTAYMELSSLRSEDTAVYFCANSV
STYWGQGTTVTVSS

### 3.3.3 Hotspot Mutation of Hybridoma Mouse Anti-mAb008 Chimeric Antibody

A hotspot of the antibody mAb008 was subjected to point mutation. The light chain of the antibody mAb008 had one mutable site, and the G at position 34 of the light chain was mutated back to A, generating the hotspot mutated chimeric antibody CAb008DA.

>mAb008VL DA
(SEQ ID NO: 81)
DVVMTQTPLTLSVTIGQPASISCKSSQSLLDSDAKTYLNWLLQRPGQSPK
RLIQLVSKLNSGVPDRFTGSGSGTDFTLKISRVEAEDLGIFYCWQGTHFP
RTFGGGTKLEIK
>mAb008VH or CAb008VH
(SEQ ID NO: 89)
QVQLQQSGAELARPGASVKMSCKTSGYTFTDYTVNWVKQRPGQGLEWIGY
INPFNSYTKYNQKFKDKATLTADKSSSTAYMQLSSLTSEDSAVYFCANSV
STYWGQGTTLTVSS
>mAb008VL or CAb008VL
(SEQ ID NO: 90)
DVVMTQTPLTLSVTIGQPASISCKSSQSLLDSDGKTYLNWLLQRPGQSPK
RLIQLVSKLNSGVPDRFTGSGSGTDFTLKISRVEAEDLGIFYCWQGTHFP
RTFGGGTKLEIK

### 3.4 Hotspot Mutations of Hybridoma Mouse Anti-mAb009 Chimeric Antibody

Hotspots of the antibody mAb009 were subjected to point mutation. The light chain of the antibody mAb009 had two mutable sites, and the G at position 32 of the light chain was mutated back to A, generating the hotspot mutated chimeric antibody CAb009DADG.

>mAb009VL DADG
(SEQ ID NO: 82)
DVVMTQTPLTLSVTIGQPASISCKSSQSLLDADGKTYLNWLLQRPGQSPK
RLIQLVSKLNSGVPDRFTGSGSGTDFTLKISRVEAEDLGIYYCWQGTHFP
RTFGGGTKLEIK
>mAb009VH or CAb009VH
(SEQ ID NO: 85)
QVQLQQSGAELTRPGASVKMSCKASGYTFTDYTIHWVKQRPGQGLEWIGY
INPFSDYTKYNQKFKDKATLTADKSSSTAYIQLNSLTSEDSAVYYCANSV
SIYWGQGTTLTVSS
>mAb009VL or CAb009VL
(SEQ ID NO: 86)
DVVMTQTPLTLSVTIGQPASISCKSSQSLLDGDGKTYLNWLLQRPGQSPK
RLIQLVSKLNSGVPDRFTGSGSGTDFTLKISRVEAEDLGIYYCWQGTHFP
RTFGGGTKLEIK

The sequences of the antibodies tested in this disclosure are summarized in the following table:

TABLE 6
Sequence No. of antibodies as tested

| | SEQ ID NO: | | | | | | | |
|---|---|---|---|---|---|---|---|---|
| | | | | | | | Heavy | Light |
| | Heavy | Heavy | Heavy | Light | Light | Light | Chain | Chain |
| | Chain | Chain | Chain | Chain | Chain | Chain | Variable | Variable |
| Antibody No. | CDR1 | CDR2 | CDR3 | CDR1 | CDR2 | CDR3 | Region | Region |
| CAb001 | 1 | 18 | 8 | 24 | 14 | 31 | 87 | 88 |
| hAb001-1 | 1 | 18 | 8 | 24 | 14 | 31 | 33 | 58 |
| hAb001-2 | 1 | 18 | 8 | 25 | 14 | 31 | 33 | 59 |
| hAb001-3 | 1 | 18 | 8 | 24 | 14 | 31 | 33 | 60 |
| hAb001-4 | 1 | 18 | 8 | 25 | 14 | 31 | 33 | 61 |
| hAb001-5 | 1 | 19 | 8 | 24 | 14 | 31 | 34 | 58 |
| hAb001-6 | 1 | 19 | 8 | 25 | 14 | 31 | 34 | 59 |
| hAb001-7 | 1 | 19 | 8 | 24 | 14 | 31 | 34 | 60 |
| hAb001-8 | 1 | 19 | 8 | 25 | 14 | 31 | 34 | 61 |
| hAb001-9 | 1 | 20 | 8 | 24 | 14 | 31 | 35 | 58 |
| hAb001-10 | 1 | 20 | 8 | 25 | 14 | 31 | 35 | 59 |
| hAb001-11 | 1 | 20 | 8 | 24 | 14 | 31 | 35 | 60 |
| hAb001-12 | 1 | 20 | 8 | 25 | 14 | 31 | 35 | 61 |
| hAb001-13 | 1 | 21 | 8 | 24 | 14 | 31 | 36 | 58 |
| hAb001-14 | 1 | 21 | 8 | 25 | 14 | 31 | 36 | 59 |
| hAb001-15 | 1 | 21 | 8 | 24 | 14 | 31 | 36 | 60 |
| hAb001-16 | 1 | 21 | 8 | 25 | 14 | 31 | 36 | 61 |
| hAb001-17 | 1 | 18 | 8 | 24 | 14 | 31 | 37 | 62 |
| hAb001-18 | 1 | 18 | 8 | 24 | 14 | 31 | 37 | 63 |
| hAb001-19 | 1 | 18 | 8 | 24 | 14 | 31 | 37 | 64 |
| hAb001-20 | 1 | 18 | 8 | 24 | 14 | 31 | 37 | 65 |
| hAb001-21 | 1 | 18 | 8 | 24 | 14 | 31 | 38 | 62 |
| hAb001-22 | 1 | 18 | 8 | 24 | 14 | 31 | 38 | 63 |
| hAb001-23 | 1 | 18 | 8 | 24 | 14 | 31 | 38 | 64 |
| hAb001-24 | 1 | 18 | 8 | 24 | 14 | 31 | 38 | 65 |
| hAb001-25 | 1 | 18 | 8 | 24 | 14 | 31 | 39 | 62 |
| hAb001-26 | 1 | 18 | 8 | 24 | 14 | 31 | 39 | 63 |
| hAb001-27 | 1 | 18 | 8 | 24 | 14 | 31 | 39 | 64 |
| hAb001-28 | 1 | 18 | 8 | 24 | 14 | 31 | 39 | 65 |
| hAb001-29 | 1 | 18 | 8 | 24 | 14 | 31 | 40 | 62 |
| hAb001-30 | 1 | 18 | 8 | 24 | 14 | 31 | 40 | 63 |
| hAb001-31 | 1 | 18 | 8 | 24 | 14 | 31 | 40 | 64 |
| hAb001-32 | 1 | 18 | 8 | 24 | 14 | 31 | 40 | 65 |
| CAb007 | 2 | 6 | 9 | 26 | 14 | 32 | 91 | 92 |
| hAb007-1 | 2 | 6 | 9 | 26 | 14 | 32 | 41 | 66 |
| hAb007-2 | 2 | 6 | 9 | 26 | 14 | 32 | 41 | 67 |
| hAb007-3 | 2 | 6 | 9 | 26 | 14 | 32 | 41 | 68 |
| hAb007-4 | 2 | 6 | 9 | 26 | 14 | 32 | 41 | 69 |
| hAb007-5 | 2 | 6 | 9 | 26 | 14 | 32 | 42 | 66 |
| hAb007-6 | 2 | 6 | 9 | 26 | 14 | 32 | 42 | 67 |
| hAb007-7 | 2 | 6 | 9 | 26 | 14 | 32 | 42 | 68 |
| hAb007-8 | 2 | 6 | 9 | 26 | 14 | 32 | 42 | 69 |
| hAb007-9 | 2 | 6 | 9 | 26 | 14 | 32 | 43 | 66 |
| hAb007-10 | 2 | 6 | 9 | 26 | 14 | 32 | 43 | 67 |
| hAb007-11 | 2 | 6 | 9 | 26 | 14 | 32 | 43 | 68 |
| hAb007-12 | 2 | 6 | 9 | 26 | 14 | 32 | 43 | 69 |
| hAb007-13 | 2 | 6 | 9 | 26 | 14 | 32 | 44 | 66 |
| hAb007-14 | 2 | 6 | 9 | 26 | 14 | 32 | 44 | 67 |
| hAb007-15 | 2 | 6 | 9 | 26 | 14 | 32 | 44 | 68 |
| hAb007-16 | 2 | 6 | 9 | 26 | 14 | 32 | 44 | 69 |
| hAb007-17 | 2 | 6 | 9 | 26 | 14 | 32 | 45 | 70 |
| hAb007-18 | 2 | 6 | 9 | 26 | 14 | 32 | 45 | 71 |
| hAb007-19 | 2 | 6 | 9 | 26 | 14 | 32 | 45 | 72 |
| hAb007-20 | 2 | 6 | 9 | 26 | 14 | 32 | 45 | 73 |
| hAb007-21 | 2 | 6 | 9 | 26 | 14 | 32 | 46 | 70 |
| hAb007-22 | 2 | 6 | 9 | 26 | 14 | 32 | 46 | 71 |
| hAb007-23 | 2 | 6 | 9 | 26 | 14 | 32 | 46 | 72 |
| hAb007-24 | 2 | 6 | 9 | 26 | 14 | 32 | 46 | 73 |
| hAb007-25 | 2 | 6 | 9 | 26 | 14 | 32 | 47 | 70 |
| hAb007-26 | 2 | 6 | 9 | 26 | 14 | 32 | 47 | 71 |
| hAb007-27 | 2 | 6 | 9 | 26 | 14 | 32 | 47 | 72 |
| hAb007-28 | 2 | 6 | 9 | 26 | 14 | 32 | 47 | 73 |
| hAb007-29 | 2 | 6 | 9 | 26 | 14 | 32 | 48 | 70 |
| hAb007-30 | 2 | 6 | 9 | 26 | 14 | 32 | 48 | 71 |
| hAb007-31 | 2 | 6 | 9 | 26 | 14 | 32 | 48 | 72 |
| hAb007-32 | 2 | 6 | 9 | 26 | 14 | 32 | 48 | 73 |
| CAb008 | 3 | 22 | 10 | 27 | 15 | 17 | 89 | 90 |
| CAb008DA | 3 | 22 | 10 | 28 | 15 | 17 | 89 | 81 |
| hAb008-1 | 3 | 22 | 10 | 27 | 15 | 17 | 49 | 74 |
| hAb008-2 | 3 | 22 | 10 | 27 | 15 | 17 | 49 | 75 |
| hAb008-3 | 3 | 22 | 10 | 27 | 15 | 17 | 49 | 76 |
| hAb008-4 | 3 | 22 | 10 | 27 | 15 | 17 | 50 | 74 |
| hAb008-5 | 3 | 22 | 10 | 27 | 15 | 17 | 50 | 75 |
| hAb008-6 | 3 | 22 | 10 | 27 | 15 | 17 | 50 | 76 |
| hAb008-7 | 3 | 22 | 10 | 27 | 15 | 17 | 51 | 74 |
| hAb008-8 | 3 | 22 | 10 | 27 | 15 | 17 | 51 | 75 |
| hAb008-9 | 3 | 22 | 10 | 27 | 15 | 17 | 51 | 76 |
| hAb008-10 | 3 | 22 | 10 | 27 | 15 | 17 | 52 | 74 |
| hAb008-11 | 3 | 22 | 10 | 27 | 15 | 17 | 52 | 75 |
| hAb008-12 | 3 | 22 | 10 | 27 | 15 | 17 | 52 | 76 |
| hAb008-13 | 3 | 22 | 10 | 27 | 15 | 17 | 53 | 74 |
| hAb008-14 | 3 | 22 | 10 | 27 | 15 | 17 | 53 | 75 |
| hAb008-15 | 3 | 22 | 10 | 27 | 15 | 17 | 53 | 76 |
| hAb008-16 | 3 | 22 | 10 | 27 | 15 | 17 | 54 | 77 |
| hAb008-17 | 3 | 22 | 10 | 27 | 15 | 17 | 54 | 78 |
| hAb008-18 | 3 | 22 | 10 | 27 | 15 | 17 | 54 | 79 |
| hAb008-19 | 3 | 22 | 10 | 27 | 15 | 17 | 54 | 80 |
| hAb008-20 | 3 | 22 | 10 | 27 | 15 | 17 | 55 | 77 |
| hAb008-21 | 3 | 22 | 10 | 27 | 15 | 17 | 55 | 78 |
| hAb008-22 | 3 | 22 | 10 | 27 | 15 | 17 | 55 | 79 |
| hAb008-23 | 3 | 22 | 10 | 27 | 15 | 17 | 55 | 80 |
| hAb008-24 | 3 | 22 | 10 | 27 | 15 | 17 | 56 | 77 |
| hAb008-25 | 3 | 22 | 10 | 27 | 15 | 17 | 56 | 78 |
| hAb008-26 | 3 | 22 | 10 | 27 | 15 | 17 | 56 | 79 |
| hAb008-27 | 3 | 22 | 10 | 27 | 15 | 17 | 56 | 80 |
| hAb008-28 | 3 | 22 | 10 | 27 | 15 | 17 | 57 | 77 |
| hAb008-29 | 3 | 22 | 10 | 27 | 15 | 17 | 57 | 78 |
| hAb008-30 | 3 | 22 | 10 | 27 | 15 | 17 | 57 | 79 |
| hAb008-31 | 3 | 22 | 10 | 27 | 15 | 17 | 57 | 80 |
| CAb009 | 4 | 23 | 11 | 29 | 15 | 17 | 85 | 86 |
| CAb009DADG | 4 | 23 | 11 | 30 | 15 | 17 | 85 | 82 |

>CAb001 VH
(SEQ ID NO: 87)
QVQLQQSGPELVKPGASVKISCKASGYSFTNYYMHWVKQRPGQGLEWIGW
TYPGNNNIKYNEKFKGKATLTADTSSSTAYMQLSSLTSEDSAVYYCARDG
YGYYFFDYWGQGIILTVSS
>CAb001 VL
(SEQ ID NO: 88)
DIVMSQSPSSLAVSAGEKVTMSCKSSQSLLNNRTRKNYLAWYQQKPGQSP
KLLIYWASTRESGVPDRFTGSGSGTDFTLTISSVQAEDLAIYYCKQSYTL
RTFGGGTKLAIK
>CAb007 VH
(SEQ ID NO: 91)
QVQLQQSGPELVKPGASVKISCKASGSTFTDNYIHWVKQRPGQGLEWIGW
IYPGSVNIKYNEKFKDKATLTADTSSTTAYMQLSSLSSEDSAIYYCARDI
SRYYFDYWGQGTTLTVSS
>CAb007 VL
(SEQ ID NO: 92)
DIVMSQSPSSLAVSAGERVTMNCKSSQSLLNSRTRKNYLAWYQQKPGQSP
KLLIYWASTRESGVPDRFTGSGSGTDFTLTISSVQAEDLAVYYCKQSFIL
RTFGGGTKLAIK

### Example 5. Binding of Humanized or Hotspot Mutated Variants of CD3 Antibodies to Jurkat Cells

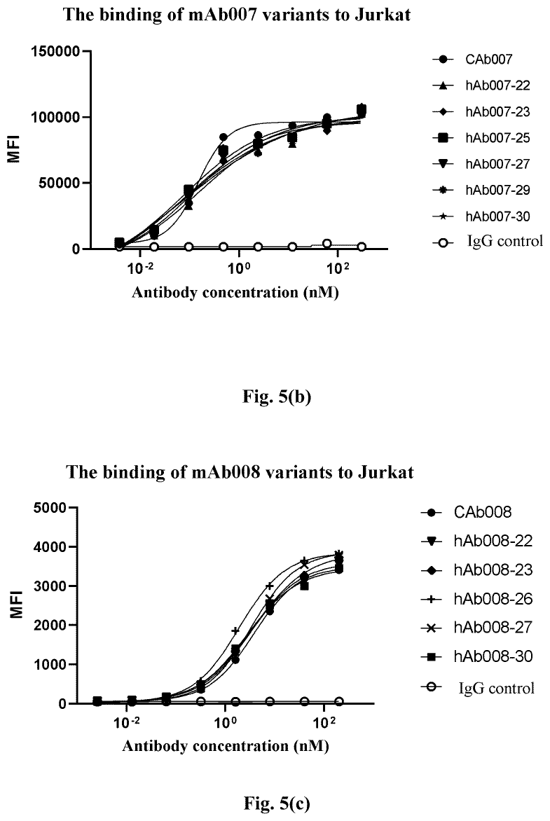

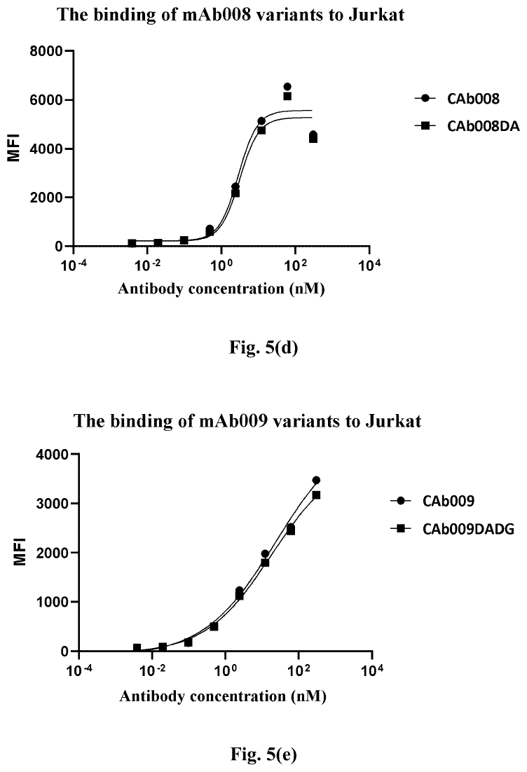

Jurkat cells were amplificatorily cultured to 3×10⁶/ml in a T-175 cell culture flask, the culture medium was removed by centrifugation, and the cells were washed twice with PBS buffer (purchased from Invitrogen). After cell counting, the cells were diluted to 2×10⁶ cells/ml with PBS buffer, added with 1% BSA blocking solution, where the percentage was a mass percentage, incubated on ice for 30 minutes, and then washed twice with PBS buffer by centrifugation. The collected cells were suspended to 2×10⁶ cells/mL with FACS buffer (PBS+1% BSA, where the percentage was a mass percentage), and added to a 96-well FACS reaction plate at 100 μl per well, and samples of the purified CD3 antibodies obtained in Example 4 to be tested were added at 100 μl per well, and incubated at 4° C. for 2 hours. The plate was washed twice with FACS buffer by centrifugation, added with 100 μl per well of fluorescent (Alexa 488) labeled secondary antibody (purchased from Jackson), and incubated at 4° C. for 1 hour. After washing 3 times by centrifugation with FACS buffer, the cells were suspended with 100 μl of FACS buffer, and detected and analyzed using BD FACS LSRFortessa (purchased from BD). The results are shown in FIG. 5, where the IgG control is human IgG1 (hIgG).

### Example 6. Effect of Antibodies on Activation of Total Human T Cells

Total human T cells were extracted using the total human T cell isolation kit (EasySep™ Direct Human T Cell Isolation Kit, purchased from Stemcell, catalog number 19661), and washed 3 times with complete medium. T cells were mixed evenly with diluted CD3 antibodies in a 96-well plate and then cultured at 37° C. for 3 days. After 3 days, the culture supernatant was collected and the human IFN-γ level in the culture supernatant was detected using an ELISA kit (IFN gamma Human Uncoated ELISA Kit, purchased from Invitrogen, catalog number 88-7316-88). The cultured cells were washed once with PBS and then added with a labeled anti-human CD25 flow cytometry antibody (PE-Cy7 Mouse Anti-Human CD25, purchased from BD Pharmingen, catalog number 557741) for staining in the dark for 30 minutes and then washed twice with PBS. The expression of CD25 on human T cells was analyzed using FACS (BD FACS LSRFortessa, purchased from BD).

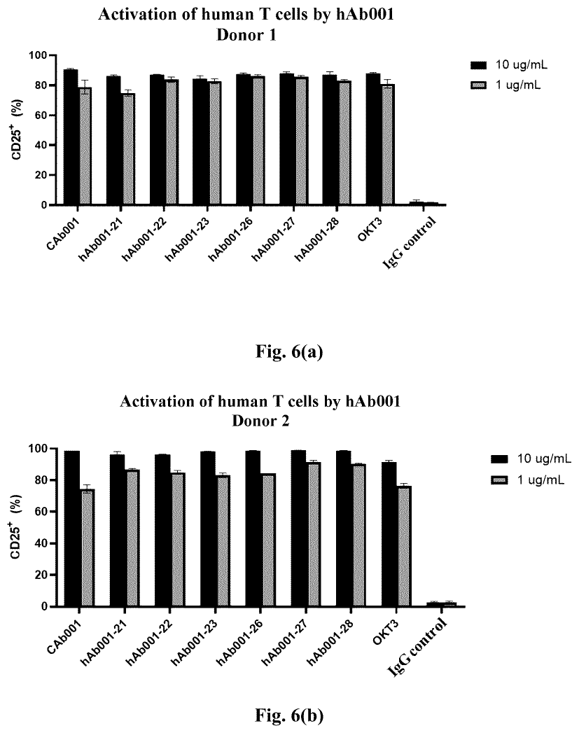

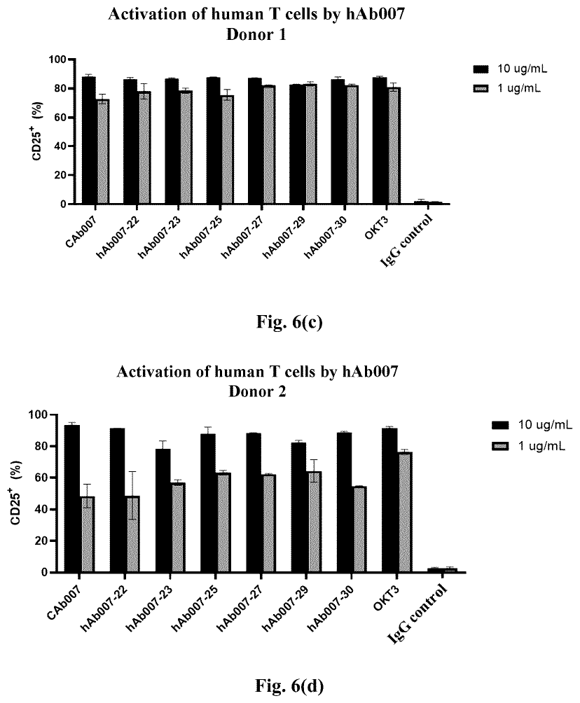

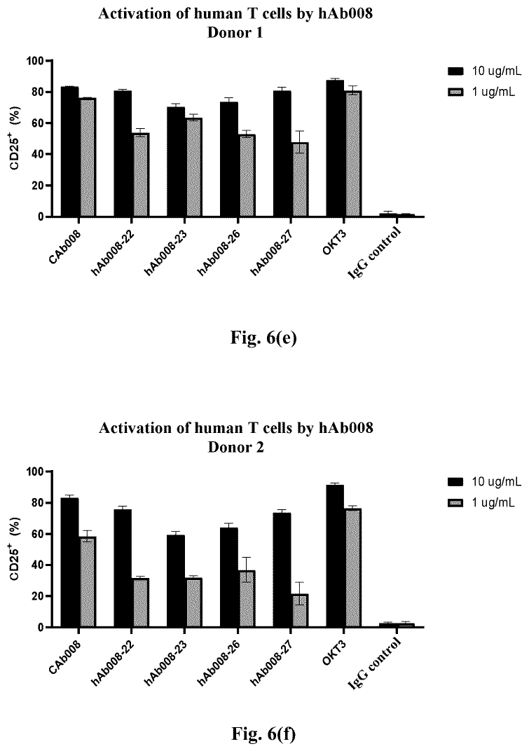

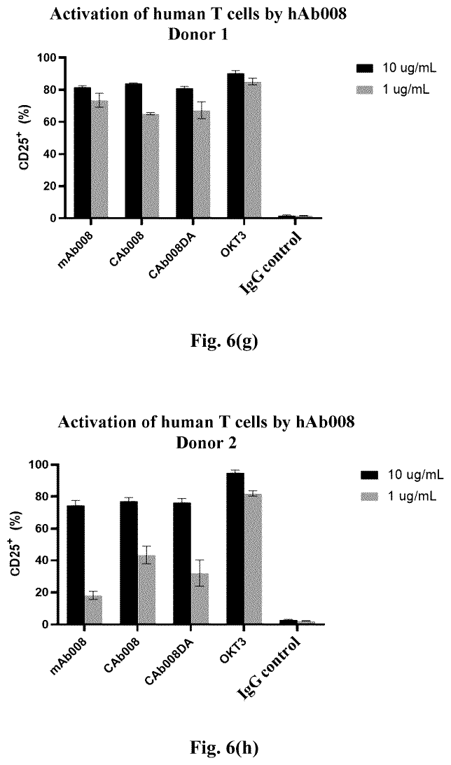

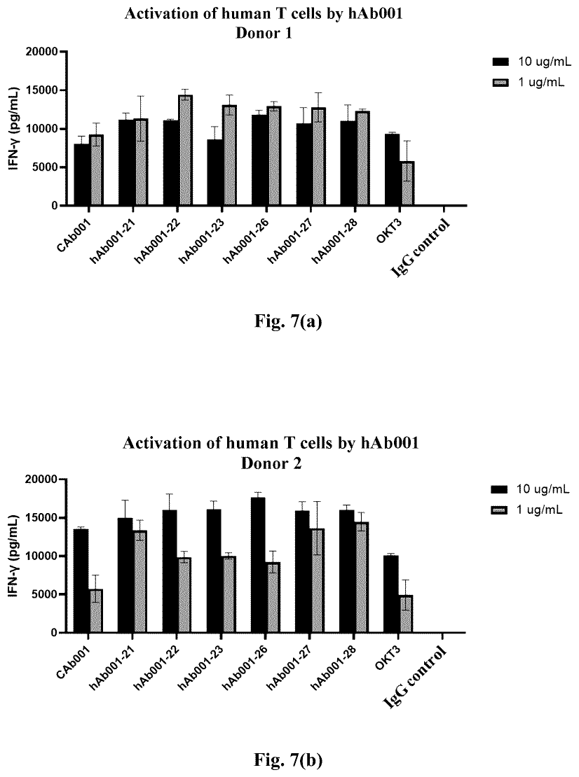

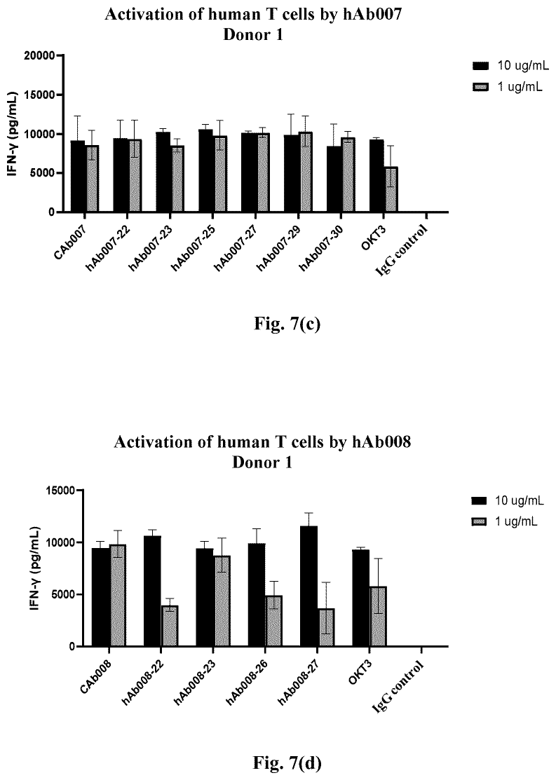

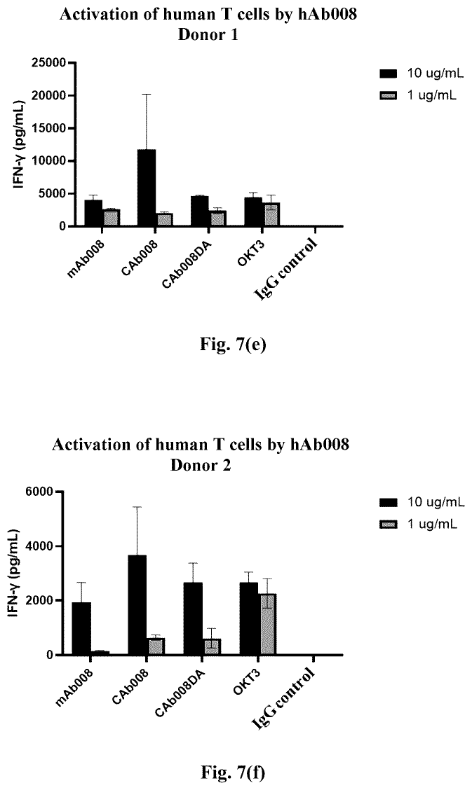

The results are shown in FIGS. 6 and 7. Both the humanized variants and the hotspot mutated variants of the CD3 antibodies can significantly activate total human T cells, which is specifically manifested by the up-regulated expression of CD25 and the release of IFN-γ.

### Example 7. Assay of Affinity of Antibodies for Human CD3e/g Antigen

The equilibrium dissociation constant of binding of CD3 antibodies to human CD3e/g antigen (purchased from ACRO, catalog number CDG-H52W5) was determined by kinetic binding method using Biacore 8K (GE) system. Through the capture method, the carboxylated dextran surface of CM5 chip was first activated with NHS (N-hydroxysuccinimide) and EDC (1-(3-dimethylaminopropyl)-3-ethylcarbodiimide hydrochloride), and then added with an anti-human IgG (Fc) antibody, the free amino group of which could form an amide bond with the activated carboxyl group, thereby being fixed on the chip surface. The anti-human IgG (Fc) antibody could bind to the Fc end of CD3 antibodies to capture the antibodies, and then a series of concentrations of antigen were added to obtain the binding and dissociation curves of antibody and antigen, and the corresponding kinetic constants were calculated by Biacore Evaluation software, as shown in the table below.

TABLE 7
K_{D} of CD3e/g antibodies and hCD3e/g as detected by Biacore

| Capture | Analyte | Chi² (RU²) | ka (1/Ms) | kd (1/s) | K_{D} (M) | Rmax (RU) |
|---|---|---|---|---|---|---|
| cAb001 | CD3 E&G | 7.13E+01 | 2.67E+05 | 6.16E−03 | 2.31E−08 | 97 |
| hAb001-17 | CD3 E&G | 3.85E+01 | 2.88E+05 | 4.52E−03 | 1.57E−08 | 53 |
| hAb001-18 | CD3 E&G | 1.63E+01 | 3.43E+05 | 1.21E−02 | 3.52E−08 | 76.5 |
| hAb001-19 | CD3 E&G | 2.91E+01 | 3.75E+05 | 3.54E−02 | 9.45E−08 | 169.7 |
| hAb001-20 | CD3 E&G | 2.31E+01 | 2.81E+05 | 3.03E−03 | 1.08E−08 | 44.2 |
| hAb001-21 | CD3 E&G | 1.30E+02 | 3.98E+05 | 6.69E−03 | 1.68E−08 | 126.7 |
| hAb001-22 | CD3 E&G | 3.77E+01 | 4.16E+05 | 8.48E−03 | 2.04E−08 | 92 |
| hAb001-23 | CD3 E&G | 9.45E+01 | 4.01E+05 | 6.96E−03 | 1.74E−08 | 117.6 |
| hAb001-24 | CD3 E&G | 8.32E+01 | 3.94E+05 | 6.55E−03 | 1.66E−08 | 103.8 |
| hAb001-25 | CD3 E&G | 1.69E+02 | 3.97E+05 | 8.96E−03 | 2.26E−08 | 248.3 |
| hAb001-26 | CD3 E&G | 1.60E+02 | 3.86E+05 | 6.73E−03 | 1.74E−08 | 165 |
| hAb001-27 | CD3 E&G | 1.81E+02 | 3.91E+05 | 8.23E−03 | 2.10E−08 | 206.4 |
| hAb001-28 | CD3 E&G | 9.61E+01 | 3.65E+05 | 7.45E−03 | 2.04E−08 | 136.5 |
| hAb001-29 | CD3 E&G | 7.84E+01 | 3.16E+05 | 7.22E−03 | 2.28E−08 | 103.4 |
| hAb001-30 | CD3 E&G | 6.91E+01 | 3.24E+05 | 8.35E−03 | 2.58E−08 | 108.2 |
| hAb001-31 | CD3 E&G | 1.11E+02 | 3.17E+05 | 6.24E−03 | 1.97E−08 | 107 |
| hAb001-32 | CD3 E&G | 5.81E+01 | 2.96E+05 | 4.97E−03 | 1.68E−08 | 70.8 |
| cAb007 | CD3 E&G | 6.48E+01 | 4.38E+05 | 5.32E−03 | 1.21E−08 | 71.8 |
| hAb007-17 | CD3 E&G | 2.64E+01 | 3.85E+05 | 2.25E−03 | 5.85E−09 | 38.4 |
| hAb007-18 | CD3 E&G | 2.71E+01 | 4.01E+05 | 2.41E−03 | 6.01E−09 | 38.2 |
| hAb007-19 | CD3 E&G | 8.47E+01 | 3.40E+05 | 3.17E−03 | 9.32E−09 | 66.5 |
| hAb007-20 | CD3 E&G | 9.87E+01 | 3.37E+05 | 3.75E−03 | 1.11E−08 | 73.4 |
| hAb007-21 | CD3 E&G | 3.35E+01 | 4.68E+05 | 3.77E−03 | 8.06E−09 | 45.4 |
| hAb007-22 | CD3 E&G | 1.01E+02 | 4.46E+05 | 5.94E−03 | 1.33E−08 | 90.6 |
| hAb007-23 | CD3 E&G | 3.96E+01 | 4.39E+05 | 2.90E−03 | 6.59E−09 | 45.7 |
| hAb007-24 | CD3 E&G | 3.39E+01 | 4.19E+05 | 2.58E−03 | 6.16E−09 | 44.1 |
| hAb007-25 | CD3 E&G | 4.80E+01 | 4.65E+05 | 4.30E−03 | 9.24E−09 | 57.1 |
| hAb007-26 | CD3 E&G | 8.65E+01 | 4.70E+05 | 5.65E−03 | 1.20E−08 | 84.4 |
| hAb007-27 | CD3 E&G | 7.23E+01 | 4.27E+05 | 4.27E−03 | 1.00E−08 | 70.4 |
| hAb007-28 | CD3 E&G | 8.88E+01 | 4.20E+05 | 4.94E−03 | 1.18E−08 | 82.2 |
| hAb007-29 | CD3 E&G | 5.48E+01 | 4.21E+05 | 4.15E−03 | 9.85E−09 | 59.7 |
| hAb007-30 | CD3 E&G | 2.53E+01 | 4.45E+05 | 3.16E−03 | 7.11E−09 | 36 |
| hAb007-31 | CD3 E&G | 1.43E+01 | 4.27E+05 | 2.25E−03 | 5.26E−09 | 29.1 |
| hAb007-32 | CD3 E&G | 3.16E+01 | 4.19E+05 | 3.20E−03 | 7.62E−09 | 42.6 |
| cAb008 | CD3 E&G | 1.59E+01 | 1.76E+05 | 3.72E−03 | 2.12E−08 | 182.3 |
| hAb008-16 | CD3 E&G | 4.60E−01 | 1.01E+05 | 1.89E−03 | 1.86E−08 | 14.5 |
| hAb008-17 | CD3 E&G | 6.81E+00 | 1.67E+05 | 1.26E−03 | 7.57E−09 | 33.3 |
| hAb008-18 | CD3 E&G | 2.62E+01 | 2.04E+05 | 6.91E−03 | 3.39E−08 | 206.7 |
| hAb008-19 | CD3 E&G | 4.60E+01 | 2.19E+05 | 5.73E−03 | 2.61E−08 | 160.2 |
| hAb008-20 | CD3 E&G | 2.60E+00 | 1.05E+05 | 1.52E−03 | 1.45E−08 | 29.3 |
| hAb008-21 | CD3 E&G | 2.04E+01 | 2.05E+05 | 3.82E−03 | 1.86E−08 | 63 |
| hAb008-22 | CD3 E&G | 3.01E+01 | 2.85E+05 | 5.88E−03 | 2.06E−08 | 190.1 |
| hAb008-23 | CD3 E&G | 3.80E+01 | 2.80E+05 | 4.87E−03 | 1.74E−08 | 167.1 |
| hAb008-24 | CD3 E&G | 3.63E+00 | 1.25E+05 | 2.69E−03 | 2.15E−08 | 35.9 |
| hAb008-25 | CD3 E&G | 3.74E+00 | 2.02E+05 | 7.41E−03 | 3.68E−08 | 52.4 |
| hAb008-26 | CD3 E&G | 3.99E+01 | 2.66E+05 | 4.82E−03 | 1.81E−08 | 224 |
| hAb008-27 | CD3 E&G | 2.27E+01 | 2.64E+05 | 4.75E−03 | 1.80E−08 | 139.2 |
| hAb008-28 | CD3 E&G | 1.47E+00 | 1.62E+05 | 4.41E−03 | 2.72E−08 | 24.5 |
| hAb008-29 | CD3 E&G | 1.38E+01 | 1.82E+05 | 3.78E−03 | 2.08E−08 | 63.5 |
| hAb008-30 | CD3 E&G | 3.93E+01 | 2.56E+05 | 5.22E−03 | 2.04E−08 | 161.1 |
| hAb008-31 | CD3 E&G | 3.76E+01 | 2.52E+05 | 3.81E−03 | 1.51E−08 | 136 |

It should be understood that after reading the above content of the present disclosure, those skilled in the art can make various changes or modifications to the present invention, and these equivalent versions also fall within the scope defined by the claims appended to this application.

The invention claimed is:

1. An antibody or antigen-binding fragment thereof that specifically binds to CD3, the heavy chain of which comprises CDR1, CDR2 and CDR3, wherein:
(a) the heavy chain CDR1 comprises a sequence that is at least 80% identical to SEQ ID NO: 1, SEQ ID NO: 2, SEQ ID NO: 3 or SEQ ID NO: 4;
(b) the heavy chain CDR2 comprises a sequence that is at least 80% identical to SEQ ID NO: 18, SEQ ID NO: 6, or SEQ ID NO: 22; and
(c) the heavy chain CDR3 comprises a sequence that is at least 80% identical to SEQ ID NO: 8, SEQ ID NO: 9, SEQ ID NO: 10 or SEQ ID NO: 11.

2. The antibody or antigen-binding fragment thereof according to claim 1, which comprises:
(a) a heavy chain CDR1 comprising a sequence selected from the group consisting of SEQ ID NO: 1, SEQ ID NO: 2, SEQ ID NO: 3, SEQ ID NO: 4, or a sequence having one amino acid addition, substitution and/or deletion compared to any of SEQ ID NOs: 1-4;
(b) a heavy chain CDR2 comprising a sequence selected from the group consisting of: WTYPGX₁X₂X₃IKYNEKFKG (SEQ ID NO: 5), SEQ ID NO: 6 or a sequence that differs from SEQ ID NO: 6 by no more than a total of 3 amino acid additions, substitutions and/or deletions, or YINPFX₄X₅YTKYNQKFKD (SEQ ID NO: 7); wherein X₁, X₂ and X₃ are each independently N or Q, X₄ is N or S, X₅ is S or D;
(c) a heavy chain CDR3 comprising a sequence selected from the group consisting of: SEQ ID NO: 8, SEQ ID NO: 9, SEQ ID NO: 10, SEQ ID NO: 11, or a sequence that differs from any of SEQ ID NOs: 8-11 by no more than a total of 2 amino acid additions, substitutions and/or deletions;
(d) a light chain CDR1 comprising a sequence selected from the group consisting of: KSSQSLLNX₆RTRKNYLA (SEQ ID NO: 12) or KSSQSLLDX₇DX₈KTYLN (SEQ ID NO: 13); wherein X₆ is N, Q or S, X₇ is S, A or G, X₈ is G or A;
(e) a light chain CDR2 comprising a sequence selected from the group consisting of: SEQ ID NO: 14 or SEQ ID NO: 15; and
(f) a light chain CDR3 comprising a sequence selected from the group consisting of: KQSX₉X₁₀LRT (SEQ ID NO: 16) or SEQ ID NO: 17; wherein X₉ is Y or F, and X₁₀ is T or I.

3. The antibody or antigen-binding fragment thereof according to claim 1, which comprises:
(1) a heavy chain CDR1 comprising or consisting of the following sequence: SEQ ID NO: 1; a heavy chain CDR2 comprising or consisting of the following sequence: WTYPGX₁X₂X₃IKYNEKFKG (SEQ ID NO: 5), wherein X₁, X₂ and X₃ are each independently N or Q; a heavy chain CDR3 comprising or consisting of the following sequence: SEQ ID NO: 8; a light chain CDR1 comprising or consisting of the following sequence: KSSQSLLNX₆RTRKNYLA (SEQ ID NO: 12), wherein X₆ is N or Q; a light chain CDR2 comprising or consisting of the following sequence: SEQ ID NO: 14; and a light chain CDR3 comprising or consisting of the following sequence: SEQ ID NO: 31; or
(2) a heavy chain CDR1 comprising or consisting of the following sequence: SEQ ID NO: 2; a heavy chain CDR2 comprising or consisting of the following sequence: SEQ ID NO: 6; a heavy chain CDR3 comprising or consisting of the following sequence: SEQ ID NO: 9; a light chain CDR1 comprising or consisting of the following sequence: SEQ ID NO: 26; a light chain CDR2 comprising or consisting of the following sequence: SEQ ID NO: 14; and a light chain CDR3 comprising or consisting of the following sequence: SEQ ID NO: 32; or
(3) a heavy chain CDR1 comprising or consisting of the following sequence: SEQ ID NO: 3; a heavy chain CDR2 comprising or consisting of the following sequence: SEQ ID NO: 22; a heavy chain CDR3 comprising or consisting of the following sequence: SEQ ID NO: 10; a light chain CDR1 comprising or consisting of the following sequence: KSSQSLLDX₇DX₈KTYLN (SEQ ID NO: 13), wherein X₇ is S, and X₈ is G or A; a light chain CDR2 comprising or consisting of the following sequence: SEQ ID NO: 15; and a light chain CDR3 comprising or consisting of the following sequence: SEQ ID NO: 17; or
(4) a heavy chain CDR1 comprising or consisting of the following sequence: SEQ ID NO: 4; a heavy chain CDR2 comprising or consisting of the following sequence: SEQ ID NO: 23; a heavy chain CDR3 comprising or consisting of the following sequence: SEQ ID NO: 11; a light chain CDR1 comprising or consisting of the following sequence: KSSQSLLDX₇DX₈KTYLN (SEQ ID NO: 13), wherein X₇ is G or A, and X₈ is G; a light chain CDR2 comprising or consisting of the following sequence: SEQ ID NO: 15; and a light chain CDR3 comprising or consisting of the following sequence: SEQ ID NO: 17.

4. The antibody or antigen-binding fragment thereof according to claim 2, characterized in that:
the heavy chain CDR2 comprises a sequence selected from the group consisting of SEQ ID NO: 18, SEQ ID NO: 19, SEQ ID NO: 20, SEQ ID NO: 21, SEQ ID NO: 6, SEQ ID NO: 22 or SEQ ID NO: 23; and/or
the light chain CDR1 comprises a sequence selected from the group consisting of SEQ ID NO: 24, SEQ ID NO: 25, SEQ ID NO: 26, SEQ ID NO: 27, SEQ ID NO: 28, SEQ ID NO: 29 or SEQ ID NO: 30; and/or
the light chain CDR3 comprises a sequence selected from the group consisting of SEQ ID NO: 31, SEQ ID NO: 32 or SEQ ID NO: 17.

5. The antibody or antigen-binding fragment thereof according to claim 1, which comprises CDRs as set forth in any one of the following (1) to (12):
(1) a heavy chain CDR1 comprising or consisting of the following sequence: SEQ ID NO: 1; a heavy chain CDR2 comprising or consisting of the following sequence: SEQ ID NO: 18; a heavy chain CDR3 comprising or consisting of the following sequence: SEQ ID NO: 8; a light chain CDR1 comprising or consisting of the following sequence: SEQ ID NO: 24; a light chain CDR2 comprising or consisting of the following sequence: SEQ ID NO: 14; and a light chain CDR3 comprising or consisting of the following sequence: SEQ ID NO: 31; or
(2) a heavy chain CDR1 comprising or consisting of the following sequence: SEQ ID NO: 1; a heavy chain CDR2 comprising or consisting of the following sequence: SEQ ID NO: 18; a heavy chain CDR3 comprising or consisting of the following sequence: SEQ ID NO: 8; a light chain CDR1 comprising or consisting of the following sequence: SEQ ID NO: 25; a light chain CDR2 comprising or consisting of the following sequence: SEQ ID NO: 14; and a light chain CDR3 comprising or consisting of the following sequence: SEQ ID NO: 31; or
(3) a heavy chain CDR1 comprising or consisting of the following sequence: SEQ ID NO: 1; a heavy chain CDR2 comprising or consisting of the following sequence: SEQ ID NO: 19; a heavy chain CDR3 comprising or consisting of the following sequence: SEQ ID NO: 8; a light chain CDR1 comprising or consisting of the following sequence: SEQ ID NO: 24; a light chain CDR2 comprising or consisting of the following sequence: SEQ ID NO: 14; and a light chain CDR3 comprising or consisting of the following sequence: SEQ ID NO: 31; or
(4) a heavy chain CDR1 comprising or consisting of the following sequence: SEQ ID NO: 1; a heavy chain CDR2 comprising or consisting of the following sequence: SEQ ID NO: 19; a heavy chain CDR3 comprising or consisting of the following sequence: SEQ ID NO: 8; a light chain CDR1 comprising or consisting of the following sequence: SEQ ID NO: 25; a light chain CDR2 comprising or consisting of the following sequence: SEQ ID NO: 14; and a light chain CDR3 comprising or consisting of the following sequence: SEQ ID NO: 31; or
(5) a heavy chain CDR1 comprising or consisting of the following sequence: SEQ ID NO: 1; a heavy chain CDR2 comprising or consisting of the following sequence: SEQ ID NO: 20; a heavy chain CDR3 comprising or consisting of the following sequence: SEQ ID NO: 8; a light chain CDR1 comprising or consisting of the following sequence: SEQ ID NO: 24; a light chain CDR2 comprising or consisting of the following sequence: SEQ ID NO: 14; and a light chain CDR3 comprising or consisting of the following sequence: SEQ ID NO: 31; or
(6) a heavy chain CDR1 comprising or consisting of the following sequence: SEQ ID NO: 1; a heavy chain CDR2 comprising or consisting of the following sequence: SEQ ID NO: 20; a heavy chain CDR3 comprising or consisting of the following sequence: SEQ ID NO: 8; a light chain CDR1 comprising or consisting of the following sequence: SEQ ID NO: 25; a light chain CDR2 comprising or consisting of the following sequence: SEQ ID NO: 14; and a light chain CDR3 comprising or consisting of the following sequence: SEQ ID NO: 31; or
(7) a heavy chain CDR1 comprising or consisting of the following sequence: SEQ ID NO: 1; a heavy chain CDR2 comprising or consisting of the following sequence: SEQ ID NO: 21; a heavy chain CDR3 comprising or consisting of the following sequence: SEQ ID NO: 8; a light chain CDR1 comprising or consisting of the following sequence: SEQ ID NO: 24; a light chain CDR2 comprising or consisting of the following sequence: SEQ ID NO: 14; and a light chain CDR3 comprising or consisting of the following sequence: SEQ ID NO: 31; or
(8) a heavy chain CDR1 comprising or consisting of the following sequence: SEQ ID NO: 1; a heavy chain CDR2 comprising or consisting of the following sequence: SEQ ID NO: 21; a heavy chain CDR3 comprising or consisting of the following sequence: SEQ ID NO: 8; a light chain CDR1 comprising or consisting of the following sequence: SEQ ID NO: 25; a light chain CDR2 comprising or consisting of the following sequence: SEQ ID NO: 14; and a light chain CDR3 comprising or consisting of the following sequence: SEQ ID NO: 31; or
(9) a heavy chain CDR1 comprising or consisting of the following sequence: SEQ ID NO: 3; a heavy chain CDR2 comprising or consisting of the following sequence: SEQ ID NO: 22; a heavy chain CDR3 comprising or consisting of the following sequence: SEQ ID NO: 10; a light chain CDR1 comprising or consisting of the following sequence: SEQ ID NO: 27; a light chain CDR2 comprising or consisting of the following sequence: SEQ ID NO: 15; and a light chain CDR3 comprising or consisting of the following sequence: SEQ ID NO: 17; or
(10) a heavy chain CDR1 comprising or consisting of the following sequence: SEQ ID NO: 3; a heavy chain CDR2 comprising or consisting of the following sequence: SEQ ID NO: 22; a heavy chain CDR3 comprising or consisting of the following sequence: SEQ ID NO: 10; a light chain CDR1 comprising or consisting of the following sequence: SEQ ID NO: 28; a light chain CDR2 comprising or consisting of the following sequence: SEQ ID NO: 15; and a light chain CDR3 comprising or consisting of the following sequence: SEQ ID NO: 17; or
(11) a heavy chain CDR1 comprising or consisting of the following sequence: SEQ ID NO: 4; a heavy chain CDR2 comprising or consisting of the following sequence: SEQ ID NO: 23; a heavy chain CDR3 comprising or consisting of the following sequence: SEQ ID NO: 11; a light chain CDR1 comprising or consisting of the following sequence: SEQ ID NO: 30; a light chain CDR2 comprising or consisting of the following sequence: SEQ ID NO: 15; and a light chain CDR3 comprising or consisting of the following sequence: SEQ ID NO: 17; or
(12) a heavy chain CDR1 comprising or consisting of the following sequence: SEQ ID NO: 4; a heavy chain CDR2 comprising or consisting of the following sequence: SEQ ID NO: 23; a heavy chain CDR3 comprising or consisting of the following sequence: SEQ ID NO: 11; a light chain CDR1 comprising or consisting of the following sequence: SEQ ID NO: 29; a light chain CDR2 comprising or consisting of the following sequence: SEQ ID NO: 15; and a light chain CDR3 comprising or consisting of the following sequence: SEQ ID NO: 17.

6. The antibody or antigen-binding fragment thereof according to claim 1, characterized in that the antibody or antigen-binding fragment thereof comprises a heavy chain variable region comprising an amino acid sequence selected from SEQ ID NOs: 33-57 or an amino acid sequence that is at least 85% identical to the foregoing sequences.

7. The antibody or antigen-binding fragment thereof according to claim 1, characterized in that the antibody or antigen-binding fragment thereof comprises a light chain variable region comprising an amino acid sequence selected from SEQ ID NOs: 58-82 or an amino acid sequence that is at least 85% identical to the foregoing sequences.

8. The antibody or antigen-binding fragment thereof according to claim 1, characterized in that the antibody or antigen-binding fragment thereof comprises the following set of heavy chain variable regions and light chain variable regions:
(1) a heavy chain variable region of an amino acid sequence selected from SEQ ID NO: 33, SEQ ID NO: 34, SEQ ID NO: 35 or SEQ ID NO: 36; and a light chain variable region selected from SEQ ID NO: 58, SEQ ID NO: 59, SEQ ID NO: 60 or SEQ ID NO: 61; or
(2) a heavy chain variable region of an amino acid sequence selected from SEQ ID NO: 37, SEQ ID NO: 38, SEQ ID NO: 39 or SEQ ID NO: 40; and a light chain variable region of an amino acid sequence selected from SEQ ID NO: 62, SEQ ID NO: 63, SEQ ID NO: 64 or SEQ ID NO: 65; or
(3) a heavy chain variable region of an amino acid sequence selected from SEQ ID NO: 41, SEQ ID NO: 42, SEQ ID NO: 43 or SEQ ID NO: 44; and a light chain variable region of an amino acid sequence selected from SEQ ID NO: 66, SEQ ID NO: 67, SEQ ID NO: 68 or SEQ ID NO: 69; or
(4) a heavy chain variable region of an amino acid sequence selected from SEQ ID NO: 45, SEQ ID NO: 46, SEQ ID NO: 47 or SEQ ID NO: 48; and a light chain variable region of an amino acid sequence selected from SEQ ID NO: 70, SEQ ID NO: 71, SEQ ID NO: 72 or SEQ ID NO: 73; or
(5) a heavy chain variable region of an amino acid sequence selected from SEQ ID NO: 49, SEQ ID NO: 50, SEQ ID NO: 51, SEQ ID NO: 52 or SEQ ID NO: 53; and a light chain variable region of an amino acid sequence selected from SEQ ID NO: 74, SEQ ID NO: 75 or SEQ ID NO: 76; or
(6) a heavy chain variable region of an amino acid sequence selected from SEQ ID NO: 54, SEQ ID NO: 55, SEQ ID NO: 56 or SEQ ID NO: 57; and a light chain variable region of an amino acid sequence selected from SEQ ID NO: 77, SEQ ID NO: 78, SEQ ID NO: 79 or SEQ ID NO: 80.

9. The antibody or antigen-binding fragment thereof according to claim 8, characterized in that the antibody or antigen-binding fragment thereof comprises the following set of the heavy chain variable region and the light chain variable region of any one of the following (1) to (18):
(1) a heavy chain variable region having an amino acid sequence of SEQ ID NO: 38, and a light chain variable region having an amino acid sequence of SEQ ID NO: 62; or
(2) a heavy chain variable region having an amino acid sequence of SEQ ID NO: 38 and a light chain variable region having an amino acid sequence of SEQ ID NO: 63; or
(3) a heavy chain variable region having an amino acid sequence of SEQ ID NO: 38 and a light chain variable region having an amino acid sequence of SEQ ID NO: 64; or
(4) a heavy chain variable region having an amino acid sequence of SEQ ID NO: 39 and a light chain variable region having an amino acid sequence of SEQ ID NO: 63; or
(5) a heavy chain variable region having an amino acid sequence of SEQ ID NO: 39, and a light chain variable region having an amino acid sequence of SEQ ID NO: 64; or
(6) a heavy chain variable region having an amino acid sequence of SEQ ID NO: 39 and a light chain variable region having an amino acid sequence of SEQ ID NO: 65; or
(7) a heavy chain variable region having an amino acid sequence of SEQ ID NO: 46, and a light chain variable region having an amino acid sequence of SEQ ID NO: 71; or
(8) a heavy chain variable region having an amino acid sequence of SEQ ID NO: 46, and a light chain variable region having an amino acid sequence of SEQ ID NO: 72; or
(9) a heavy chain variable region having an amino acid sequence of SEQ ID NO: 47 and a light chain variable region having an amino acid sequence of SEQ ID NO: 70; or
(10) a heavy chain variable region having an amino acid sequence of SEQ ID NO: 47 and a light chain variable region having an amino acid sequence of SEQ ID NO: 72; or
(11) a heavy chain variable region having an amino acid sequence of SEQ ID NO: 48 and a light chain variable region having an amino acid sequence of SEQ ID NO: 70; or
(12) a heavy chain variable region having an amino acid sequence of SEQ ID NO: 48 and a light chain variable region having an amino acid sequence of SEQ ID NO: 71; or
(13) a heavy chain variable region having an amino acid sequence of SEQ ID NO: 50 and a light chain variable region having an amino acid sequence of SEQ ID NO: 76; or
(14) a heavy chain variable region having an amino acid sequence of SEQ ID NO: 55 and a light chain variable region having an amino acid sequence of SEQ ID NO: 79; or
(15) a heavy chain variable region having an amino acid sequence of SEQ ID NO: 55, and a light chain variable region having an amino acid sequence of SEQ ID NO: 80; or
(16) a heavy chain variable region having an amino acid sequence of SEQ ID NO: 56, and a light chain variable region having an amino acid sequence of SEQ ID NO: 79; or
(17) a heavy chain variable region having an amino acid sequence of SEQ ID NO: 56, and a light chain variable region having an amino acid sequence of SEQ ID NO: 80; or
(18) a heavy chain variable region having an amino acid sequence of SEQ ID NO: 57, and a light chain variable region having an amino acid sequence of SEQ ID NO: 79.

10. The antibody or antigen-binding fragment thereof according to claim 1, characterized in that the antibody or antigen-binding fragment thereof is a human antibody, a humanized antibody, a chimeric antibody, a monoclonal antibody, a polyclonal antibody, a recombinant antibody, a single-chain antibody, a diabody, a three-chain antibody, a four-chain antibody, a Fab fragment, a F(ab′)₂ fragment, a scFv fragment, a Fv fragment, a Fab′ fragment, or a domain antibody.

11. The antibody or antigen-binding fragment thereof according to claim 10, characterized in that the antibody or antigen-binding fragment thereof binds to human CD3 and/or cynomolgus monkey CD3.

12. The antibody or antigen-binding fragment thereof according to claim 11, which binds to CD3e/d and/or CD3e/g.

13. The antibody or antigen-binding fragment thereof according to claim 11, characterized in that the antibody or antigen-binding fragment thereof (i) significantly activates total T cells, upregulates CD25 expression and/or induces the release of IFN-γ; and/or (ii) binds to human CD3 with a K_{D} value of less than 4×10⁻⁸ M.

14. An antibody or an antigen-binding fragment thereof, which competes for or cross-blocks the binding to CD3 with the antibody or antigen-binding fragment according to claim 1.

15. An isolated nucleic acid encoding the antibody or antigen-binding fragment thereof according to claim 1.

16.-22. (canceled)

23. A method for suppressing an immune response, activating T cells, treating cancer or an autoimmune disease, comprising administering to a subject in need thereof a therapeutically effective amount of the antibody or antigen-binding fragment thereof according to claim 1.

24. The method according to claim 23, wherein the cancer is selected from the group comprising melanoma, renal cancer, prostate cancer, breast cancer, colorectal cancer, lung cancer, nasopharyngeal cancer, oral cancer, liver cancer, bone cancer, pancreatic cancer, skin cancer, head and neck cancer, uterine cancer, ovarian cancer, rectal cancer, stomach cancer, testicular cancer, fallopian tube cancer, endometrial cancer, cervical cancer, vaginal cancer, vulvar cancer, Hodgkin's lymphoma, non-Hodgkin's lymphoma, esophageal cancer, small intestine cancer, large intestine cancer, endocrine system cancer, thyroid cancer, parathyroid cancer, adrenal cancer, soft tissue sarcoma, urethral cancer, penile cancer, chronic or acute leukemia, bladder cancer, renal pelvis cancer, central nervous system tumor, glioma, spinal axis tumor, brain stem glioma, pituitary adenoma, Kaposi's sarcoma, epidermoid carcinoma, squamous cell carcinoma, and T-cell lymphoma.

25. The method according to claim 23, wherein the autoimmune disease is selected from the group comprising rheumatoid arthritis, multiple sclerosis, Sjögren's syndrome, insulin-dependent diabetes mellitus, autoimmune thyroiditis, reactive arthritis, ankylosing spondylitis, scleroderma, polymyositis, dermatomyositis, psoriasis, Wegener's granulomatosis, Crohn's disease, ulcerative colitis, lupus such as systemic lupus erythematosus, atherosclerosis, chronic obstructive pulmonary disease, cirrhosis, renal transplant fibrosis, renal transplant nephropathy, and pulmonary fibrosis.

26. A method, wherein the antibody or antigen-binding fragment thereof according to claim is administered sequentially or simultaneously with another therapeutic agent.


SEQUENCE LISTING (92 sequences)

SEQ ID NO: 1 - protein, 5 aa, Artificial sequence
  Synthetic polypeptide
NYYMH

SEQ ID NO: 2 - protein, 5 aa, Artificial sequence
  Synthetic polypeptide
DNYIH

SEQ ID NO: 3 - protein, 5 aa, Artificial sequence
  Synthetic polypeptide
DYTVN

SEQ ID NO: 4 - protein, 5 aa, Artificial sequence
  Synthetic polypeptide
DYTIH

SEQ ID NO: 5 - protein, 17 aa, Artificial sequence
  Synthetic polypeptide
  MISC_FEATURE (6)..(8): Xaa can be Asn or Gln
WTYPGXXXIKYNEKFKG

SEQ ID NO: 6 - protein, 17 aa, Artificial sequence
  Synthetic polypeptide
WIYPGSVNIKYNEKFKD

SEQ ID NO: 7 - protein, 17 aa, Artificial sequence
  Synthetic polypeptide
  MISC_FEATURE (6)..(6): Xaa can be Asn or Ser
  MISC_FEATURE (7)..(7): Xaa can be Ser or Asp
YINPFXXYTKYNQKFKD

SEQ ID NO: 8 - protein, 10 aa, Artificial sequence
  Synthetic polypeptide
DGYGYYFFDY

SEQ ID NO: 9 - protein, 9 aa, Artificial sequence
  Synthetic polypeptide
DISRYYFDY

SEQ ID NO: 10 - protein, 5 aa, Artificial sequence
  Synthetic polypeptide
SVSTY

SEQ ID NO: 11 - protein, 5 aa, Artificial sequence
  Synthetic polypeptide
SVSIY

SEQ ID NO: 12 - protein, 17 aa, Artificial sequence
  Synthetic polypeptide
  MISC_FEATURE (9)..(9): Xaa can be Asn, Gln or Ser
KSSQSLLNXRTRKNYLA

SEQ ID NO: 13 - protein, 16 aa, Artificial sequence
  Synthetic polypeptide
  MISC_FEATURE (9)..(9): Xaa can be Ser, Ala or Gly
  MISC_FEATURE (11)..(11): Xaa can be Gly or Ala
KSSQSLLDXDXKTYLN

SEQ ID NO: 14 - protein, 7 aa, Artificial sequence
  Synthetic polypeptide
WASTRES

SEQ ID NO: 15 - protein, 7 aa, Artificial sequence
  Synthetic polypeptide
LVSKLNS

SEQ ID NO: 16 - protein, 8 aa, Artificial sequence
  Synthetic polypeptide
  MISC_FEATURE (4)..(4): Xaa can be Tyr or Phe
  MISC_FEATURE (5)..(5): Xaa can be Thr or Ile
KQSXXLRT

SEQ ID NO: 17 - protein, 9 aa, Artificial sequence
  Synthetic polypeptide
WQGTHFPRT

SEQ ID NO: 18 - protein, 17 aa, Artificial sequence
  Synthetic polypeptide
WTYPGNNNIKYNEKFKG

SEQ ID NO: 19 - protein, 17 aa, Artificial sequence
  Synthetic polypeptide
WTYPGQNNIKYNEKFKG

SEQ ID NO: 20 - protein, 17 aa, Artificial sequence
  Synthetic polypeptide
WTYPGNQNIKYNEKFKG

SEQ ID NO: 21 - protein, 17 aa, Artificial sequence
  Synthetic polypeptide
WTYPGQNQIKYNEKFKG

SEQ ID NO: 22 - protein, 17 aa, Artificial sequence
  Synthetic polypeptide
YINPFNSYTKYNQKFKD

SEQ ID NO: 23 - protein, 17 aa, Artificial sequence
  Synthetic polypeptide
YINPFSDYTKYNQKFKD

SEQ ID NO: 24 - protein, 17 aa, Artificial sequence
  Synthetic polypeptide
KSSQSLLNNRTRKNYLA

SEQ ID NO: 25 - protein, 17 aa, Artificial sequence
  Synthetic polypeptide
KSSQSLLNQRTRKNYLA

SEQ ID NO: 26 - protein, 17 aa, Artificial sequence
  Synthetic polypeptide
KSSQSLLNSRTRKNYLA

SEQ ID NO: 27 - protein, 16 aa, Artificial sequence
  Synthetic polypeptide
KSSQSLLDSDGKTYLN

SEQ ID NO: 28 - protein, 16 aa, Artificial sequence
  Synthetic polypeptide
KSSQSLLDSDAKTYLN

SEQ ID NO: 29 - protein, 16 aa, Artificial sequence
  Synthetic polypeptide
KSSQSLLDGDGKTYLN

SEQ ID NO: 30 - protein, 16 aa, Artificial sequence
  Synthetic polypeptide
KSSQSLLDADGKTYLN

SEQ ID NO: 31 - protein, 8 aa, Artificial sequence
  Synthetic polypeptide
KQSYTLRT

SEQ ID NO: 32 - protein, 8 aa, Artificial sequence
  Synthetic polypeptide
KQSFILRT

SEQ ID NO: 33 - protein, 119 aa, Artificial sequence
  Synthetic polypeptide
QVQLVQSGAEVKKPGASVKVSCKASGYSFTNYYMHWVRQATGQGLEWMGWTYPGNNNIKYNEKFKGRVTMTRNTSISTAYMELSSLRSEDTAVYYCARDGYGYYFFDYWGQGTLVTVSS

SEQ ID NO: 34 - protein, 119 aa, Artificial sequence
  Synthetic polypeptide
QVQLVQSGAEVKKPGASVKVSCKASGYSFTNYYMHWVRQATGQGLEWMGWTYPGQNNIKYNEKFKGRVTMTRNTSSSTAYMELSSLRSEDTAVYYCARDGYGYYFFDYWGQGTLVTVSS

SEQ ID NO: 35 - protein, 119 aa, Artificial sequence
  Synthetic polypeptide
QVQLVQSGAEVKKPGASVKVSCKASGYSFTNYYMHWVRQATGQGLEWMGWTYPGNQNIKYNEKFKGKVTMTRNTSISTAYMELSSLRSEDSAVYYCARDGYGYYFFDYWGQGTIVTVSS

SEQ ID NO: 36 - protein, 119 aa, Artificial sequence
  Synthetic polypeptide
QVQLVQSGAEVKKPGASVKVSCKASGYSFTNYYMHWVRQATGQGLEWMGWTYPGQNQIKYNEKFKGKVTMTRNTSSSTAYMELSSLRSEDSAVYYCARDGYGYYFFDYWGQGILVTVSS

SEQ ID NO: 37 - protein, 119 aa, Artificial sequence
  Synthetic polypeptide
QVQLVQSGAEVKKPGASVKVSCKASGYSFTNYYMHWVRQAPGQRLEWMGWTYPGNNNIKYNEKFKGRVTITRDTSASTAYMELSSLRSEDTAVYYCARDGYGYYFFDYWGQGTLVTVSS

SEQ ID NO: 38 - protein, 119 aa, Artificial sequence
  Synthetic polypeptide
QVQLVQSGAEVKKPGASVKVSCKASGYSFTNYYMHWVRQAPGQRLEWIGWTYPGNNNIKYNEKFKGRVTITADTSASTAYMELSSLRSEDTAVYYCARDGYGYYFFDYWGQGTLVTVSS

SEQ ID NO: 39 - protein, 119 aa, Artificial sequence
  Synthetic polypeptide
QVQLVQSGAEVKKPGASVKVSCKASGYSFTNYYMHWVRQAPGQRLEWIGWTYPGNNNIKYNEKFKGRATITADTSASTAYMELSSLRSEDTAVYYCARDGYGYYFFDYWGQGTLVTVSS

SEQ ID NO: 40 - protein, 119 aa, Artificial sequence
  Synthetic polypeptide
QVQLVQSGAEVKKPGASVKVSCKASGYSFTNYYMHWVKQAPGQRLEWIGWTYPGNNNIKYNEKFKGRATITADTSASTAYMELSSLRSEDTAVYYCARDGYGYYFFDYWGQGTLVTVSS

SEQ ID NO: 41 - protein, 118 aa, Artificial sequence
  Synthetic polypeptide
QVQLVQSGAEVKKPGASVKVSCKASGYTFTDNYIHWVRQATGQGLEWMGWIYPGSVNIKYNEKFKDRVTMTRNTSISTAYMELSSLRSEDTAVYYCARDISRYYFDYWGQGTLVTVSS

SEQ ID NO: 42 - protein, 118 aa, Artificial sequence
  Synthetic polypeptide
QVQLVQSGAEVKKPGASVKVSCKASGSTFTDNYIHWVRQATGQGLEWMGWIYPGSVNIKYNEKFKDKVTLTRNTSISTAYMELSSLRSEDSAVYYCARDISRYYFDYWGQGTLVTVSS

SEQ ID NO: 43 - protein, 118 aa, Artificial sequence
  Synthetic polypeptide
QVQLVQSGAEVVKPGASVKVSCKASGYTFTDNYIHWVRQRTGQGLEWMGWIYPGSVNIKYNEKFKDRVTMTADTSISTAYMQLSSLSSEDTAVYYCARDISRYYFDYWGQGTLVTVSS

SEQ ID NO: 44 - protein, 118 aa, Artificial sequence
  Synthetic polypeptide
QVQLVQSGAELVKPGASVKVSCKASGSTFTDNYIHWVRQRTGQGLEWMGWIYPGSVNIKYNEKFKDRVTLTADTSITTAYMQLSSLSSEDTAIYYCARDISRYYFDYWGQGTLVTVSS

SEQ ID NO: 45 - protein, 118 aa, Artificial sequence
  Synthetic polypeptide
QVQLVQSGAEVKKPGASVKVSCKASGSTFTDNYIHWVRQAPGQRLEWMGWIYPGSVNIKYNEKFKDRVTITRDTSASTAYMELSSLRSEDTAVYYCARDISRYYFDYWGQGTTVTVSS

SEQ ID NO: 46 - protein, 118 aa, Artificial sequence
  Synthetic polypeptide
QVQLVQSGAEVKKPGASVKVSCKASGSTFTDNYIHWVRQAPGQRLEWIGWIYPGSVNIKYNEKFKDRVTITADTSASTAYMELSSLRSEDTAVYYCARDISRYYFDYWGQGTTVTVSS

SEQ ID NO: 47 - protein, 118 aa, Artificial sequence
  Synthetic polypeptide
QVQLVQSGAEVKKPGASVKVSCKASGSTFTDNYIHWVRQAPGQRLEWIGWIYPGSVNIKYNEKFKDRATITADTSASTAYMELSSLRSEDTAVYYCARDISRYYFDYWGQGTTVTVSS

SEQ ID NO: 48 - protein, 118 aa, Artificial sequence
  Synthetic polypeptide
QVQLVQSGAEVKKPGASVKVSCKASGSTFTDNYIHWVKQAPGQRLEWIGWIYPGSVNIKYNEKFKDRATITADTSATTAYMELSSLRSEDTAVYYCARDISRYYFDYWGQGTTVTVSS

SEQ ID NO: 49 - protein, 114 aa, Artificial sequence
  Synthetic polypeptide
QVQLVQSGAEVKKPGSSVKVSCKASGYTFTDYTVNWVRQAPGQGLEWMGYINPFNSYTKYNQKFKDRVTITADESTSTAYMELSSLRSEDTAVYYCANSVSTYWGQGTLVTVSS

SEQ ID NO: 50 - protein, 114 aa, Artificial sequence
  Synthetic polypeptide
QVQLVQSGAEVAKPGSSVKVSCKASGYTFTDYTVNWVRQRPGQGLEWMGYINPFNSYTKYNQKFKDRVTITADESTSTAYMELSSLRSEDTAVYYCANSVSTYWGQGTLVTVSS

SEQ ID NO: 51 - protein, 114 aa, Artificial sequence
  Synthetic polypeptide
QVQLVQSGAEVAKPGSSVKVSCKASGYTFTDYTVNWVRQRPGQGLEWMGYINPFNSYTKYNQKFKDRVTITADESTSTAYMELSSLTSEDTAVYYCANSVSTYWGQGTLVTVSS

SEQ ID NO: 52 - protein, 114 aa, Artificial sequence
  Synthetic polypeptide
QVQLVQSGAEVAKPGSSVKVSCKTSGYTFTDYTVNWVRQRPGQGLEWMGYINPFNSYTKYNQKFKDRVTLTADESTSTAYMELSSLTSEDTAVYYCANSVSTYWGQGTLVTVSS

SEQ ID NO: 53 - protein, 114 aa, Artificial sequence
  Synthetic polypeptide
QVQLVQSGAEVAKPGSSVKVSCKTSGYTFTDYTVNWVRQRPGQGLEWMGYINPFNSYTKYNQKFKDRVTLTADKSTSTAYMQLSSLTSEDTAVYYCANSVSTYWGQGTLVTVSS

SEQ ID NO: 54 - protein, 114 aa, Artificial sequence
  Synthetic polypeptide
QVQLVQSGAEVKKPGASVKVSCKASGYTFTDYTVNWVRQAPGQRLEWMGYINPFNSYTKYNQKFKDRVTITRDTSASTAYMELSSLRSEDTAVYYCARSVSTYWGQGTTVTVSS

SEQ ID NO: 55 - protein, 114 aa, Artificial sequence
  Synthetic polypeptide
QVQLVQSGAEVKKPGASVKVSCKASGYTFTDYTVNWVRQAPGQRLEWIGYINPFNSYTKYNQKFKDRVTITADTSASTAYMELSSLRSEDTAVYYCANSVSTYWGQGTTVTVSS

SEQ ID NO: 56 - protein, 114 aa, Artificial sequence
  Synthetic polypeptide
QVQLVQSGAEVKKPGASVKVSCKASGYTFTDYTVNWVRQAPGQRLEWIGYINPFNSYTKYNQKFKDRATITADTSASTAYMELSSLRSEDTAVYFCANSVSTYWGQGTTVTVSS

SEQ ID NO: 57 - protein, 114 aa, Artificial sequence
  Synthetic polypeptide
QVQLVQSGAEVKKPGASVKVSCKASGYTFTDYTVNWVKQAPGQRLEWIGYINPFNSYTKYNQKFKDRATITADTSASTAYMELSSLRSEDTAVYFCANSVSTYWGQGTTVTVSS

SEQ ID NO: 58 - protein, 112 aa, Artificial sequence
  Synthetic polypeptide
DIQMTQSPSSLSASVGDRVTITCKSSQSLLNNRTRKNYLAWYQQKPGKAPKLLIYWASTRESGVPSRFSGSGSGTDFTLTISSLQPEDFATYYCKQSYTLRTFGGGTKVEIK

SEQ ID NO: 59 - protein, 112 aa, Artificial sequence
  Synthetic polypeptide
DIVMTQSPSSLSASVGDRVTITCKSSQSLLNQRTRKNYLAWYQQKPGKAPKLLIYWASTRESGVPSRFSGSGSGTDFTLTISSLQPEDFATYYCKQSYTLRTFGGGTKVEIK

SEQ ID NO: 60 - protein, 112 aa, Artificial sequence
  Synthetic polypeptide
DIQMTQSPSSLSASVGDRVTMTCKSSQSLLNNRTRKNYLAWYQQKPGKAPKLLIYWASTRESGVPSRFSGSGSGTDFTLTISSLQPEDFATYYCKQSYTLRTFGGGTKVEIK

SEQ ID NO: 61 - protein, 112 aa, Artificial sequence
  Synthetic polypeptide
DIVMTQSPSSLSASVGDRVTMTCKSSQSLLNQRTRKNYLAWYQQKPGKAPKLLIYWASTRESGVPSRFTGSGSGTDFTLTISSLQPEDFATYYCKQSYTLRTFGGGTKVEIK

SEQ ID NO: 62 - protein, 112 aa, Artificial sequence
  Synthetic polypeptide
DIVMTQSPDSLAVSLGERATINCKSSQSLLNNRTRKNYLAWYQQKPGQPPKLLIYWASTRESGVPDRFSGSGSGTDFTLTISSLQAEDVAVYYCKQSYTLRTFGGGTKLEIK

SEQ ID NO: 63 - protein, 112 aa, Artificial sequence
  Synthetic polypeptide
DIVMTQSPDSLAVSLGERATINCKSSQSLLNNRTRKNYLAWYQQKPGQPPKLLIYWASTRESGVPDRFTGSGSGTDFTLTISSLQAEDVAVYYCKQSYTLRTFGGGTKLEIK

SEQ ID NO: 64 - protein, 112 aa, Artificial sequence
  Synthetic polypeptide
DIVMTQSPDSLAVSLGERATMSCKSSQSLLNNRTRKNYLAWYQQKPGQPPKLLIYWASTRESGVPDRFSGSGSGTDFTLTISSLQAEDVAVYYCKQSYTLRTFGGGTKLEIK

SEQ ID NO: 65 - protein, 112 aa, Artificial sequence
  Synthetic polypeptide
DIVMTQSPDSLAVSLGERATMSCKSSQSLLNNRTRKNYLAWYQQKPGQPPKLLIYWASTRESGVPDRFTGSGSGTDFTLTISSLQAEDVAVYYCKQSYTLRTFGGGTKLEIK

SEQ ID NO: 66 - protein, 112 aa, Artificial sequence
  Synthetic polypeptide
DIQMTQSPSSLSASVGDRVTITCKSSQSLLNSRTRKNYLAWYQQKPGKAPKLLIYWASTRESGVPSRFSGSGSGTDFTLTISSLQPEDFATYYCKQSFILRTFGGGTKVEIK

SEQ ID NO: 67 - protein, 112 aa, Artificial sequence
  Synthetic polypeptide
DIQMTQSPSSLSASVGDRVTITCKSSQSLLNSRTRKNYLAWYQQKPGKSPKLLIYWASTRESGVPDRFTGSGSGTDFTLTISSLQPEDFATYYCKQSFILRTFGGGTKVAIK

SEQ ID NO: 68 - protein, 112 aa, Artificial sequence
  Synthetic polypeptide
DIQMSQSPSSLSASVGDRVTITCKSSQSLLNSRTRKNYLAWYQQKPGQSPKLLIYWASTRESGVPDRFTGSGSGTDFTLTISSLQAEDFATYYCKQSFILRTFGGGTKVAIK

SEQ ID NO: 69 - protein, 112 aa, Artificial sequence
  Synthetic polypeptide
DIVMSQSPSSLSASVGDRVTITCKSSQSLLNSRTRKNYLAWYQQKPGQSPKLLIYWASTRESGVPDRFTGSGSGTDFTLTISSVQPEDFATYYCKQSFILRTFGGGTKLAIK

SEQ ID NO: 70 - protein, 112 aa, Artificial sequence
  Synthetic polypeptide
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSFILRTFGGGTKLEIK

SEQ ID NO: 71 - protein, 112 aa, Artificial sequence
  Synthetic polypeptide
DIVMTQSPDSLAVSLGERATINCKSSQSLLNSRTRKNYLAWYQQKPGQPPKLLIYWASTRESGVPDRFTGSGSGTDFTLTISSLQAEDVAVYYCKQSFILRTFGGGTKLEIK

SEQ ID NO: 72 - protein, 112 aa, Artificial sequence
  Synthetic polypeptide
DIVMTQSPDSLAVSLGERATMNCKSSQSLLNSRTRKNYLAWYQQKPGQPPKLLIYWASTRESGVPDRFSGSGSGTDFTLTISSLQAEDVAVYYCKQSFILRTFGGGTKLEIK

SEQ ID NO: 73 - protein, 112 aa, Artificial sequence
  Synthetic polypeptide
DIVMTQSPDSLAVSLGERATMNCKSSQSLLNSRTRKNYLAWYQQKPGQPPKLLIYWASTRESGVPDRFTGSGSGTDFTLTISSLQAEDVAVYYCKQSFILRTFGGGTKLEIK

SEQ ID NO: 74 - protein, 112 aa, Artificial sequence
  Synthetic polypeptide
DVVMTQSPLSLPVTLGQPASISCKSSQSLLDSDGKTYLNWFQQRPGQSPRRLIYLVSKLNSGVPDRFSGSGSGTDFTLKISRVEAEDVGVYYCWQGTHFPRTFGGGTKVEIK

SEQ ID NO: 75 - protein, 112 aa, Artificial sequence
  Synthetic polypeptide
DVVMTQSPLSLPVTLGQPASISCKSSQSLLDSDGKTYLNWLQQRPGQSPRRLIYLVSKLNSGVPDRFSGSGSGTDFTLKISRVEAEDVGVYYCWQGTHFPRTFGGGTKVEIK

SEQ ID NO: 76 - protein, 112 aa, Artificial sequence
  Synthetic polypeptide
DVVMTQSPLSLPVTLGQPASISCKSSQSLLDSDGKTYLNWLQQRPGQSPRRLIQLVSKLNSGVPDRFSGSGSGTDFTLKISRVEAEDVGVYYCWQGTHFPRTFGGGTKVEIK

SEQ ID NO: 77 - protein, 112 aa, Artificial sequence
  Synthetic polypeptide
DVVMTQSPLSLPVTLGQPASISCKSSQSLLDSDGKTYLNWFQQRPGQSPRRLIYLVSKLNSGVPDRFSGSGSGTDFTLKISRVEAEDVGVYYCWQGTHFPRTFGGGTKLEIK

SEQ ID NO: 78 - protein, 112 aa, Artificial sequence
  Synthetic polypeptide
DVVMTQSPLSLPVTLGQPASISCKSSQSLLDSDGKTYLNWFQQRPGQSPRRLIQLVSKLNSGVPDRFSGSGSGTDFTLKISRVEAEDVGVFYCWQGTHFPRTFGGGTKLEIK

SEQ ID NO: 79 - protein, 112 aa, Artificial sequence
  Synthetic polypeptide
DVVMTQSPLSLPVTLGQPASISCKSSQSLLDSDGKTYLNWLQQRPGQSPRRLIQLVSKLNSGVPDRFSGSGSGTDFTLKISRVEAEDVGVFYCWQGTHFPRTFGGGTKLEIK

SEQ ID NO: 80 - protein, 112 aa, Artificial sequence
  Synthetic polypeptide
DVVMTQSPLSLPVTLGQPASISCKSSQSLLDSDGKTYLNWLLQRPGQSPRRLIQLVSKLNSGVPDRFSGSGSGTDFTLKISRVEAEDVGVFYCWQGTHFPRTFGGGTKLEIK

SEQ ID NO: 81 - protein, 112 aa, Artificial sequence
  Synthetic polypeptide
DVVMTQTPLTLSVTIGQPASISCKSSQSLLDSDAKTYLNWLLQRPGQSPKRLIQLVSKLNSGVPDRFTGSGSGTDFTLKISRVEAEDLGIFYCWQGTHFPRTFGGGTKLEIK

SEQ ID NO: 82 - protein, 112 aa, Artificial sequence
  Synthetic polypeptide
DVVMTQTPLTLSVTIGQPASISCKSSQSLLDADGKTYLNWLLQRPGQSPKRLIQLVSKLNSGVPDRFTGSGSGTDFTLKISRVEAEDLGIYYCWQGTHFPRTFGGGTKLEIK

SEQ ID NO: 83 - protein, 389 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 84 - protein, 644 aa, Macaca fascicularis
DGNEEMGSITQTPYQVSISGTTVILTCSQHLGSEAQWQHNGKNKEDSGDRLFLPEFSEMEQSGYYVCYPRGSNPEDASHHLYLKARVCENCMEMDGSSGSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKQSFEENRKLNVYNQEDGSVLLTCHVKNTNITWFKEGKMIDILTAHKNKWNLGSNTKDPRGVYQCKGSKDKSKTLQVYYRMCQNGSSGSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 85 - protein, 114 aa, Artificial sequence
  Synthetic polypeptide
QVQLQQSGAELTRPGASVKMSCKASGYTFTDYTIHWVKQRPGQGLEWIGYINPFSDYTKYNQKFKDKATLTADKSSSTAYIQLNSLTSEDSAVYYCANSVSIYWGQGTTLTVSS

SEQ ID NO: 86 - protein, 112 aa, Artificial sequence
  Synthetic polypeptide
DVVMTQTPLTLSVTIGQPASISCKSSQSLLDGDGKTYLNWLLQRPGQSPKRLIQLVSKLNSGVPDRFTGSGSGTDFTLKISRVEAEDLGIYYCWQGTHFPRTFGGGTKLEIK

SEQ ID NO: 87 - protein, 119 aa, Artificial sequence
  Synthetic polypeptide
QVQLQQSGPELVKPGASVKISCKASGYSFTNYYMHWVKQRPGQGLEWIGWTYPGNNNIKYNEKFKGKATLTADTSSSTAYMQLSSLTSEDSAVYYCARDGYGYYFFDYWGQGIILTVSS

SEQ ID NO: 88 - protein, 112 aa, Artificial sequence
  Synthetic polypeptide
DIVMSQSPSSLAVSAGEKVTMSCKSSQSLLNNRTRKNYLAWYQQKPGQSPKLLIYWASTRESGVPDRFTGSGSGTDFTLTISSVQAEDLAIYYCKQSYTLRTFGGGTKLAIK

SEQ ID NO: 89 - protein, 114 aa, Artificial sequence
  Synthetic polypeptide
QVQLQQSGAELARPGASVKMSCKTSGYTFTDYTVNWVKQRPGQGLEWIGYINPFNSYTKYNQKFKDKATLTADKSSSTAYMQLSSLTSEDSAVYFCANSVSTYWGQGTTLTVSS

SEQ ID NO: 90 - protein, 112 aa, Artificial sequence
  Synthetic polypeptide
DVVMTQTPLTLSVTIGQPASISCKSSQSLLDSDGKTYLNWLLQRPGQSPKRLIQLVSKLNSGVPDRFTGSGSGTDFTLKISRVEAEDLGIFYCWQGTHFPRTFGGGTKLEIK

SEQ ID NO: 91 - protein, 118 aa, Artificial sequence
  Synthetic polypeptide
QVQLQQSGPELVKPGASVKISCKASGSTFTDNYIHWVKQRPGQGLEWIGWIYPGSVNIKYNEKFKDKATLTADTSSTTAYMQLSSLSSEDSAIYYCARDISRYYFDYWGQGTTLTVSS

SEQ ID NO: 92 - protein, 112 aa, Artificial sequence
  Synthetic polypeptide
DIVMSQSPSSLAVSAGERVTMNCKSSQSLLNSRTRKNYLAWYQQKPGQSPKLLIYWASTRESGVPDRFTGSGSGTDFTLTISSVQAEDLAVYYCKQSFILRTFGGGTKLAIK